SEQUENCE LISTING (27 sequences)

SEQ ID NO: 1 - DNA, 21 bp, Escherichia coli
  source 1..21: mol_type = genomic DNA
aattgtgagcggataacaatt

SEQ ID NO: 2 - DNA, 21 bp, Escherichia coli
  source 1..21: mol_type = genomic DNA
aaatgtgagcgagtaacaacc

SEQ ID NO: 3 - DNA, 21 bp, Escherichia coli
  source 1..21: mol_type = genomic DNA
ggcagtgagcgcaacgcaatt

SEQ ID NO: 4 - DNA, 203 bp, Escherichia coli
  source 1..203: mol_type = genomic DNA
ataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttt

SEQ ID NO: 5 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDKDATAVEAACANVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLPPNTQTASPQVLADSLMQLARQISRLESGQ

SEQ ID NO: 6 - DNA, 3840 bp, synthetic construct
  misc_feature 1..3840: note = Recombinant plasmid
  source 1..3840: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgctagcagttaaccggagtactggtcgacctccgaagttgggggggagcaaaagcaggggaaaacaaaagcaacaaaaatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagcgtgaccacatggtccttcatgagtatgtaaatgctgctgggattacacatggcatggatgagctctacaaataacattaggatttcagaatcatgagaaaaacacccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 7 - DNA, 4043 bp, synthetic construct
  misc_feature 1..4043: note = Recombinant plasmid
  source 1..4043: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttctagcagttaaccggagtactggtcgacctccgaagttgggggggagcaaaagcaggggaaaacaaaagcaacaaaaatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccatggccaacacttgtcactactctgacctatggtgttcaatgcttttcccgttatccggatcacatgaaaaggcatgactttttcaagagtgccatgcccgaaggttatgtacaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaactcgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagcgtgaccacatggtccttcatgagtatgtaaatgctgctgggattacacatggcatggatgagctctacaaataacattaggatttcagaatcatgagaaaaacacccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 8 - DNA, 4795 bp, synthetic construct
  misc_feature 1..4795: note = Recombinant plasmid
  source 1..4795: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgctagcagttaaccggagtactggtcgacctccgaagttgggggggagcaaaagcaggggaaaatgattgcaatcataataatcgcggtagtggcctctaccagcaaatcagacaagatctgcattgggtatcatgccaacaactcgacaacacaagtggacacaatattagagaagaatgtgacagtgacgcactctgtagagctcctagaaagtcagaaggaggagagattctgcagagtgttgaataaaacacctctggatctaaagggttgcaccattgaaggatggattcttggaaacccccaatgtgacatcttacttggtgaccaaagttggtcatacatagtagagaggcctggagcccaaaatgggatatgttacccaggggtgctgaacgaagtggaagaactgaaagcattcattgggtccggagagaaagtacagagatttgaaatgtttcccaagagcacgtggaccggagtggacactaacagtggagttacgagagcttgcccctatactaccagtggatcatccttttacaggaatcttttgtggataataaaaacaaggtctgctgcatacccagtaattaagggaacatacaataatactggctcccagccaatcctatatttctggggtgtgcatcatcctccaaataccgatgagcaaaataccttatatggctctggtgacaggtatgttagaatgggaactgaaagcatgaattttgccaagagtcctgaaatagcagccaggccagctgtgaatgggcaaagaggaagaattgattattattggtctgtactgaaaccaggagaaaccttaaatgtagaatccaatggaaatttaatagctccttggtatgcttacaagttcacaagttccaacaacaaaggagctatcttcaaatcaaacctcccaattgagaattgtgatgctgtatgtcaaactgttgctggagcactaaagacaaacaaaactttccaaaatgttagtccactctggattggagaatgtcccaaatatgttaagagtgagagcctaagactggcaactggtctgaggaatgtcccacaggcagaaacaagaggattgtttggagccatagctgggtttatagaaggagggtggacaggtatgatagacggatggtacgggtaccatcatgagaactcacaggggtcgggttatgcagcagataaagaaagtacccagaaagcaattgacgggatcaccaataaagtaaattccatcattgacaagatgaacacacagtttgaagcagtagagcatgagttctcaaatctcgaaaggagaatagacaatttaaacaaaagaatggaagatggatttttggatgtgtggacgtacaatgctgaacttttagttctactggaaaatgaaaggaccctggatctgcacgatgccaatgtgaagaacctatacgagaaggtgaaatcacaattgagagataatgcaaaggatttgggtaatgggtgttttgaattttggcacaaatgcgacgatgaatgcatcaactcagttaagaatggcacatacgattacccaaagtaccaagacgagagcaaacttaacagacaggagatagactcagtgaagctggaaaatctgggcgtatatcaaattcttgctatttatagtacggtatcgagcagtctagttttggtggggctgatcattgccatgggtctttggatgtgctcaaatggctcaatgcaatgcaggatatgtatataattagaaaaaaacacccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 9 - DNA, 4998 bp, synthetic construct
  misc_feature 1..4998: note = Recombinant plasmid
  source 1..4998: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttctagcagttaaccggagtactggtcgacctccgaagttgggggggagcaaaagcaggggaaaatgattgcaatcataataatcgcggtagtggcctctaccagcaaatcagacaagatctgcattgggtatcatgccaacaactcgacaacacaagtggacacaatattagagaagaatgtgacagtgacgcactctgtagagctcctagaaagtcagaaggaggagagattctgcagagtgttgaataaaacacctctggatctaaagggttgcaccattgaaggatggattcttggaaacccccaatgtgacatcttacttggtgaccaaagttggtcatacatagtagagaggcctggagcccaaaatgggatatgttacccaggggtgctgaacgaagtggaagaactgaaagcattcattgggtccggagagaaagtacagagatttgaaatgtttcccaagagcacgtggaccggagtggacactaacagtggagttacgagagcttgcccctatactaccagtggatcatccttttacaggaatcttttgtggataataaaaacaaggtctgctgcatacccagtaattaagggaacatacaataatactggctcccagccaatcctatatttctggggtgtgcatcatcctccaaataccgatgagcaaaataccttatatggctctggtgacaggtatgttagaatgggaactgaaagcatgaattttgccaagagtcctgaaatagcagccaggccagctgtgaatgggcaaagaggaagaattgattattattggtctgtactgaaaccaggagaaaccttaaatgtagaatccaatggaaatttaatagctccttggtatgcttacaagttcacaagttccaacaacaaaggagctatcttcaaatcaaacctcccaattgagaattgtgatgctgtatgtcaaactgttgctggagcactaaagacaaacaaaactttccaaaatgttagtccactctggattggagaatgtcccaaatatgttaagagtgagagcctaagactggcaactggtctgaggaatgtcccacaggcagaaacaagaggattgtttggagccatagctgggtttatagaaggagggtggacaggtatgatagacggatggtacgggtaccatcatgagaactcacaggggtcgggttatgcagcagataaagaaagtacccagaaagcaattgacgggatcaccaataaagtaaattccatcattgacaagatgaacacacagtttgaagcagtagagcatgagttctcaaatctcgaaaggagaatagacaatttaaacaaaagaatggaagatggatttttggatgtgtggacgtacaatgctgaacttttagttctactggaaaatgaaaggaccctggatctgcacgatgccaatgtgaagaacctatacgagaaggtgaaatcacaattgagagataatgcaaaggatttgggtaatgggtgttttgaattttggcacaaatgcgacgatgaatgcatcaactcagttaagaatggcacatacgattacccaaagtaccaagacgagagcaaacttaacagacaggagatagactcagtgaagctggaaaatctgggcgtatatcaaattcttgctatttatagtacggtatcgagcagtctagttttggtggggctgatcattgccatgggtctttggatgtgctcaaatggctcaatgcaatgcaggatatgtatataattagaaaaaaacacccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 10 - DNA, 4774 bp, synthetic construct
  misc_feature 1..4774: note = Recombinant plasmid
  source 1..4774: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgctagcagttaaccggagtactggtcgacctccgaagttgggggggagcaaaagcaggggaaaatgattgcaatcataataatcgcggtagtggcctctaccagcaaatcagacaagatctgcattgggtatcatgccaacaactcgacaacacaagtggacacaatattagagaagaatgtgacagtgacgcactctgtagagctcctagaaagtcagaaggaggagagattctgcagagtgttgaataaaacacctctggatctaaagggttgcaccattgaaggatggattcttggaaacccccaatgtgacatcttacttggtgaccaaagttggtcatacatagtagagaggcctggagcccaaaatgggatatgttacccaggggtgctgaacgaagtggaagaactgaaagcattcattgggtccggagagaaagtacagagatttgaaatgtttcccaagagcacgtggaccggagtggacactaacagtggagttacgagagcttgcccctatactaccagtggatcatccttttacaggaatcttttgtggataataaaaacaaggtctgctgcatacccagtaattaagggaacatacaataatactggctcccagccaatcctatatttctggggtgtgcatcatcctccaaataccgatgagcaaaataccttatatggctctggtgacaggtatgttagaatgggaactgaaagcatgaattttgccaagagtcctgaaatagcagccaggccagctgtgaatgggcaaagaggaagaattgattattattggtctgtactgaaaccaggagaaaccttaaatgtagaatccaatggaaatttaatagctccttggtatgcttacaagttcacaagttccaacaacaaaggagctatcttcaaatcaaacctcccaattgagaattgtgatgctgtatgtcaaactgttgctggagcactaaagacaaacaaaactttccaaaatgttagtccactctggattggagaatgtcccaaatatgttaagagtgagagcctaagactggcaactggtctgaggaatgtcccacaggcagaaacaagaggattgtttggagccatagctgggtttatagaaggagggtggacaggtatgatagacggatggtacgggtaccatcatgagaactcacaggggtcgggttatgcagcagataaagaaagtacccagaaagcaattgacgggatcaccaataaagtaaattccatcattgacaagatgaacacacagtttgaagcagtagagcatgagttctcaaatctcgaaaggagaatagacaatttaaacaaaagaatggaagatggatttttggatgtgtggacgtacaatgctgaacttttagttctactggaaaatgaaaggaccctggatctgcacgatgccaatgtgaagaacctatacgagaaggtgaaatcacaattgagagataatgcaaaggatttgggtaatgggtgttttgaattttggcacaaatgcgacgatgaatgcatcaactcagttaagaatggcacatacgattacccaaagtaccaagacgagagcaaacttaacagacaggagatagactcagtgaagctggaaaatctgggcgtatatcaaattcttgctatttatagtacggtatcgagcagtctagttttggtggggctgatcattgccatgggtctttggatgtgctcaaatggctcaatgcaatgcaggatatgtatataattagaaaaaaacacccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 11 - DNA, 4505 bp, synthetic construct
  misc_feature 1..4505: note = Recombinant plasmid
  source 1..4505: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgctagcagttaaccggagtactggtcgacctccgaagttgggggggagcaaaagcaggagtttaaaatgaatccaaatcaaaagataataaccattgggtcaatctgcatggcaattggaataataagtctggtgttacaaattggaaatataatctcaatatgggttagtcattcaattcagactggaagtcagagccaccctgaaacatgcaatcaaagtgtcattacctacgaaaacaatacttgggtaaatcaaacatacgtcaacataagtaataccaatttgattgcagaacagactgtagctccagtaacactagcaggcaattcctctctctgtcccatcagtggatgggctatatacagcaaggacaatggtataaggataggttctaagggagatgtatttgtcatcagagagccttttatttcatgctctcacttggaatgcaggactttctttctaactcaaggggccttgttgaatgacaagcattccaatggaaccgttaaagacagaagcccctatagaaccctaatgagctgtcctgttggtgaagctccctctccatacaattcaaggtttgagtctgttgcttggtcggcaagtgcttgccacgatggcattagttggttgacaattggtatttccggccctgataatggggcggtggctgtattgaaatacaatggcataataacagatactatcaagagttggagaaataacatattgagaacacaagagtctgaatgtgcctgcattaatggttcttgctttaccataatgactgatggaccaagtaatggccaggcctcatacaagattttcaagatagaaaagggaaaggtagtcaaatcagttgagttgaatgcccctaattaccactatgaggagtgttcctgttatcctgatgctagcgaggtgatgtgtgtatgcagagacaactggcatggttcaaatcgaccatgggtgtccttcgatcagaatctagagtatcaaataggatacatatgcagcggagtttttggagacaatccacgccccaatgatgggacaggcagttgtggtccagtgtcttctaatggggcatatggggtaaaagggttttcatttaaatacggcaacggtgtttggataggaagaactaaaagtactagctcaaggagcggatttgagatgatttgggatcccaatggatggacagagacggacaacagtttctctgtgaagcaagacattgtagcaataactgattggtcaggatatagcggaagttttgttcagcatccagagctgacaggactagactgcatgagaccttgcttctgggttgagctaatcaggggaagacccaaggagaatacaatctggaccagtgggagcagcatttccttttgtggagtaaatagcgacactgtgggttggtcttggccagacggtgctgagttgccattcaccattgacaagtagtttgttcaaaaaactccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 12 - DNA, 4708 bp, synthetic construct
  misc_feature 1..4708: note = Recombinant plasmid
  source 1..4708: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattaggcagtgagcgcaacgcaattgtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttctagcagttaaccggagtactggtcgacctccgaagttgggggggagcaaaagcaggagtttaaaatgaatccaaatcaaaagataataaccattgggtcaatctgcatggcaattggaataataagtctggtgttacaaattggaaatataatctcaatatgggttagtcattcaattcagactggaagtcagagccaccctgaaacatgcaatcaaagtgtcattacctacgaaaacaatacttgggtaaatcaaacatacgtcaacataagtaataccaatttgattgcagaacagactgtagctccagtaacactagcaggcaattcctctctctgtcccatcagtggatgggctatatacagcaaggacaatggtataaggataggttctaagggagatgtatttgtcatcagagagccttttatttcatgctctcacttggaatgcaggactttctttctaactcaaggggccttgttgaatgacaagcattccaatggaaccgttaaagacagaagcccctatagaaccctaatgagctgtcctgttggtgaagctccctctccatacaattcaaggtttgagtctgttgcttggtcggcaagtgcttgccacgatggcattagttggttgacaattggtatttccggccctgataatggggcggtggctgtattgaaatacaatggcataataacagatactatcaagagttggagaaataacatattgagaacacaagagtctgaatgtgcctgcattaatggttcttgctttaccataatgactgatggaccaagtaatggccaggcctcatacaagattttcaagatagaaaagggaaaggtagtcaaatcagttgagttgaatgcccctaattaccactatgaggagtgttcctgttatcctgatgctagcgaggtgatgtgtgtatgcagagacaactggcatggttcaaatcgaccatgggtgtccttcgatcagaatctagagtatcaaataggatacatatgcagcggagtttttggagacaatccacgccccaatgatgggacaggcagttgtggtccagtgtcttctaatggggcatatggggtaaaagggttttcatttaaatacggcaacggtgtttggataggaagaactaaaagtactagctcaaggagcggatttgagatgatttgggatcccaatggatggacagagacggacaacagtttctctgtgaagcaagacattgtagcaataactgattggtcaggatatagcggaagttttgttcagcatccagagctgacaggactagactgcatgagaccttgcttctgggttgagctaatcaggggaagacccaaggagaatacaatctggaccagtgggagcagcatttccttttgtggagtaaatagcgacactgtgggttggtcttggccagacggtgctgagttgccattcaccattgacaagtagtttgttcaaaaaactccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 13 - DNA, 4484 bp, synthetic construct
  misc_feature 1..4484: note = Recombinant plasmid
  source 1..4484: mol_type = other DNA
actctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaattgtgagcggataacaattagacccaagctgttaacgctagcagttaaccggagtactggtcgacctccgaagttgggggggagcaaaagcaggagtttaaaatgaatccaaatcaaaagataataaccattgggtcaatctgcatggcaattggaataataagtctggtgttacaaattggaaatataatctcaatatgggttagtcattcaattcagactggaagtcagagccaccctgaaacatgcaatcaaagtgtcattacctacgaaaacaatacttgggtaaatcaaacatacgtcaacataagtaataccaatttgattgcagaacagactgtagctccagtaacactagcaggcaattcctctctctgtcccatcagtggatgggctatatacagcaaggacaatggtataaggataggttctaagggagatgtatttgtcatcagagagccttttatttcatgctctcacttggaatgcaggactttctttctaactcaaggggccttgttgaatgacaagcattccaatggaaccgttaaagacagaagcccctatagaaccctaatgagctgtcctgttggtgaagctccctctccatacaattcaaggtttgagtctgttgcttggtcggcaagtgcttgccacgatggcattagttggttgacaattggtatttccggccctgataatggggcggtggctgtattgaaatacaatggcataataacagatactatcaagagttggagaaataacatattgagaacacaagagtctgaatgtgcctgcattaatggttcttgctttaccataatgactgatggaccaagtaatggccaggcctcatacaagattttcaagatagaaaagggaaaggtagtcaaatcagttgagttgaatgcccctaattaccactatgaggagtgttcctgttatcctgatgctagcgaggtgatgtgtgtatgcagagacaactggcatggttcaaatcgaccatgggtgtccttcgatcagaatctagagtatcaaataggatacatatgcagcggagtttttggagacaatccacgccccaatgatgggacaggcagttgtggtccagtgtcttctaatggggcatatggggtaaaagggttttcatttaaatacggcaacggtgtttggataggaagaactaaaagtactagctcaaggagcggatttgagatgatttgggatcccaatggatggacagagacggacaacagtttctctgtgaagcaagacattgtagcaataactgattggtcaggatatagcggaagttttgttcagcatccagagctgacaggactagactgcatgagaccttgcttctgggttgagctaatcaggggaagacccaaggagaatacaatctggaccagtgggagcagcatttccttttgtggagtaaatagcgacactgtgggttggtcttggccagacggtgctgagttgccattcaccattgacaagtagtttgttcaaaaaactccttgtttctactaataacccggcggcccaaaatgccgaaatgtgagcgagtaacaaccactcggagcgaaagatatacctcccccggggccgggaggtcgcgtcaccgaccacgccgccggcccaggcgacgcgcgacacggacacctgtccccaaaaacgccaccatcgcagccacacacggagcgcccggggccctctggtcaaccccaggacacacgcgggagcagcgccgggccggggacgccctcccggcggtgacctggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaa

SEQ ID NO: 14 - DNA, 31 bp, synthetic construct
  misc_feature 1..31: note = Primer
  source 1..31: mol_type = other DNA
gtttctactaataacccggcggcccaaaatg

SEQ ID NO: 15 - DNA, 41 bp, synthetic construct
  misc_feature 1..41: note = Primer
  source 1..41: mol_type = other DNA
cctgcttttgctcccccccaacttcggaggtcgaccagtac

SEQ ID NO: 16 - DNA, 49 bp, synthetic construct
  misc_feature 1..49: note = Primer
  source 1..49: mol_type = other DNA
ctggtcgacctccgaagttgggggggagcaaaagcaggagtttaaaatg

SEQ ID NO: 17 - DNA, 48 bp, synthetic construct
  misc_feature 1..48: note = Primer
  source 1..48: mol_type = other DNA
gcattttgggccgccgggttattagtagaaacaaggagttttttgaac

SEQ ID NO: 18 - DNA, 49 bp, synthetic construct
  misc_feature 1..49: note = Primer
  source 1..49: mol_type = other DNA
ggtcgacctccgaagttgggggggagcaaaagcaggggaaaacaaaagc

SEQ ID NO: 19 - DNA, 47 bp, synthetic construct
  misc_feature 1..47: note = Primer
  source 1..47: mol_type = other DNA
gcattttgggccgccgggttattagtagaaacaagggtgtttttctc

SEQ ID NO: 20 - DNA, 46 bp, synthetic construct
  misc_feature 1..46: note = Primer
  source 1..46: mol_type = other DNA
ctggtcgacctccgaagttgggggggagcaaaagcaggggaaaatg

SEQ ID NO: 21 - DNA, 58 bp, synthetic construct
  misc_feature 1..58: note = Primer
  source 1..58: mol_type = other DNA
gcattttgggccgccgggttattagtagaaacaagggtgtttttttctaattatatac

SEQ ID NO: 22 - DNA, 24 bp, synthetic construct
  misc_feature 1..24: note = Primer
  source 1..24: mol_type = other DNA
agcagttaaccggagtactggtcg

SEQ ID NO: 23 - DNA, 42 bp, synthetic construct
  misc_feature 1..42: note = Synthetic nucleic acid
  source 1..42: mol_type = other DNA
ggattcatcagtgctgagttgccattcaccattgacaagtag

SEQ ID NO: 24 - DNA, 48 bp, synthetic construct
  misc_feature 1..48: note = Synthetic nucleic acid
  source 1..48: mol_type = other DNA
gtggtgtgtgtgtgcagagacaatcggcatgcttcgaatcgaccatgg

SEQ ID NO: 25 - DNA, 32 bp, synthetic construct
  misc_feature 1..32: note = Synthetic nucleic acid
  source 1..32: mol_type = other DNA
gtgctgagttgccattcaccattgacaagtag

SEQ ID NO: 26 - DNA, 42 bp, synthetic construct
  misc_feature 1..42: note = Synthetic nucleic acid
  source 1..42: mol_type = other DNA
aacacacagtttgaagcagtagagcatgagtctgatgaatcc

SEQ ID NO: 27 - DNA, 42 bp, synthetic construct
  misc_feature 1..42: note = Synthetic nucleic acid
  source 1..42: mol_type = other DNA
ggggatctctcagagcatgagttctcaaatctcgaaaggaga

(19) United States
(12) Patent Application Publication
(10) Pub. No.: US 2025/0146006 A1
(43) Pub. Date: May 8, 2025

(54) RECOMBINANT NUCLEIC ACID MOLECULES AND PLASMIDS FOR INCREASING STABILITY OF GENES TOXIC TO E. COLI

(71) Applicants: Jan-Willem de Gier, Stockholm (SE); The U.S.A., as represented by the Secretary, Department of Health and Human Services, Silver Spring, MD (US)

(72) Inventors: Robert D. Daniels, Silver Spring, MD (US); Jan-Willem de Gier, Stockholm (SE); Tahir H. Malik, Silver Spring, MD (US)

(73) Assignee: The U.S.A., as represented by the Secretary, Department of Health and Human Services, Silver Spring, MD (US)

(21) Appl. No.: 18/867,911

(22) PCT Filed: May 26, 2023
(86) PCT No.: PCT/US2023/067544

§ 371 Date: Nov. 21, 2024

Related U.S. Application Data
(60) Provisional application No. 63/346,568, filed on May 27, 2022.

(51) Int. Cl. C12N 15/72

(52) U.S. Cl. CPC C12N 15/72

(57) ABSTRACT

Recombinant nucleic acid molecules engineered for efficient propagation of a heterologous DNA sequence (such as a heterologous viral gene) that is toxic in E. coli are described. Influenza virus hemagglutinin (HA) and neuraminidase (NA) genes were used as exemplary toxic heterologous DNA sequences. Plasmids that included a heterologous DNA positioned between bacterial regulatory elements (such as lac operator sequences and/or terminator sequences) exhibited decreased gene transcription in E. coli and increased stability of the heterologous DNA while also retaining the property of inducible expression in E. coli.

### CROSS REFERENCE TO RELATED APPLICATIONS

This application claims the benefit of U.S. Provisional Application No. 63/346,568, filed May 27, 2022, which is herein incorporated by reference in its entirety.

### FIELD

This disclosure concerns recombinant nucleic acid molecules and plasmids for cloning and expressing heterologous DNA sequences (such as genes) that are toxic to Escherichia coli.

### INCORPORATION OF ELECTRONIC SEQUENCE LISTING

The electronic sequence listing, submitted herewith as an XML file named 9531-107951-02.xml (69,915 bytes), created on May 15, 2023, is herein incorporated by reference in its entirety.

### BACKGROUND

Plasmid-based viral reverse genetic systems enable viral genomes to be rapidly modified in a directed manner, providing molecular details that were not previously possible. Reverse genetics systems for RNA viruses were initially developed in the 1980's and are now commonly used to investigate pathogenesis and viral replication processes. More recently, viral reverse genetic systems have been utilized to incorporate changes that attenuate a virus or induce a more robust immune response to manufacture “customized” component-based or virus-based vaccines. Despite these advances and applications, plasmid- based reverse genetics are still limited by the ability to generate the viral genome-containing plasmid, propagate it in bacteria, and ultimately produce infectious virus.

Reverse genetic systems for influenza A viruses (IAVs) have been instrumental for addressing key questions about the viral life cycle and for developing new influenza vaccine strategies. The first systems involved the transfection of twelve or sixteen plasmids into mammalian cells; eight human RNA polymerase I (PolI) promoter driven plasmids for transcribing the eight negative-sense viral RNA (vRNA) genome segments, and either four or eight cytomegalovirus (CMV) polymerase II (PolII) promoter driven plasmids for transcribing all of the viral mRNAs or only the mRNAs encoding the nucleoprotein (NP) and the three polymerase subunits. Another IAV reverse genetics system described by Hoffmann et al. (Proc Natl Acad Sci USA 97(11):6108-6113, 2000) uses bidirectional constructs for efficiently generating IAVs from eight plasmids. In this system, each plasmid contains one IAV gene segment flanked by a PolI and a PolII promoter resulting in the transcription of both vRNA and mRNA from all eight gene segments following co-transfection into 293T cells cultured together with MDCK cells.

Multiple studies have reported difficulties cloning several IAV gene segments (for example, PB2, PB1, and HA) into established reverse genetics plasmids, suggesting these influenza virus genes are toxic to E. coli. This challenge of cloning viral genes or cDNAs is not unique to influenza viruses; it has also been reported for genes from flaviviruses (e.g., dengue virus and Kunjin virus), CMV, Rous sarcoma virus and hepatitis B virus. However, mechanistic data explaining these observations is lacking and the studies that have investigated toxic or unstable viral genes generally conclude the toxicity is a result of viral gene expression in E. coli. Supporting this possibility, cryptic E. coli promoter-like sequences have been identified in the CMV promoter, which is a common feature in several viral reverse genetics plasmids and eukaryotic expression vectors. In addition, regions in the viral genomes themselves (e.g., the 5′ UTR of dengue and Kinjun viruses, the 5′ LTR of Rous sarcoma virus and the hepatitis B virus precore region) have been shown to facilitate transcription in E. coli.

For IAV reverse genetics, different approaches have been reported for increasing the stability of viral gene segments that appear toxic. These include the use of reverse genetics plasmids that contain low copy number E. coli origins of replication, recombination-deficient E. coli strains (e.g., HB101), and lower growth temperatures (30-32° C.) for the transformed bacteria. Although each of these approaches have advantages, none of them provide a universal solution for cloning potentially toxic gene targets that require amplification in E. coli for DNA isolation or protein production. Thus, a need exists for the development of reagents and methods that allow for cloning of heterologous DNA sequences (such as genes) that are toxic to E. coli.

### SUMMARY

The present disclosure describes recombinant nucleic acid molecules engineered for efficient propagation of a heterologous DNA sequence (such as a heterologous viral gene) that is toxic in E. coli. It is disclosed herein that exemplary toxic heterologous DNA sequences cloned into plasmids can be transcribed and translated in E. coli and that the toxicity of the heterologous DNA is mitigated by introducing regulatory elements that decrease gene transcription in E. coli.

Provided herein are recombinant nucleic acid molecules that include, in the 5′ to 3′ direction, a first lac operator sequence, a heterologous DNA sequence, and a second lac operator sequence. Also provided herein are recombinant nucleic acid molecules that include, in the 5′ to 3′ direction, a first lac operator sequence, a multiple cloning site for insertion of a heterologous DNA sequence, and a second lac operator sequences. In some aspects, the heterologous DNA sequence encodes a protein or transcript that is toxic to E. coli.

In some aspects, the recombinant nucleic acid molecule further includes a first promoter located 5′ of the first lac operator sequence or located 3′ of the second lac operator sequence. In some examples, the recombinant nucleic acid molecule further includes a first promoter located 5′ of the first lac operator sequence and a second promoter located 3′ of the second lac operator sequence. The first promoter and/or second promoter can be a bacterial promoter (such as, but not limited to, an E. coli RNA polymerase promoter, T7 promoter or T4 promoter) or a mammalian promoter (such as, but not limited to, an RNA polymerase I promoter, RNA polymerase II promoter or RNA polymerase III promoter). In specific examples, the recombinant nucleic acid molecule further includes a third lac operator sequence located 5′ of the first promoter or located 3′ of the second promoter.

Also provided herein are plasmids, such as expression plasmids or cloning plasmids, that include a recombinant nucleic acid molecule disclosed herein. In some aspects of the disclosed plasmids, the heterologous DNA sequence is a viral gene, such as a gene encoding an influenza virus hemagglutinin (HA) or neuraminidase (NA) protein.

Further provided herein are methods of propagating a plasmid in E. coli, wherein the plasmid includes a heterologous DNA sequence that is toxic to E. coli. In some aspects, the method includes transforming E. coli with a disclosed plasmid under conditions sufficient to allow replication of the plasmid, thereby propagating the plasmid in E. coli.

Kits that include a recombinant nucleic acid molecule or a plasmid disclosed herein are also provided. The kits can further include, for example, one or more restriction endonucleases, one or more ligases, buffer, culture media, one or more antibiotics, or a combination thereof. In some examples, the kits include E. coli cells, which in some examples are frozen, in a liquid culture, or in a solid culture. Components of a kit can be present in separate vials or containers.

Also provided are isolated cells that include a recombinant nucleic acid molecule disclosed herein. In one example, the cells are E. coli cells. In the isolated cells, the recombinant nucleic acid molecule is capable of forming a complex with an Escherichia coli Lac repressor protein or a variant thereof.

The foregoing and other features of this disclosure will become more apparent from the following detailed description of several aspects which proceeds with reference to the accompanying figures.

### BRIEF DESCRIPTION OF THE DRAWINGS

FIGS. 1A-1F: Construction of human and avian H1N1 NA plasmid libraries for influenza reverse genetics. (FIG. 1A) Schematic of the cloning strategy for creating the human and avian NA subtype 1 (N1) plasmid libraries. Each N1 gene segment was cloned into the pHW plasmid using a simplified version of the PCR-based Gibson assembly method. (FIG. 1B) Table displaying the number of human and avian N1 gene segments that were readily cloned into the pHW plasmid. The asterisk denotes that three avian N1 gene segments (from 1983, 1991, and 1999) required multiple attempts to obtain a clone absent of mutations. (FIG. 1C) Diagram showing the typical mutations observed in the clones containing the 1983, 1991, 1999 avian N1 gene segments. (FIG. 1D) Agarose gel (0.8%) image of the PCR amplified pHW plasmid and the N1₉₈ and N1₉₉ gene segment inserts. (FIG. 1E) Representative images of the E. coli colonies that were obtained following transformation with the pHW plasmid and the indicated avian N1 gene segment insert. The higher magnification insets show the typical large (L) colony size observed for the pHW or pHW+N1₉₈ transformed bacteria along with the atypical smaller(S) colony size of the pHW+N1₉₉ transformed bacteria. Images are of the LB agar plates and the scale bars (white) correspond to 1 cm. (FIG. 1F) Agarose gels displaying the PCR screening results of 10 randomly selected colonies from each transformation. Bands corresponding to the pHW N1₉₈ and N1₉₉ positive clones, those not of the expected size (*) and target-independent PCR products (φ) observed from the empty pHW plasmid are indicated. NA genes in the positive clones were verified by sequencing. The data in FIGS. 1E and 1F are representative of three biological repeats.

FIGS. 2A-2B: Analysis of gene expression from the pHW plasmid in E. coli. (FIG. 2A) Schematic showing the gene expression analysis for the pHW plasmid in E. coli. Bacteria transformed with the pHW plasmid containing either the N1₉₈ or superfolder GFP (sfGFP) genes were grown overnight, lysed and analyzed by fluorescent size exclusion chromatography (FSEC) to detect sfGFP expression. (FIG. 2B) Representative FSEC chromatograms of lysates from E. coli transformed with the pHW plasmid containing either the N1₉₈ or sfGFP genes are displayed. The peak corresponding to sfGFP is indicated. Arrows indicate the elution volumes of the depicted molecular weight standards.

FIGS. 3A-3E: Analysis of gene expression from the pHW variant plasmids in prokaryotic and eukaryotic cells. (FIG. 3A) Diagrams showing how plasmid derived gene transcription can be minimized by the positioning of (i) the three cooperative wild type lac operator sequences or (ii) the E. coli rrnB transcriptional terminator around a gene of interest. (FIG. 3B) Schematics of the pHW plasmid variants with one carrying the three lac operator sequences (pHW/O₁₂₃) around sfGFP and the CMV promoter (SEQ ID NO: 6), the second containing the rrnB transcriptional terminator (pHW/T₁T₂), and the third containing both the lac operators and the terminator (pHW/O₁₂₃T₁T₂) (SEQ ID NO:7). (FIG. 3C) FSEC chromatograms of lysates from E. coli carrying the indicated pHW plasmid variants. The peak corresponding to sfGFP is indicated. The FSEC data are representative of two biological and three technical repeats. (FIG. 3D) GFP fluorescence of lysates prepared from 293-T cells transfected with the indicated pHW plasmid variant are shown. (FIG. 3E) Representative images showing GPP fluorescence in 293-T cells transfected with the indicated pHW plasmid variants. The insets show a brightfield image of the confluent cell layer.

FIGS. 4A-4D: Stability of the avian N1₉₉ gene segment in the pHW plasmid variants. (FIG. 4A) Schematic of the avian N1 gene segments with their 5′ and 3′ UTRs that were cloned into the indicated pHW plasmid variants. Shown are pHW (no operator or terminator sequences), pHW/O₁₂₃ (three operator sequences; SEQ ID NO: 11), pHW/T₁T₂ (terminator sequence only), and pHW/O₁₂₃T₁T₂ (three operator sequences and a terminator sequence; SEQ ID NO: 12). (FIG. 4B) Agarose gel (0.8%) of the PCR amplified pHW plasmid variants, and the avian N1₉₈ and N1₉₉ gene segments. (FIG. 4C) Representative images of the E. coli colonies that were obtained following transformation with the indicated pHW plasmid variant and avian N1 gene segment insert. The higher magnification insets show the typical large (L) colony size observed for the pHW+N1₉₈ transformed bacteria along with the large (L) and small(S) colony sizes observed for the bacteria transformed with the indicated pHW plasmid variant containing the avian N1₉₉ gene segment. Images are of the LB agar plates and the scale bars (white) correspond to 1 cm. (FIG. 4D) Representative agarose gels displaying the PCR screening results of 10 randomly selected colonies from each transformation. Bands corresponding to the appropriate size of the N1₉₈ and N1₉₉ genes are indicated. Asterisks denote bands that are not of the expected size. NA genes in the positive clones were verified by sequencing. The data in FIGS. 4C and 4D are representative of three biological repeats.

FIGS. 5A-5D: Stability of HA (H1 and H6) gene segments in the pHW plasmid variants. (FIG. 5A) Schematic of the two HA gene segments with their 5′ and 3′ UTRs that were cloned into the pHW and pHW/O₁₂₃ (SEQ ID NO: 8) plasmids. (FIG. 5B) Agarose gel (0.8%) image of the indicated PCR amplified pHW plasmids and HA gene segments. (FIG. 5C) Representative images of the E. coli colonies that were obtained following transformation with the indicated pHW plasmid variant and HA gene segment insert. The higher magnification insets show the large (L) and small(S) colony sizes that were observed following transformation. Images are of the LB agar plates and the scale bars (white) correspond to 1 cm. (FIG. 5D) Agarose gel (0.8%) images displaying the PCR screening results of 10 randomly selected colonies from each transformation. Bands corresponding to the appropriate size of the H1 and H6 genes are indicated. Asterisks denote bands that are not of the expected size. The HA genes in the positive clones were verified by sequencing. The data in FIGS. 5C and 5D are representative of three biological repeats.

FIGS. 6A-6D: Location and number of lac operators is important for H6 gene segment stability in the pHW plasmid. (FIG. 6A) Schematic of the H6 gene segment with its 5′ and 3′ UTRs that was cloned into pHW plasmid variants containing different combinations of the three lac operators. Shown are pHW (no operator or terminator sequences), pHW/O₁₂₃ (three operator sequences; SEQ ID NO: 8), pHW/O₁₂ (two operator sequences flanking the H6 gene), pHW/O₁₃ (two operator sequences upstream of the H6 gene), and pHW/O₃ (one operator sequence upstream of the promoter and H6 gene). (FIG. 6B) Representative images of theE. coli colonies that were obtained following transformation with the indicated pHW plasmid variant containing the H6 gene segment insert. The higher magnification insets show the large (L) and small(S) colony sizes that were observed. Images are of the LB agar plates and the scale bars (white) correspond to 1 cm. (FIG. 6C) Agarose gel displaying the PCR screening results of five pooled L and S colonies from each transformation. Bands corresponding to the appropriate size of the H6 gene segment are indicated. The asterisk denotes bands that are not of the expected size. (FIG. 6D) Representative images of the E. coli colonies that were obtained following transformation with the indicated plasmids and grown on LB agar plates with and without isopropyl β-D-1-thiogalactopyranoside (IPTG). The higher magnification insets show the L colonies observed following transformed with pHW+N1₉₈ (+/−IPTG) and the mostly smaller(S) colonies observed for the bacteria transformed with the pHW/O₁₂₃+H6 plasmid that was grown on LB agar plates lacking IPTG. Scale bars (white) correspond to 1 cm. The data in FIGS. 6C and 6D are representative of three biological repeats.

FIGS. 7A-7F: Influenza viruses can be rescued using the pHW/O₁₂₃ plasmid containing NA or HA gene segments. (FIG. 7A) Graphs displaying NA activity and hemagglutination unit (HAU) titers obtained for the indicated viruses during the reverse genetics rescue. NA activities and HAU titers were measured using equal volumes of cell culture supernatant collected at the indicated times post-transfection. The asterisks (*) indicate viruses (WSN^{N1/99}* and WSN^{H6 N1/18}*) generated with the pHW/O₁₂₃N1₉₉ and the pHW/O₁₂₃H6 plasmids respectively. The hashtag (#) represents a virus (WSN^{H6 N1/18 #}) generated with an independent commercial preparation of the pHW/O₁₂₃-H6 plasmid. (FIG. 7B) Graphs displaying NA activities and HAU titers obtained for the indicated viruses following the initial passage in eggs. The NA activities and HAU titers were measured using an equal volume of allantoic fluid from each egg at three days post-infection. Individual egg data is displayed with the mean (bar). P values were calculated from a two-tailed unpaired t-test. (FIG. 7C) Image of a Coomassie stained 4-12% SDS-PAGE gel containing 5 μg of the indicated virions isolated by sedimentation. Oxidized (OX) forms of the NA and HA proteins are indicated along with viral proteins NP and M1. (FIG. 7D) NA activities and HAU titers of the indicated viruses during reverse genetics (RG) rescue are shown. Measurements were from equal cell culture supernatant volumes. Asterisks denote viruses generated with eight pHW/O₁₂₃ plasmids (WSN*) or the pHW/O₁₂₃-N1₉₉ plasmid combined with seven PR8 pHW plasmids (PR8^{N1/99}*). (FIG. 7E) Viruses were passaged in eggs for 72 hours, and the HAU titers were measured from equal allantoic fluid volumes. Data from uninfected eggs were excluded. Each bar corresponds to the mean. (FIG. 7F) Nonreduced Coomassie-stained SDS-PAGE gel image of the indicated virions (˜5 μg) isolated by sedimentation. All P values were calculated from a two-tailed unpaired t-test (95% CI).

FIGS. 8A-8C: Representative sequence chromatograms of NA (N1) genes difficult to clone into pHW. PCR positive colonies containing pHW with the indicated N1 gene were grown overnight, plasmid DNA was isolated and analyzed by Sanger sequencing. Regions of sequence chromatograms showing insertions (FIG. 8A; SEQ ID NO: 23), point mutations (FIG. 8B; SEQ ID NO: 24) and the presence of mixed template (FIG. 8C; SEQ ID NO: 25) from the propagation of a single colony are displayed. N1 amino acids corresponding to each codon are displayed and the resulting substitutions are depicted in red. Ambiguous sequence and the N1 stop codon are indicated by dashes and an asterisk, respectively.

FIG. 9: Positioning of the lac operators and rrnB gene terminator in pHW/O₁₂₃T₁T₂. Nucleotide sequence of pHW/O₁₂₃T₁T₂ showing the sequence and positioning of the three lac operators (O₁, O₂, and O₃) and the rrnB terminator (T₁T₂) with respect to the CMV Pol II promoter, IAV gene insertion site (indicated by the IAV 5′ and 3′ UTRs) and the Pol I promoter. In this sequence (nucleotides 413-1858 of SEQ ID NO: 7), the inserted gene encoding sfGFP flanked by the IAV UTRs from HA, is situated between the O₁ and O₂ operators. The remaining pHW sequence is indicated by the dashed lines. Sequences of pHWO₁₂₃ and other pHW variants can be deduced by extracting the nucleotides of the transcriptional regulatory elements that are not present.

FIG. 10: Sequence chromatograms of the HA (H6) gene that is difficult to clone into pHW. Plasmid DNA isolated from pHW+H6 transformed E. coli culture was analyzed by Sanger sequencing. A schematic displaying the point of insertion is shown together with the sequence chromatograms (left, SEQ ID NO: 26; right, SEQ ID NO: 27). HA amino acids corresponding to each codon are shown with the resulting substitutions due to the insertion.

FIGS. 11A-11B: Exemplary recombinant nucleic acid molecules and plasmids for expression of toxic DNA sequences in E. coli. (FIG. 11A) Schematic of exemplary recombinant nucleic acid molecules, which can be cloned into a plasmid by ligation. All exemplary recombinant nucleic acid molecules include a first lac operator sequence located at position O1, and a second lac operator sequence located at position O2; O1 and O2 flank a heterologous DNA, as shown in (i). Optional components represented in (ii) to (v) include a first promoter upstream of O1 and the heterologous DNA sequence, a second promoter downstream of the heterologous DNA and O2, a third lac operator sequence located at position O3 (5′ of the first promoter), a fourth lac operator sequence located at position O4 (3′ of the second promoter), and a terminator sequence (T1/T2) positioned between O1 and the heterologous DNA. (FIG. 11B) Schematic of exemplary recombinant plasmids that can be used for cloning a toxic heterologous DNA. All exemplary recombinant plasmids include a first lac operator sequence located at position O1, and a second lac operator sequence located at position O2; O1 and O2 flank a multiple cloning site (MCS), as shown in (i). Optional components represented in (ii) to (v) include a first promoter upstream of O1 and the MCS, a second promoter downstream of the MCS and O2, a third lac operator sequence located at position O3 (5′ of the first promoter), a fourth lac operator sequence located at position O4 (3′ of the second promoter), and a terminator sequence (T1/T2) positioned between O1 and the MCS. Using appropriate restriction enzymes, a heterologous DNA can be cloned into a recombinant plasmid at the MCS. For FIGS. 11A-11B, O1, O2, O3 and O4 represent first, second, third and fourth (respectively) positions where operator sequences are present, but do not represent specific nucleic acid sequences (e.g., the operator sequence at position O1 can have the same sequence as the operator sequence at position O2, or the sequences can be different).

FIGS. 12A-12B: Plasmid maps. (FIG. 12A) Map of plasmid pHWO₁₂₃-sfGFP (SEQ ID NO: 6), which contains three lac operator sequences (labelled O1, O2 and O3) and a gene of interest (sfGFP). The first and second lac operator sequences (O1 and O2) flank the gene of interest and the third lac operator sequence (O3) is located 5′ of the first promoter. (FIG. 12B) Map of plasmid pHWO₁₂₃T₁T₂-sfGFP (SEQ ID NO: 7), which contains three lac operator sequences (labelled O1, O2 and O3), a terminator sequence (T1-T2) and a gene of interest. The first and second lac operator sequences (O1 and O2) flank the gene of interest, the third lac operator sequence (O3) is located 5′ of the first promoter, and the terminator sequence is located between O1 and the gene of interest.

FIGS. 13A-13C: H6 gene segment stability in pHW and pHWO₁₂₃ following re-transformation. (FIG. 13A) Agarose gel (0.8%) image of the PCR-amplified H6 gene segment from the sequence-verified H6 pHW and H6 pHW/O₁₂₃ plasmids that were used for E. coli transformation. (FIG. 13B) Representative images of E. coli colonies that were obtained on LB-agar Amp plates following transformation with the sequence and PCR verified H6 pHW and H6 pHW/O₁₂₃ plasmids. Insets show higher magnification of the large (L) and small(S) colony sizes that were observed. Scale bars correspond to 1 cm. (FIG. 13C) Agarose gel (0.8%) images of the PCR screening results from five randomly selected L and S colonies from each LB-agar Amp plate. Bands corresponding to the predicted H6 gene size are indicated. Asterisks denote a band that is not of the expected size.

FIGS. 14A-14C: Expression of genes placed between two operators is inducible in E. coli. (FIG. 14A) Diagram of the bacterial expression plasmid with the nucleoprotein (NP) influenza gene inserted between two operator sequences (O₁ and O₂). (FIG. 14B) Coomassie stained gel showing the expression of four NP variants following the addition of 0.4 mM IPTG for the indicated times. Equal volumes of E. coli were sedimented and lysed by sonication, and sample amounts were adjusted for biomass as follows: 15 μl, 10 μl and 4 μl were loaded for the 0-, 4-, and 18-hour samples, respectively. In FIG. 14B, * indicates N-terminal NP fusions and ** indicates C-terminal NP fusions. (FIG. 14C) Schematic illustrating two potential mechanisms by which the use of 5′ and 3′ flanking operators can silence gene expression in E. coli through LacI binding, which differs from commercial vectors that only use operators upstream of the 5′ region of the gene. Upon IPTG addition, LacI is released enabling transcription and translation to occur.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:
SEQ ID NO: 1 is the nucleotide sequence of lac operator 1 (O₁).

AATTGTGAGCGGATAACAATT

SEQ ID NO: 2 is the nucleotide sequence of lac operator 2 (O₂).

AAATGTGAGCGAGTAACAACC

SEQ ID NO: 3 is the nucleotide sequence of lac operator 3 (O₃).

GGCAGTGAGCGCAACGCAATT

SEQ ID NO: 4 is the nucleotide sequence of the rrnB T1/T2 terminator.

ATAAAACGAAAGGCTCAGTCGAAAGACTGGGCCTTTCGTTTTATCTGTT
GTTTGTCGGTGAACGCTCTCCTGAGTAGGACAAATCCGCCGGGAGCGGA
TTTGAACGTTGCGAAGCAACGGCCCGGAGGGTGGCGGGCAGGACGCCCG
CCATAAACTGCCAGGCATCAAATTAAGCAGAAGGCCATCCTGACGGATG
GCCTTTT

SEQ ID NO: 5 is an exemplary amino acid sequence of an E. coli Lac repressor monomer (residues that are part of the substrate binding pocket are shown in bold underline).

| | |
|---|---|
| 1 | MKPVTLYDVA EYAGVSYQTV SRVVNQASHV SAKTREKVEA AMAELNYIPN RVAQQLAGKQ |
| 61 | SLLIGVATSS LALHAPSQIV AAIKSRADQL GASVVVSMVE RSGVEACKAA VHNLLAQRVS |
| 121 | GLIINYPLDD KDATAVEAAC ANVPALFLDV SDQTPINSII FSHEDGTRLG VEHLVALGHQ |
| 181 | QIALLAGPLS SVSARLRLAG WHKYLTRNQI QPIAEREGDW SAMSGFQQTM QMLNEGIVPT |
| 241 | AMLVANDQMA LGAMRAITES GLRVGADISV VGYDDTEDSS CYIPPLTTIK QDFRLLGQTS |
| 301 | VDRLLQLSQG QAVKGNQLLP VSLVKRKTTL PPNTQTASPQ VLADSLMQLA RQISRLESGQ |

SEQ ID NO: 6 is the nucleotide sequence of an exemplary plasmid with three lac operator sequences and a heterologous DNA sequence encoding sfGFP (pHWO₁₂₃-sfGFP).

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGTGAG CGCAACGCAA TIgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TTGTGAGCGG ATAACAATTa gacccaagct gttaacgcta gcagttaacc |
| 781 | ggagtactgg tcgacctccg aagttggggg ggAGCAAAAG CAGGGGAAAA CAAAAGCAAC |
| 841 | AAAAATGAGC AAAGGAGAAG AACTTTTCAC TGGAGTTGTC CCAATTCTTG TTGAATTAGA |
| 901 | TGGTGATGTT AATGGGCACA AATTTTCTGT CAGAGGAGAG GGTGAAGGTG ATGCTACAAT |
| 961 | CGGAAAACTC ACCCTTAAAT TTATTTGCAC TACTGGAAAA CTACCTGTTC CATGGCCAAC |
| 1021 | ACTTGTCACT ACTCTGACCT ATGGTGTTCA ATGCTTTTCC CGTTATCCGG ATCACATGAA |
| 1081 | AAGGCATGAC TTTTTCAAGA GTGCCATGCC CGAAGGTTAT GTACAGGAAC GCACTATATC |
| 1141 | TTTCAAAGAT GACGGGAAAT ACAAGACGCG TGCTGTAGTC AAGTTTGAAG GTGATACCCT |
| 1201 | TGTTAATCGT ATCGAGTTAA AGGGTACTGA TTTTAAAGAA GATGGAAACA TTCTCGGACA |
| 1261 | CAAACTCGAG TACAACTTTA ACTCACACAA TGTATACATC ACGGCAGACA AACAAAAGAA |
| 1321 | TGGAATCAAA GCTAACTTCA CAGTTCGCCA CAACGTTGAA GATGGTTCCG TTCAACTAGC |
| 1381 | AGACCATTAT CAACAAAATA CTCCAATTGG CGATGGCCCT GTCCTTTTAC CAGACAACCA |
| 1441 | TTACCTGTCG ACACAAACTG TCCTTTCGAA AGATCCCAAC GAAAAGCGTG ACCACATGGT |
| 1501 | CCTTCATGAG TAtGTAAATG CTGCTGGGAT TACACATGGC ATGGATGAGC TCTACAAATA |
| 1561 | ACATTAGGAT TTCAGAATCA TGAGAAAAAC ACCCITGTTT CTACTaataa cccggcggcc |
| 1621 | caaaatgccg AAATGTGAGC GAGTAACAAC Cactcggagc gaaagatata cctcccccgg |
| 1681 | ggccgggagg tcgcgtcacc gaccacgccg ccggcccagg cgacgcgcga cacggacacc |
| 1741 | tgtccccaaa aacgccacca tcgcagccac acacggagcg cccggggccc tctggtcaac |
| 1801 | cccaggacac acgcgggagc agcgccgggc cggggacgcc ctcccggcgg tgacctggcc |
| 1861 | ctattctata gtgtcaccta aatgctagag ctcgctgatc agcctcgact gtgccttcta |
| 1921 | gttgccagcc atctgttgtt tgcccctccc ccgtgccttc cttgaccctg gaaggtgcca |
| 1981 | ctcccactgt cctttcctaa taaaatgagg aaattgcatc gcattgtctg agtaggtgtc |
| 2041 | attctattct ggggggtggg gtggggcagg acagcaaggg ggaggattgg gaagacaata |
| 2101 | gcaggcatgc tggggatgcg gtgggctcta tggcttctga ggcggaaaga accagctgca |
| 2161 | ttaatgaatc ggccaacgcg cggggagagg cggtttgcgt attgggcgct cttccgcttc |
| 2221 | ctcgctcact gactcgctgc gctcggtcgt tcggctgcgg cgagcggtat cagctcactc |
| 2281 | aaaggcggta atacggttat ccacagaatc aggggataac gcaggaaaga acatgtgagc |
| 2341 | aaaaggccag caaaaggcca ggaaccgtaa aaaggccgcg ttgctggcgt ttttccatag |
| 2401 | gctccgcccc cctgacgagc atcacaaaaa tcgacgctca agtcagaggt ggcgaaaccc |
| 2461 | gacaggacta taaagatacc aggcgtttcc ccctggaagc tccctcgtgc gctctcctgt |
| 2521 | tccgaccctg ccgcttaccg gatacctgtc cgcctttctc ccttcgggaa gcgtggcgct |
| 2581 | ttctcatagc tcacgctgta ggtatctcag ttcggtgtag gtcgttcgct ccaagctggg |
| 2641 | ctgtgtgcac gaaccccccg ttcagcccga ccgctgcgcc ttatccggta actatcgtct |
| 2701 | tgagtccaac ccggtaagac acgacttatc gccactggca gcagccactg gtaacaggat |
| 2761 | tagcagagcg aggtatgtag gcggtgctac agagttcttg aagtggtggc ctaactacgg |
| 2821 | ctacactaga agaacagtat ttggtatctg cgctctgctg aagccagtta ccttcggaaa |
| 2881 | aagagttggt agctcttgat ccggcaaaca aaccaccgct ggtagcggtg gtttttttgt |
| 2941 | ttgcaagcag cagattacgc gcagaaaaaa aggatctcaa gaagatcctt tgatcttttc |
| 3001 | tacggggtct gacgctcagt ggaacgaaaa ctcacgttaa gggattttgg tcatgagatt |
| 3061 | atcaaaaagg atcttcacct agatcctttt aaattaaaaa tgaagtttta aatcaatcta |
| 3121 | aagtatatat gagtaaactt ggtctgacag ttaccaatgc ttaatcagtg aggcacctat |
| 3181 | ctcagcgatc tgtctatttc gttcatccat agttgcctga ctccccgtcg tgtagataac |
| 3241 | tacgatacgg gagggcttac catctggccc cagtgctgca atgataccgc gagacccacg |
| 3301 | ctcaccggct ccagatttat cagcaataaa ccagccagcc ggaagggccg agcgcagaag |
| 3361 | tggtcctgca actttatccg cctccatcca gtctattaat tgttgccggg aagctagagt |
| 3421 | aagtagttcg ccagttaata gtttgcgcaa cgttgttgcc attgctacag gcatcgtggt |
| 3481 | gtcacgctcg tcgtttggta tggcttcatt cagctccggt tcccaacgat caaggcgagt |
| 3541 | tacatgatcc cccatgttgt gcaaaaaagc ggttagctcc ttcggtcctc cgatcgttgt |
| 3601 | cagaagtaag ttggccgcag tgttatcact catggttatg gcagcactgc ataattctct |
| 3661 | tactgtcatg ccatccgtaa gatgcttttc tgtgactggt gagtactcaa ccaagtcatt |
| 3721 | ctgagaatag tgtatgcggc gaccgagttg ctcttgcccg gcgtcaatac gggataatac |
| 3781 | cgcgccacat agcagaactt taaaagtgct catcattgga aaacgttctt cggggcgaaa |

SEQ ID NO: 7 is the nucleotide sequence of an exemplary plasmid with three lac operator sequences, a terminator sequence and a heterologous DNA sequence encoding sfGFP (pHWO₁₂₃T₁T₂-sfGFP).

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGTGAG CGCAACGCAA TTgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TTGTGAGCGG ATAACAATTa gacccaagct gttaacgATA AAACGAAAGG |
| 781 | CTCAGTCGAA AGACTGGGCC TTTCGTTTTA TCTGTTGTTT GTCGGTGAAC GCTCTCCTGA |
| 841 | GTAGGACAAA TCCGCCGGGA GCGGATTTGA ACGTTGCGAA GCAACGGCCC GGAGGGTGGC |
| 901 | GGGCAGGACG CCCGCCATAA ACTGCCAGGC ATCAAATTAA GCAGAAGGCC ATCCTGACGG |
| 961 | ATGGCCTTTT ctagcagtta accggagtac tggtcgacct ccgaagttgg gggggAGCAA |
| 1021 | AAGCAGGGGA AAACAAAAGC AACAAAAATG AGCAAAGGAG AAGAACTTTT CACTGGAGTT |
| 1081 | GTCCCAATTC TTGTTGAATT AGATGGTGAT GTTAATGGGC ACAAATTTTC TGTCAGAGGA |
| 1141 | GAGGGTGAAG GTGATGCTAC AATCGGAAAA CTCACCCTTA AATTTATTTG CACTACTGGA |
| 1201 | AAACTACCTG TTCCATGGCC AACACTTGTC ACTACTCTGA CCTATGGTGT TCAATGCTTT |
| 1261 | TCCCGTTATC CGGATCACAT GAAAAGGCAT GACTTTTTCA AGAGTGCCAT GCCCGAAGGT |
| 1321 | TATGTACAGG AACGCACTAT ATCTTTCAAA GATGACGGGA AATACAAGAC GCGTGCTGTA |
| 1381 | GTCAAGTTTG AAGGTGATAC CCTTGTTAAT CGTATCGAGT TAAAGGGTAC TGATTTTAAA |
| 1441 | GAAGATGGAA ACATTCTCGG ACACAAACTC GAGTACAACT TTAACTCACA CAATGTATAC |
| 1501 | ATCACGGCAG ACAAACAAAA GAATGGAATC AAAGCTAACT TCACAGTTCG CCACAACGTT |
| 1561 | GAAGATGGTT CCGTTCAACT AGCAGACCAT TATCAACAAA ATACTCCAAT TGGCGATGGC |
| 1621 | CCTGTCCTTT TACCAGACAA CCATTACCTG TCGACACAAA CTGTCCTTTC GAAAGATCCC |
| 1681 | AACGAAAAGC GTGACCACAT GGTCCTTCAT GAGTAtGTAA ATGCTGCTGG GATTACACAT |
| 1741 | GGCATGGATG AGCTCTACAA ATAACATTAG GATTICAGAA TCATGAGAAA AACACCCTTG |
| 1801 | TTTCTACTaa taacccggcg gcccaaaatg ccgAAATGTG AGCGAGTAAC AACCactcgg |
| 1861 | agcgaaagat atacctcccc cggggccggg aggtcgcgtc accgaccacg ccgccggccc |
| 1921 | aggcgacgcg cgacacggac acctgtcccc aaaaacgcca ccatcgcagc cacacacgga |
| 1981 | gcgcccgggg ccctctggtc aaccccagga cacacgcggg agcagcgccg ggccggggac |
| 2041 | gccctcccgg cggtgacctg gccctattct atagtgtcac ctaaatgcta gagctcgctg |
| 2101 | atcagcctcg actgtgcctt ctagttgcca gccatctgtt gtttgcccct cccccgtgcc |
| 2161 | ttccttgacc ctggaaggtg ccactcccac tatcctttcc taataaaatg aggaaattgc |
| 2221 | atcgcattgt ctgagtaggt gtcattctat tctggggggt ggggtggggc aggacagcaa |
| 2281 | gggggaggat tgggaagaca atagcaggca tgctggggat gcggtgggct ctatggcttc |
| 2341 | tgaggcggaa agaaccagct gcattaatga atcggccaac gcgcggggag aggcggtttg |
| 2401 | cgtattgggc gctcttccgc ttcctcgctc actgactcgc tgcgctcggt cgttcggctg |
| 2461 | cggcgagcgg tatcagctca ctcaaaggcg gtaatacggt tatccacaga atcaggggat |
| 2521 | aacgcaggaa agaacatgtg agcaaaaggc cagcaaaagg ccaggaaccg taaaaaggcc |
| 2581 | gcgttgctgg cgtttttcca taggctccgc ccccctgacg agcatcacaa aaatcgacgc |
| 2641 | tcaagtcaga ggtggcgaaa cccgacagga ctataaagat accaggcgtt tccccctgga |
| 2701 | agctccctcg tgcgctctcc tgttccgacc ctgccgctta ccggatacct gtccgccttt |
| 2761 | ctcccttcgg gaagcgtggc gctttctcat agctcacgct gtaggtatct cagttcggtg |
| 2821 | taggtcgttc gctccaagct gggctgtgtg cacgaacccc ccgttcagcc cgaccgctgc |
| 2881 | gccttatccg gtaactatcg tcttgagtcc aacccggtaa gacacgactt atcgccactg |
| 2941 | gcagcagcca ctggtaacag gattagcaga gcgaggtatg taggcggtgc tacagagttc |
| 3001 | ttgaagtggt ggcctaacta cggctacact agaagaacag tatttggtat ctgcgctctg |
| 3061 | ctgaagccag ttaccttcgg aaaaagagtt ggtagctctt gatccggcaa acaaaccacc |
| 3121 | gctggtagcg gtggtttttt tgtttgcaag cagcagatta cgcgcagaaa aaaaggatct |
| 3181 | caagaagatc ctttgatctt ttctacgggg tctgacgctc agtggaacga aaactcacgt |
| 3241 | taagggattt tggtcatgag attatcaaaa aggatcttca cctagatcct tttaaattaa |
| 3301 | aaatgaagtt ttaaatcaat ctaaagtata tatgagtaaa cttggtctga cagttaccaa |
| 3361 | tgcttaatca gtgaggcacc tatctcagcg atctgtctat ttcgttcatc catagttgcc |
| 3421 | tgactccccg tcgtgtagat aactacgata cgggagggct taccatctgg ccccagtgct |
| 3481 | gcaatgatac cgcgagaccc acgctcaccg gctccagatt tatcagcaat aaaccagcca |
| 3541 | gccggaaggg ccgagcgcag aagtggtcct gcaactttat ccgcctccat ccagtctatt |
| 3601 | aattgttgcc gggaagctag agtaagtagt tcgccagtta atagtttgcg caacgttgtt |
| 3661 | gccattgcta caggcatcgt ggtgtcacgc tcgtcgtttg gtatggcttc attcagctcc |
| 3721 | ggttcccaac gatcaaggcg agttacatga tcccccatgt tgtgcaaaaa agcggttagc |
| 3781 | tccttcggtc ctccgatcgt tgtcagaagt aagttggccg cagtgttatc actcatggtt |
| 3841 | atggcagcac tgcataattc tcttactgtc atgccatccg taagatgctt ttctgtgact |
| 3901 | ggtgagtact caaccaagtc attctgagaa tagtgtatgc ggcgaccgag ttgctcttgc |
| 3961 | ccggcgtcaa tacgggataa taccgcgcca catagcagaa ctttaaaagt gctcatcatt |
| 4021 | ggaaaacgtt cttcggggcg aaa |

SEQ ID NO: 8 is the nucleotide sequence of an exemplary plasmid with three lac operator sequences and a heterologous DNA sequence encoding an influenza virus HA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGIGAG CGCAACGCAA TTgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TIGTGAGCGG ATAACAATTa gacccaagct gttaacgcta gcagttaacc |
| 781 | ggagtactgg tcgacctccg aagttggggg ggagcaaaag caggggaaaA TGattgcaat |
| 841 | cataataatc gcggtagtgg cctctaccag caaatcagac aagatctgca ttgggtatca |
| 901 | tgccaacaac tcgacaacac aagtggacac aatattagag aagaatgtga cagtgacgca |
| 961 | ctctgtagag ctcctagaaa gtcagaagga ggagagattc tgcagagtgt tgaataaaac |
| 1021 | acctctggat ctaaagggtt gcaccattga aggatggatt cttggaaacc cccaatgtga |
| 1081 | catcttactt ggtgaccaaa gttggtcata catagtagag aggcctggag cccaaaatgg |
| 1141 | gatatgttac ccaggggtgc tgaacgaagt ggaagaactg aaagcattca ttgggtccgg |
| 1201 | agagaaagta cagagatttg aaatgtttcc caagagcacg tggaccggag tggacactaa |
| 1261 | cagtggagtt acgagagctt gcccctatac taccagtgga tcatcctttt acaggaatct |
| 1321 | tttgtggata ataaaaacaa ggtctgctgc atacccagta attaagggaa catacaataa |
| 1381 | tactggctcc cagccaatcc tatatttctg gggtgtgcat catcctccaa ataccgatga |
| 1441 | gcaaaatacc ttatatggct ctggtgacag gtatgttaga atgggaactg aaagcatgaa |
| 1501 | ttttgccaag agtcctgaaa tagcagccag gccagctgtg aatgggcaaa gaggaagaat |
| 1561 | tgattattat tggtctgtac tgaaaccagg agaaacctta aatgtagaat ccaatggaaa |
| 1621 | tttaatagct ccttggtatg cttacaagtt cacaagttcc aacaacaaag gagctatctt |
| 1681 | caaatcaaac ctcccaattg agaattgtga tgctgtatgt caaactgttg ctggagcact |
| 1741 | aaagacaaac aaaactttcc aaaatgttag tccactctgg attggagaat gtcccaaata |
| 1801 | tgttaagagt gagagcctaa gactggcaac tggtctgagg aatgtcccac aggcagaaac |
| 1861 | aagaggattg tttggagcca tagctgggtt tatagaagga gggtggacag gtatgataga |
| 1921 | cggatggtac gggtaccatc atgagaactc acaggggtcg ggttatgcag cagataaaga |
| 1981 | aagtacccag aaagcaattg acgggatcac caataaagta aattccatca ttgacaagat |
| 2041 | gaacacacag tttgaagcag tagagcatga gttctcaaat ctcgaaagga gaatagacaa |
| 2101 | tttaaacaaa agaatggaag atggattttt ggatgtgtgg acgtacaatg ctgaactttt |
| 2161 | agttctactg gaaaatgaaa ggaccctgga tctgcacgat gccaatgtga agaacctata |
| 2221 | cgagaaggtg aaatcacaat tgagagataa tgcaaaggat ttgggtaatg ggtgttttga |
| 2281 | attttggcac aaatgcgacg atgaatgcat caactcagtt aagaatggca catacgatta |
| 2341 | cccaaagtac caagacgaga gcaaacttaa cagacaggag atagactcag tgaagctgga |
| 2401 | aaatctgggc gtatatcaaa ttcttgctat ttatagtacg gtatcgagca gtctagtttt |
| 2461 | ggtggggctg atcattgcca tgggtctttg gatgtgctca aatggctcaa tgcaatgcag |
| 2521 | gatatgtata TAAttagaaa aaaacaccct tgtttctact aataacccgg cggcccaaaa |
| 2581 | tgccgAAATG TGAGCGAGTA ACAACCactc ggagcgaaag atatacctcc cccggggccg |
| 2641 | ggaggtcgcg tcaccgacca cgccgccggc ccaggcgacg cgcgacacgg acacctgtcc |
| 2701 | ccaaaaacgc caccatcgca gccacacacg gagcgcccgg ggccctctgg tcaaccccag |
| 2761 | gacacacgcg ggagcagcgc cgggccgggg acgccctccc ggcggtgacc tggccctatt |
| 2821 | ctatagtgtc acctaaatgc tagagctcgc tgatcagcct cgactgtgcc ttctagttgc |
| 2881 | cagccatctg ttgtttgccc ctcccccgtg ccttccttga ccctggaagg tgccactccc |
| 2941 | actgtccttt cctaataaaa tgaggaaatt gcatcgcatt gtctgagtag gtgtcattct |
| 3001 | attctggggg gtggggtggg gcaggacagc aagggggagg attgggaaga caatagcagg |
| 3061 | catgctgggg atgcggtggg ctctatggct tctgaggcgg aaagaaccag ctgcattaat |
| 3121 | gaatcggcca acgcgcgggg agaggcggtt tgcgtattgg gcgctcttcc gcttcctcgc |
| 3181 | tcactgactc gctgcgctcg gtcgttcggc tgcggcgagc ggtatcagct cactcaaagg |
| 3241 | cggtaatacg gttatccaca gaatcagggg ataacgcagg aaagaacatg tgagcaaaag |
| 3301 | gccagcaaaa ggccaggaac cgtaaaaagg ccgcgttgct ggcgtttttc cataggctcc |
| 3361 | gcccccctga cgagcatcac aaaaatcgac gctcaagtca gaggtggcga aacccgacag |
| 3421 | gactataaag ataccaggcg tttccccctg gaagctccct cgtgcgctct cctgttccga |
| 3481 | ccctgccgct taccggatac ctgtccgcct ttctcccttc gggaagcgtg gcgctttctc |
| 3541 | atagctcacg ctgtaggtat ctcagttcgg tgtaggtcgt tcgctccaag ctgggctgtg |
| 3601 | tgcacgaacc ccccgttcag cccgaccgct gcgccttatc cggtaactat cgtcttgagt |
| 3661 | ccaacccggt aagacacgac ttatcgccac tggcagcagc cactggtaac aggattagca |
| 3721 | gagcgaggta tgtaggcggt gctacagagt tcttgaagtg gtggcctaac tacggctaca |
| 3781 | ctagaagaac agtatttggt atctgcgctc tgctgaagcc agttaccttc ggaaaaagag |
| 3841 | ttggtagctc ttgatccggc aaacaaacca ccgctggtag cggtggtttt tttgtttgca |
| 3901 | agcagcagat tacgcgcaga aaaaaaggat ctcaagaaga tcctttgatc ttttctacgg |
| 3961 | ggtctgacgc tcagtggaac gaaaactcac gttaagggat tttggtcatg agattatcaa |
| 4021 | aaaggatctt cacctagatc cttttaaatt aaaaatgaag ttttaaatca atctaaagta |
| 4081 | tatatgagta aacttggtct gacagttacc aatgcttaat cagtgaggca cctatctcag |
| 4141 | cgatctgtct atttcgttca tccatagttg cctgactccc cgtcgtgtag ataactacga |
| 4201 | tacgggaggg cttaccatct ggccccagtg ctgcaatgat accgcgagac ccacgctcac |
| 4261 | cggctccaga tttatcagca ataaaccagc cagccggaag ggccgagcgc agaagtggtc |
| 4321 | ctgcaacttt atccgcctcc atccagtcta ttaattgttg ccgggaagct agagtaagta |
| 4381 | gttcgccagt taatagtttg cgcaacgttg ttgccattgc tacaggcatc gtggtgtcac |
| 4441 | gctcgtcgtt tggtatggct tcattcagct ccggttccca acgatcaagg cgagttacat |
| 4501 | gatcccccat gttgtgcaaa aaagcggtta gctccttcgg tcctccgatc gttgtcagaa |
| 4561 | gtaagttggc cgcagtgtta tcactcatgg ttatggcagc actgcataat tctcttactg |
| 4621 | tcatgccatc cgtaagatgc ttttctgtga ctggtgagta ctcaaccaag tcattctgag |
| 4681 | aatagtgtat gcggcgaccg agttgctctt gcccggcgtc aatacgggat aataccgcgc |
| 4741 | cacatagcag aactttaaaa gtgctcatca ttggaaaacg ttcttcgggg cgaaa |

SEQ ID NO: 9 is the nucleotide sequence of an exemplary plasmid with three lac operator sequences, a terminator sequence and a heterologous DNA sequence encoding an influenza virus HA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGTGAG CGCAACGCAA TTgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TIGTGAGCGG ATAACAATTa gacccaagct gttaacgATA AAACGAAAGG |
| 781 | CTCAGTCGAA AGACTGGGCC TTTCGTTITA TCTGTTGTTT GTCGGTGAAC GCTCTCCTGA |
| 841 | GTAGGACAAA TCCGCCGGGA GCGGATTTGA ACGTTGCGAA GCAACGGCCC GGAGGGTGGC |
| 901 | GGGCAGGACG CCCGCCATAA ACTGCCAGGC ATCAAATTAA GCAGAAGGCC ATCCTGACGG |
| 961 | ATGGCCTTTT ctagcagtta accggagtac tggtcgacct ccgaagttgg gggggagcaa |
| 1021 | aagcagggga aaATGattgc aatcataata atcgcggtag tggcctctac cagcaaatca |
| 1081 | gacaagatct gcattgggta tcatgccaac aactcgacaa cacaagtgga cacaatatta |
| 1141 | gagaagaatg tgacagtgac gcactctgta gagctcctag aaagtcagaa ggaggagaga |
| 1201 | ttctgcagag tgttgaataa aacacctctg gatctaaagg gttgcaccat tgaaggatgg |
| 1261 | attcttggaa acccccaatg tgacatctta cttggtgacc aaagttggtc atacatagta |
| 1321 | gagaggcctg gagcccaaaa tgggatatgt tacccagggg tgctgaacga agtggaagaa |
| 1381 | ctgaaagcat tcattgggtc cggagagaaa gtacagagat ttgaaatgtt tcccaagagc |
| 1441 | acgtggaccg gagtggacac taacagtgga gttacgagag cttgccccta tactaccagt |
| 1501 | ggatcatcct tttacaggaa tcttttgtgg ataataaaaa caaggtctgc tgcataccca |
| 1561 | gtaattaagg gaacatacaa taatactggc tcccagccaa tcctatattt ctggggtgtg |
| 1621 | catcatcctc caaataccga tgagcaaaat accttatatg gctctggtga caggtatgtt |
| 1681 | agaatgggaa ctgaaagcat gaattttgcc aagagtcctg aaatagcagc caggccagct |
| 1741 | gtgaatgggc aaagaggaag aattgattat tattggtctg tactgaaacc aggagaaacc |
| 1801 | ttaaatgtag aatccaatgg aaatttaata gctccttggt atgcttacaa gttcacaagt |
| 1861 | tccaacaaca aaggagctat cttcaaatca aacctcccaa ttgagaattg tgatgctgta |
| 1921 | tgtcaaactg ttgctggagc actaaagaca aacaaaactt tccaaaatgt tagtccactc |
| 1981 | tggattggag aatgtcccaa atatgttaag agtgagagcc taagactggc aactggtctg |
| 2041 | aggaatgtcc cacaggcaga aacaagagga ttgtttggag ccatagctgg gtttatagaa |
| 2101 | ggagggtgga caggtatgat agacggatgg tacgggtacc atcatgagaa ctcacagggg |
| 2161 | tcgggttatg cagcagataa agaaagtacc cagaaagcaa ttgacgggat caccaataaa |
| 2221 | gtaaattcca tcattgacaa gatgaacaca cagtttgaag cagtagagca tgagttctca |
| 2281 | aatctcgaaa ggagaataga caatttaaac aaaagaatgg aagatggatt tttggatgtg |
| 2341 | tggacgtaca atgctgaact tttagttcta ctggaaaatg aaaggaccct ggatctgcac |
| 2401 | gatgccaatg tgaagaacct atacgagaag gtgaaatcac aattgagaga taatgcaaag |
| 2461 | gatttgggta atgggtgttt tgaattttgg cacaaatgcg acgatgaatg catcaactca |
| 2521 | gttaagaatg gcacatacga ttacccaaag taccaagacg agagcaaact taacagacag |
| 2581 | gagatagact cagtgaagct ggaaaatctg ggcgtatatc aaattcttgc tatttatagt |
| 2641 | acggtatcga gcagtctagt tttggtgggg ctgatcattg ccatgggtct ttggatgtgc |
| 2701 | tcaaatggct caatgcaatg caggatatgt ataTAAttag aaaaaaacac ccttgtttct |
| 2761 | actaataacc cggcggccca aaatgccgAA ATGTGAGCGA GTAACAACCa ctcggagcga |
| 2821 | aagatatacc tcccccgggg ccgggaggtc gcgtcaccga ccacgccgcc ggcccaggcg |
| 2881 | acgcgcgaca cggacacctg tccccaaaaa cgccaccatc gcagccacac acggagcgcc |
| 2941 | cggggccctc tggtcaaccc caggacacac gcgggagcag cgccgggccg gggacgccct |
| 3001 | cccggcggtg acctggccct attctatagt gtcacctaaa tgctagagct cgctgatcag |
| 3061 | cctcgactgt gccttctagt tgccagccat ctgttgtttg cccctccccc gtgccttcct |
| 3121 | tgaccctgga aggtgccact cccactgtcc tttcctaata aaatgaggaa attgcatcgc |
| 3181 | attgtctgag taggtgtcat tctattctgg ggggtggggt ggggcaggac agcaaggggg |
| 3241 | aggattggga agacaatagc aggcatgctg gggatgcggt gggctctatg gcttctgagg |
| 3301 | cggaaagaac cagctgcatt aatgaatcgg ccaacgcgcg gggagaggcg gtttgcgtat |
| 3361 | tgggcgctct tccgcttcct cgctcactga ctcgctgcgc tcggtcgttc ggctgcggcg |
| 3421 | agcggtatca gctcactcaa aggcggtaat acggttatcc acagaatcag gggataacgc |
| 3481 | aggaaagaac atgtgagcaa aaggccagca aaaggccagg aaccgtaaaa aggccgcgtt |
| 3541 | gctggcgttt ttccataggc tccgcccccc tgacgagcat cacaaaaatc gacgctcaag |
| 3601 | tcagaggtgg cgaaacccga caggactata aagataccag gcgtttcccc ctggaagctc |
| 3661 | cctcgtgcgc tctcctgttc cgaccctgcc gcttaccgga tacctgtccg cctttctccc |
| 3721 | ttcgggaagc gtggcgcttt ctcatagctc acgctgtagg tatctcagtt cggtgtaggt |
| 3781 | cgttcgctcc aagctgggct gtgtgcacga accccccgtt cagcccgacc gctgcgcctt |
| 3841 | atccggtaac tatcgtcttg agtccaaccc ggtaagacac gacttatcgc cactggcagc |
| 3901 | agccactggt aacaggatta gcagagcgag gtatgtaggc ggtgctacag agttcttgaa |
| 3961 | gtggtggcct aactacggct acactagaag aacagtattt ggtatctgcg ctctgctgaa |
| 4021 | gccagttacc ttcggaaaaa gagttggtag ctcttgatcc ggcaaacaaa ccaccgctgg |
| 4081 | tagcggtggt ttttttgttt gcaagcagca gattacgcgc agaaaaaaag gatctcaaga |
| 4141 | agatcctttg atcttttcta cggggtctga cgctcagtgg aacgaaaact cacgttaagg |
| 4201 | gattttggtc atgagattat caaaaaggat cttcacctag atccttttaa attaaaaatg |
| 4261 | aagttttaaa tcaatctaaa gtatatatga gtaaacttgg tctgacagtt accaatgctt |
| 4321 | aatcagtgag gcacctatct cagcgatctg tctatttcgt tcatccatag ttgcctgact |
| 4381 | ccccgtcgtg tagataacta cgatacggga gggcttacca tctggcccca gtgctgcaat |
| 4441 | gataccgcga gacccacgct caccggctcc agatttatca gcaataaacc agccagccgg |
| 4501 | aagggccgag cgcagaagtg gtcctgcaac tttatccgcc tccatccagt ctattaattg |
| 4561 | ttgccgggaa gctagagtaa gtagttcgcc agttaatagt ttgcgcaacg ttgttgccat |
| 4621 | tgctacaggc atcgtggtgt cacgctcgtc gtttggtatg gcttcattca gctccggttc |
| 4681 | ccaacgatca aggcgagtta catgatcccc catgttgtgc aaaaaagcgg ttagctcctt |
| 4741 | cggtcctccg atcgttgtca gaagtaagtt ggccgcagtg ttatcactca tggttatggc |
| 4801 | agcactgcat aattctctta ctgtcatgcc atccgtaaga tgcttttctg tgactggtga |
| 4861 | gtactcaacc aagtcattct gagaatagtg tatgcggcga ccgagttgct cttgcccggc |
| 4921 | gtcaatacgg gataataccg cgccacatag cagaacttta aaagtgctca tcattggaaa |
| 4981 | acgttcttcg gggcgaaa |

SEQ ID NO: 10 is the nucleotide sequence of an exemplary plasmid with two lac operator sequences and a heterologous DNA sequence encoding an influenza virus HA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | agtcatcgct attaccatgg tgatgcggtt ttggcagtac atcaatgggc gtggatagcg |
| 481 | gtttgactca cggggatttc caagtctcca ccccattgac gtcaatggga gtttgttttg |
| 541 | gcaccaaaat caacgggact ttccaaaatg tcgtaacaac tccgccccat tgacgcaaat |
| 601 | gggcggtagg cgtgtacggt gggaggtcta tataagcaga gctctctggc taactagaga |
| 661 | acccactgct tactggctta tcgaaattaa tacgactcac tatagggAAT TGTGAGCGGA |
| 721 | TAACAATTag acccaagctg ttaacgctag cagttaaccg gagtactggt cgacctccga |
| 781 | agttgggggg gagcaaaagc aggggaaaAT Gattgcaatc ataataatcg cggtagtggc |
| 841 | ctctaccagc aaatcagaca agatctgcat tgggtatcat gccaacaact cgacaacaca |
| 901 | agtggacaca atattagaga agaatgtgac agtgacgcac tctgtagagc tcctagaaag |
| 961 | tcagaaggag gagagattct gcagagtgtt gaataaaaca cctctggatc taaagggttg |
| 1021 | caccattgaa ggatggattc ttggaaaccc ccaatgtgac atcttacttg gtgaccaaag |
| 1081 | ttggtcatac atagtagaga ggcctggagc ccaaaatggg atatgttacc caggggtgct |
| 1141 | gaacgaagtg gaagaactga aagcattcat tgggtccgga gagaaagtac agagatttga |
| 1201 | aatgtttccc aagagcacgt ggaccggagt ggacactaac agtggagtta cgagagcttg |
| 1261 | cccctatact accagtggat catcctttta caggaatctt ttgtggataa taaaaacaag |
| 1321 | gtctgctgca tacccagtaa ttaagggaac atacaataat actggctccc agccaatcct |
| 1381 | atatttctgg ggtgtgcatc atcctccaaa taccgatgag caaaatacct tatatggctc |
| 1441 | tggtgacagg tatgttagaa tgggaactga aagcatgaat tttgccaaga gtcctgaaat |
| 1501 | agcagccagg ccagctgtga atgggcaaag aggaagaatt gattattatt ggtctgtact |
| 1561 | gaaaccagga gaaaccttaa atgtagaatc caatggaaat ttaatagctc cttggtatgc |
| 1621 | ttacaagttc acaagttcca acaacaaagg agctatcttc aaatcaaacc tcccaattga |
| 1681 | gaattgtgat gctgtatgtc aaactgttgc tggagcacta aagacaaaca aaactttcca |
| 1741 | aaatgttagt ccactctgga ttggagaatg tcccaaatat gttaagagtg agagcctaag |
| 1801 | actggcaact ggtctgagga atgtcccaca ggcagaaaca agaggattgt ttggagccat |
| 1861 | agctgggttt atagaaggag ggtggacagg tatgatagac ggatggtacg ggtaccatca |
| 1921 | tgagaactca caggggtcgg gttatgcagc agataaagaa agtacccaga aagcaattga |
| 1981 | cgggatcacc aataaagtaa attccatcat tgacaagatg aacacacagt ttgaagcagt |
| 2041 | agagcatgag ttctcaaatc tcgaaaggag aatagacaat ttaaacaaaa gaatggaaga |
| 2101 | tggatttttg gatgtgtgga cgtacaatgc tgaactttta gttctactgg aaaatgaaag |
| 2161 | gaccctggat ctgcacgatg ccaatgtgaa gaacctatac gagaaggtga aatcacaatt |
| 2221 | gagagataat gcaaaggatt tgggtaatgg gtgttttgaa ttttggcaca aatgcgacga |
| 2281 | tgaatgcatc aactcagtta agaatggcac atacgattac ccaaagtacc aagacgagag |
| 2341 | caaacttaac agacaggaga tagactcagt gaagctggaa aatctgggcg tatatcaaat |
| 2401 | tcttgctatt tatagtacgg tatcgagcag tctagttttg gtggggctga tcattgccat |
| 2461 | gggtctttgg atgtgctcaa atggctcaat gcaatgcagg atatgtataT AAttagaaaa |
| 2521 | aaacaccctt gtttctacta ataacccggc ggcccaaaat gccgAAATGT GAGCGAGTAA |
| 2581 | CAACCactcg gagcgaaaga tatacctccc ccggggccgg gaggtcgcgt caccgaccac |
| 2641 | gccgccggcc caggcgacgc gcgacacgga cacctgtccc caaaaacgcc accatcgcag |
| 2701 | ccacacacgg agcgcccggg gccctctggt caaccccagg acacacgcgg gagcagcgcc |
| 2761 | gggccgggga cgccctcccg gcggtgacct ggccctattc tatagtgtca cctaaatgct |
| 2821 | agagctcgct gatcagcctc gactgtgcct tctagttgcc agccatctgt tgtttgcccc |
| 2881 | tcccccgtgc cttccttgac cctggaaggt gccactccca ctgtcctttc ctaataaaat |
| 2941 | gaggaaattg catcgcattg tctgagtagg tgtcattcta ttctgggggg tggggtgggg |
| 3001 | caggacagca agggggagga ttgggaagac aatagcaggc atgctgggga tgcggtgggc |
| 3061 | tctatggctt ctgaggcgga aagaaccagc tgcattaatg aatcggccaa cgcgcgggga |
| 3121 | gaggcggttt gcgtattggg cgctcttccg cttcctcgct cactgactcg ctgcgctcgg |
| 3181 | tcgttcggct gcggcgagcg gtatcagctc actcaaaggc ggtaatacgg ttatccacag |
| 3241 | aatcagggga taacgcagga aagaacatgt gagcaaaagg ccagcaaaag gccaggaacc |
| 3301 | gtaaaaaggc cgcgttgctg gcgtttttcc ataggctccg cccccctgac gagcatcaca |
| 3361 | aaaatcgacg ctcaagtcag aggtggcgaa acccgacagg actataaaga taccaggcgt |
| 3421 | ttccccctgg aagctccctc gtgcgctctc ctgttccgac cctgccgctt accggatacc |
| 3481 | tgtccgcctt tctcccttcg ggaagcgtgg cgctttctca tagctcacgc tgtaggtatc |
| 3541 | tcagttcggt gtaggtcgtt cgctccaagc tgggctgtgt gcacgaaccc cccgttcagc |
| 3601 | ccgaccgctg cgccttatcc ggtaactatc gtcttgagtc caacccggta agacacgact |
| 3661 | tatcgccact ggcagcagcc actggtaaca ggattagcag agcgaggtat gtaggcggtg |
| 3721 | ctacagagtt cttgaagtgg tggcctaact acggctacac tagaagaaca gtatttggta |
| 3781 | tctgcgctct gctgaagcca gttaccttcg gaaaaagagt tggtagctct tgatccggca |
| 3841 | aacaaaccac cgctggtagc ggtggttttt ttgtttgcaa gcagcagatt acgcgcagaa |
| 3901 | aaaaaggatc tcaagaagat cctttgatct tttctacggg gtctgacgct cagtggaacg |
| 3961 | aaaactcacg ttaagggatt ttggtcatga gattatcaaa aaggatcttc acctagatcc |
| 4021 | ttttaaatta aaaatgaagt tttaaatcaa tctaaagtat atatgagtaa acttggtctg |
| 4081 | acagttacca atgcttaatc agtgaggcac ctatctcagc gatctgtcta tttcgttcat |
| 4141 | ccatagttgc ctgactcccc gtcgtgtaga taactacgat acgggagggc ttaccatctg |
| 4201 | gccccagtgc tgcaatgata ccgcgagacc cacgctcacc ggctccagat ttatcagcaa |
| 4261 | taaaccagcc agccggaagg gccgagcgca gaagtggtcc tgcaacttta tccgcctcca |
| 4321 | tccagtctat taattgttgc cgggaagcta gagtaagtag ttcgccagtt aatagtttgc |
| 4381 | gcaacgttgt tgccattgct acaggcatcg tggtgtcacg ctcgtcgttt ggtatggctt |
| 4441 | cattcagctc cggttcccaa cgatcaaggc gagttacatg atcccccatg ttgtgcaaaa |
| 4501 | aagcggttag ctccttcggt cctccgatcg ttgtcagaag taagttggcc gcagtgttat |
| 4561 | cactcatggt tatggcagca ctgcataatt ctcttactgt catgccatcc gtaagatgct |
| 4621 | tttctgtgac tggtgagtac tcaaccaagt cattctgaga atagtgtatg cggcgaccga |
| 4681 | gttgctcttg cccggcgtca atacgggata ataccgcgcc acatagcaga actttaaaag |
| 4741 | tgctcatcat tggaaaacgt tcttcggggc gaaa |

SEQ ID NO: 11 is the nucleotide sequence of an exemplary plasmid with three lac operator sequences and a heterologous DNA sequence encoding an influenza virus NA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGIGAG CGCAACGCAA TIgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TTGTGAGCGG ATAACAATTa gacccaagct gttaacgcta gcagttaacc |
| 781 | ggagtactgg tcgacctccg aagttggggg ggagcaaaag caggagttta aaATGAATCC |
| 841 | AAATCAAAAG ATAATAACCA TTGGGTCAAT CTGCATGGCA ATTGGAATAA TAAGTCTGGT |
| 901 | GTTACAAATT GGAAATATAA TCTCAATATG GGTTAGTCAT TCAATTCAGA CTGGAAGTCA |
| 961 | GAGCCACCCT GAAACATGCA ATCAAAGTGT CATTACCTAC GAAAACAATA CTTGGGTAAA |
| 1021 | TCAAACATAC GTCAACATAA GTAATACCAA TTTGATTGCA GAACAGACTG TAGCTCCAGT |
| 1081 | AACACTAGCA GGCAATTCCT CTCTCTGTCC CATCAGTGGA TGGGCTATAT ACAGCAAGGA |
| 1141 | CAATGGTATA AGGATAGGTT CTAAGGGAGA TGTATTTGTC ATCAGAGAGC CTTTTATTTC |
| 1201 | ATGCTCTCAC TTGGAATGCA GGACTTTCTT TCTAACTCAA GGGGCCTTGT TGAATGACAA |
| 1261 | GCATTCCAAT GGAACCGTTA AAGACAGAAG CCCCTATAGA ACCCTAATGA GCTGTCCTGT |
| 1321 | TGGTGAAGCT CCCTCTCCAT ACAATTCAAG GITTGAGTCT GTTGCTTGGT CGGCAAGTGC |
| 1381 | TTGCCACGAT GGCATTAGTT GGTTGACAAT TGGTATTTCC GGCCCTGATA ATGGGGCGGT |
| 1441 | GGCTGTATTG AAATACAATG GCATAATAAC AGATACTATC AAGAGTTGGA GAAATAACAT |
| 1501 | ATTGAGAACA CAAGAGTCTG AATGTGCCTG CATTAATGGT TCTTGCTTTA CCATAATGAC |
| 1561 | TGATGGACCA AGTAATGGCC AGGCCTCATA CAAGATTTTC AAGATAGAAA AGGGAAAGGT |
| 1621 | AGTCAAATCA GTTGAGTTGA ATGCCCCTAA TTACCACTAT GAGGAGTGTT CCTGTTATCC |
| 1681 | TGATGCTAGC GAGGTGATGT GTGTATGCAG AGACAACTGG CATGGTTCAA ATCGACCATG |
| 1741 | GGTGTCCTTC GATCAGAATC TAGAGTATCA AATAGGATAC ATATGCAGCG GAGTTTTTGG |
| 1801 | AGACAATCCA CGCCCCAATG ATGGGACAGG CAGTIGTGGT CCAGTGTCTT CTAATGGGGC |
| 1861 | ATATGGGGTA AAAGGGTTTT CATTTAAATA CGGCAACGGT GTTTGGATAG GAAGAACTAA |
| 1921 | AAGTACTAGC TCAAGGAGCG GATTTGAGAT GATTIGGGAT CCCAATGGAT GGACAGAGAC |
| 1981 | GGACAACAGT TTCTCTGTGA AGCAAGACAT TGTAGCAATA ACTGATTGGT CAGGATATAG |
| 2041 | CGGAAGTTTT GTTCAGCATC CAGAGCTGAC AGGACTAGAC TGCATGAGAC CTTGCTTCTG |
| 2101 | GGTTGAGCTA ATCAGGGGAA GACCCAAGGA GAATACAATC TGGACCAGTG GGAGCAGCAT |
| 2161 | TTCCTTTTGT GGAGTAAATA GCGACACTGT GGGTTGGTCT TGGCCAGACG GTGCTGAGTT |
| 2221 | GCCATTCACC ATTGACAAGT AGtttgttca aaaaactcct tgtttctact aataacccgg |
| 2281 | cggcccaaaa tgccgAAATG TGAGCGAGTA ACAACCactc ggagcgaaag atatacctcc |
| 2341 | cccggggccg ggaggtcgcg tcaccgacca cgccgccggc ccaggcgacg cgcgacacgg |
| 2401 | acacctgtcc ccaaaaacgc caccatcgca gccacacacg gagcgcccgg ggccctctgg |
| 2461 | tcaaccccag gacacacgcg ggagcagcgc cgggccgggg acgccctccc ggcggtgacc |
| 2521 | tggccctatt ctatagtgtc acctaaatgc tagagctcgc tgatcagcct cgactgtgcc |
| 2581 | ttctagttgc cagccatctg ttgtttgccc ctcccccgtg ccttccttga ccctggaagg |
| 2641 | tgccactccc actgtccttt cctaataaaa tgaggaaatt gcatcgcatt gtctgagtag |
| 2701 | gtgtcattct attctggggg gtggggtggg gcaggacagc aagggggagg attgggaaga |
| 2761 | caatagcagg catgctgggg atgcggtggg ctctatggct tctgaggcgg aaagaaccag |
| 2821 | ctgcattaat gaatcggcca acgcgcgggg agaggcggtt tgcgtattgg gcgctcttcc |
| 2881 | gcttcctcgc tcactgactc gctgcgctcg gtcgttcggc tgcggcgagc ggtatcagct |
| 2941 | cactcaaagg cggtaatacg gttatccaca gaatcagggg ataacgcagg aaagaacatg |
| 3001 | tgagcaaaag gccagcaaaa ggccaggaac cgtaaaaagg ccgcgttgct ggcgtttttc |
| 3061 | cataggctcc gcccccctga cgagcatcac aaaaatcgac gctcaagtca gaggtggcga |
| 3121 | aacccgacag gactataaag ataccaggcg tttccccctg gaagctccct cgtgcgctct |
| 3181 | cctgttccga ccctgccgct taccggatac ctgtccgcct ttctcccttc gggaagcgtg |
| 3241 | gcgctttctc atagctcacg ctgtaggtat ctcagttcgg tgtaggtcgt tcgctccaag |
| 3301 | ctgggctgtg tgcacgaacc ccccgttcag cccgaccgct gcgccttatc cggtaactat |
| 3361 | cgtcttgagt ccaacccggt aagacacgac ttatcgccac tggcagcagc cactggtaac |
| 3421 | aggattagca gagcgaggta tgtaggcggt gctacagagt tcttgaagtg gtggcctaac |
| 3481 | tacggctaca ctagaagaac agtatttggt atctgcgctc tgctgaagcc agttaccttc |
| 3541 | ggaaaaagag ttggtagctc ttgatccggc aaacaaacca ccgctggtag cggtggtttt |
| 3601 | tttgtttgca agcagcagat tacgcgcaga aaaaaaggat ctcaagaaga tcctttgatc |
| 3661 | ttttctacgg ggtctgacgc tcagtggaac gaaaactcac gttaagggat tttggtcatg |
| 3721 | agattatcaa aaaggatctt cacctagatc cttttaaatt aaaaatgaag ttttaaatca |
| 3781 | atctaaagta tatatgagta aacttggtct gacagttacc aatgcttaat cagtgaggca |
| 3841 | cctatctcag cgatctgtct atttcgttca tccatagttg cctgactccc cgtcgtgtag |
| 3901 | ataactacga tacgggaggg cttaccatct ggccccagtg ctgcaatgat accgcgagac |
| 3961 | ccacgctcac cggctccaga tttatcagca ataaaccagc cagccggaag ggccgagcgc |
| 4021 | agaagtggtc ctgcaacttt atccgcctcc atccagtcta ttaattgttg ccgggaagct |
| 4081 | agagtaagta gttcgccagt taatagtttg cgcaacgttg ttgccattgc tacaggcatc |
| 4141 | gtggtgtcac gctcgtcgtt tggtatggct tcattcagct ccggttccca acgatcaagg |
| 4201 | cgagttacat gatcccccat gttgtgcaaa aaagcggtta gctccttcgg tcctccgatc |
| 4261 | gttgtcagaa gtaagttggc cgcagtgtta tcactcatgg ttatggcagc actgcataat |
| 4321 | tctcttactg tcatgccatc cctaagatgc ttttctgtga ctggtgagta ctcaaccaag |
| 4381 | tcattctgag aatagtgtat gcggcgaccg agttgctctt gcccggcgtc aatacgggat |
| 4441 | aataccgcgc cacatagcag aactttaaaa gtgctcatca ttggaaaacg ttcttcgggg |
| 4501 | cgaaa |

SEQ ID NO: 12 is the nucleotide sequence of an exemplary plasmid with three lac operator sequences, a terminator sequence and a heterologous DNA sequence encoding an influenza virus NA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | aGGCAGTGAG CGCAACGCAA TTgtcatcgc tattaccatg gtgatgcggt tttggcagta |
| 481 | catcaatggg cgtggatagc ggtttgactc acggggattt ccaagtctcc accccattga |
| 541 | cgtcaatggg agtttgtttt ggcaccaaaa tcaacgggac tttccaaaat gtcgtaacaa |
| 601 | ctccgcccca ttgacgcaaa tgggcggtag gcgtgtacgg tgggaggtct atataagcag |
| 661 | agctctctgg ctaactagag aacccactgc ttactggctt atcgaaatta atacgactca |
| 721 | ctatagggAA TTGTGAGCGG ATAACAATTa gacccaagct gttaacgATA AAACGAAAGG |
| 781 | CTCAGTCGAA AGACTGGGCC TITCGTTTTA TCTGTTGTTT GTCGGTGAAC GCTCTCCTGA |
| 841 | GTAGGACAAA TCCGCCGGGA GCGGATTTGA ACGTTGCGAA GCAACGGCCC GGAGGGTGGC |
| 901 | GGGCAGGACG CCCGCCATAA ACTGCCAGGC ATCAAATTAA GCAGAAGGCC ATCCTGACGG |
| 961 | ATGGCCTTTT ctagcagtta accggagtac tggtcgacct ccgaagttgg gggggagcaa |
| 1021 | aagcaggagt ttaaaATGAA TCCAAATCAA AAGATAATAA CCATTGGGTC AATCTGCATG |
| 1081 | GCAATTGGAA TAATAAGTCT GGTGTTACAA ATTGGAAATA TAATCTCAAT ATGGGTTAGT |
| 1141 | CATTCAATTC AGACTGGAAG TCAGAGCCAC CCTGAAACAT GCAATCAAAG TGTCATTACC |
| 1201 | TACGAAAACA ATACTTGGGT AAATCAAACA TACGTCAACA TAAGTAATAC CAATTTGATT |
| 1261 | GCAGAACAGA CTGTAGCTCC AGTAACACTA GCAGGCAATT CCTCTCTCTG TCCCATCAGT |
| 1321 | GGATGGGCTA TATACAGCAA GGACAATGGT ATAAGGATAG GTTCTAAGGG AGATGTATTT |
| 1381 | GTCATCAGAG AGCCTTTTAT TTCATGCTCT CACTTGGAAT GCAGGACTTT CTTTCTAACT |
| 1441 | CAAGGGGCCT TGTTGAATGA CAAGCATTCC AATGGAACCG TTAAAGACAG AAGCCCCTAT |
| 1501 | AGAACCCTAA TGAGCTGTCC TGTTGGTGAA GCTCCCTCTC CATACAATTC AAGGTTTGAG |
| 1561 | TCTGTTGCTT GGTCGGCAAG TGCTTGCCAC GATGGCATTA GTTGGTTGAC AATTGGTATT |
| 1621 | TCCGGCCCTG ATAATGGGGC GGTGGCTGTA TTGAAATACA ATGGCATAAT AACAGATACT |
| 1681 | ATCAAGAGTT GGAGAAATAA CATATTGAGA ACACAAGAGT CTGAATGTGC CTGCATTAAT |
| 1741 | GGTTCTTGCT TTACCATAAT GACTGATGGA CCAAGTAATG GCCAGGCCTC ATACAAGATT |
| 1801 | TTCAAGATAG AAAAGGGAAA GGTAGTCAAA TCAGTTGAGT TGAATGCCCC TAATTACCAC |
| 1861 | TATGAGGAGT GTTCCTGTTA TCCTGATGCT AGCGAGGTGA TGTGTGTATG CAGAGACAAC |
| 1921 | TGGCATGGTT CAAATCGACC ATGGGTGTCC TTCGATCAGA ATCTAGAGTA TCAAATAGGA |
| 1981 | TACATATGCA GCGGAGTTTT TGGAGACAAT CCACGCCCCA ATGATGGGAC AGGCAGTTGT |
| 2041 | GGTCCAGTGT CTTCTAATGG GGCATATGGG GTAAAAGGGT TTTCATTTAA ATACGGCAAC |
| 2101 | GGTGTTTGGA TAGGAAGAAC TAAAAGTACT AGCTCAAGGA GCGGATTTGA GATGATTTGG |
| 2161 | GATCCCAATG GATGGACAGA GACGGACAAC AGTTTCTCTG TGAAGCAAGA CATTGTAGCA |
| 2221 | ATAACTGATT GGTCAGGATA TAGCGGAAGT TTTGTTCAGC ATCCAGAGCT GACAGGACTA |
| 2281 | GACTGCATGA GACCTTGCTT CTGGGTTGAG CTAATCAGGG GAAGACCCAA GGAGAATACA |
| 2341 | ATCTGGACCA GTGGGAGCAG CATTTCCTTT TGTGGAGTAA ATAGCGACAC TGTGGGTTGG |
| 2401 | TCTTGGCCAG ACGGTGCTGA GTTGCCATTC ACCATTGACA AGTAGtttgt tcaaaaaact |
| 2461 | ccttgtttct actaataacc cggcggccca aaatgccgAA ATGTGAGCGA GTAACAACCa |
| 2521 | ctcggagcga aagatatacc tcccccgggg ccgggaggtc gcgtcaccga ccacgccgcc |
| 2581 | ggcccaggcg acgcgcgaca cggacacctg tccccaaaaa cgccaccatc gcagccacac |
| 2641 | acggagcgcc cggggccctc tggtcaaccc caggacacac gcgggagcag cgccgggccg |
| 2701 | gggacgccct cccggcggtg acctggccct attctatagt gtcacctaaa tgctagagct |
| 2761 | cgctgatcag cctcgactgt gccttctagt tgccagccat ctgttgtttg cccctccccc |
| 2821 | gtgccttcct tgaccctgga aggtgccact cccactgtcc tttcctaata aaatgaggaa |
| 2881 | attgcatcgc attgtctgag taggtgtcat tctattctgg ggggtggggt ggggcaggac |
| 2941 | agcaaggggg aggattggga agacaatagc aggcatgctg gggatgcggt gggctctatg |
| 3001 | gcttctgagg cggaaagaac cagctgcatt aatgaatcgg ccaacgcgcg gggagaggcg |
| 3061 | gtttgcgtat tgggcgctct tccgcttcct cgctcactga ctcgctgcgc tcggtcgttc |
| 3121 | ggctgcggcg agcggtatca gctcactcaa aggcggtaat acggttatcc acagaatcag |
| 3181 | gggataacgc aggaaagaac atgtgagcaa aaggccagca aaaggccagg aaccgtaaaa |
| 3241 | aggccgcgtt gctggcgttt ttccataggc tccgcccccc tgacgagcat cacaaaaatc |
| 3301 | gacgctcaag tcagaggtgg cgaaacccga caggactata aagataccag gcgtttcccc |
| 3361 | ctggaagctc cctcgtgcgc tctcctgttc cgaccctgcc gcttaccgga tacctgtccg |
| 3421 | cctttctccc ttcgggaagc gtggcgcttt ctcatagctc acgctgtagg tatctcagtt |
| 3481 | cggtgtaggt cgttcgctcc aagctgggct gtgtgcacga accccccgtt cagcccgacc |
| 3541 | gctgcgcctt atccggtaac tatcgtcttg agtccaaccc ggtaagacac gacttatcgc |
| 3601 | cactggcagc agccactggt aacaggatta gcagagcgag gtatgtaggc ggtgctacag |
| 3661 | agttcttgaa gtggtggcct aactacggct acactagaag aacagtattt ggtatctgcg |
| 3721 | ctctgctgaa gccagttacc ttcggaaaaa gagttggtag ctcttgatcc ggcaaacaaa |
| 3781 | ccaccgctgg tagcggtggt ttttttgttt gcaagcagca gattacgcgc agaaaaaaag |
| 3841 | gatctcaaga agatcctttg atcttttcta cggggtctga cgctcagtgg aacgaaaact |
| 3901 | cacgttaagg gattttggtc atgagattat caaaaaggat cttcacctag atccttttaa |
| 3961 | attaaaaatg aagttttaaa tcaatctaaa gtatatatga gtaaacttgg tctgacagtt |
| 4021 | accaatgctt aatcagtgag gcacctatct cagcgatctg tctatttcgt tcatccatag |
| 4081 | ttgcctgact ccccgtcgtg tagataacta cgatacggga gggcttacca tctggcccca |
| 4141 | gtgctgcaat gataccgcga gacccacgct caccggctcc agatttatca gcaataaacc |
| 4201 | agccagccgg aagggccgag cgcagaagtg gtcctgcaac tttatccgcc tccatccagt |
| 4261 | ctattaattg ttgccgggaa gctagagtaa gtagttcgcc agttaatagt ttgcgcaacg |
| 4321 | ttgttgccat tgctacaggc atcgtggtgt cacgctcgtc gtttggtatg gcttcattca |
| 4381 | gctccggttc ccaacgatca aggcgagtta catgatcccc catgttgtgc aaaaaagcgg |
| 4441 | ttagctcctt cggtcctccg atcgttgtca gaagtaagtt ggccgcagtg ttatcactca |
| 4501 | tggttatggc agcactgcat aattctctta ctgtcatgcc atccgtaaga tgcttttctg |
| 4561 | tgactggtga gtactcaacc aagtcattct gagaatagtg tatgcggcga ccgagttgct |
| 4621 | cttgcccggc gtcaatacgg gataataccg cgccacatag cagaacttta aaagtgctca |
| 4681 | tcattggaaa acgttcttcg gggcgaaa |

SEQ ID NO: 13 is the nucleotide sequence of an exemplary plasmid with two lac operator sequences and a heterologous DNA sequence encoding an influenza virus NA protein.

| | |
|---|---|
| 1 | actctcaagg atcttaccgc tgttgagatc cagttcgatg taacccactc gtgcacccaa |
| 61 | ctgatcttca gcatctttta ctttcaccag cgtttctggg tgagcaaaaa caggaaggca |
| 121 | aaatgccgca aaaaagggaa taagggcgac acggaaatgt tgaatactca tactcttcct |
| 181 | ttttcaatat tattgaagca tttatcaggg ttattgtctc atgagcggat acatatttga |
| 241 | atgtatttag aaaaataaac aaataggggt tccgcgcaca tttccccgaa aagtgccacc |
| 301 | tgacgtcgat atgccaagta cgccccctat tgacgtcaat gacggtaaat ggcccgcctg |
| 361 | gcattatgcc cagtacatga ccttatggga ctttcctact tggcagtaca tctacgtatt |
| 421 | agtcatcgct attaccatgg tgatgcggtt ttggcagtac atcaatgggc gtggatagcg |
| 481 | gtttgactca cggggatttc caagtctcca ccccattgac gtcaatggga gtttgttttg |
| 541 | gcaccaaaat caacgggact ttccaaaatg tcgtaacaac tccgccccat tgacgcaaat |
| 601 | gggcggtagg cgtgtacggt gggaggtcta tataagcaga gctctctggc taactagaga |
| 661 | acccactgct tactggctta tcgaaattaa tacgactcac tatagggAAT TGTGAGCGGA |
| 721 | TAACAATTag acccaagctg ttaacgctag cagttaaccg gagtactggt cgacctccga |
| 781 | agttgggggg gagcaaaagc aggagtttaa aATGAATCCA AATCAAAAGA TAATAACCAT |
| 841 | TGGGTCAATC TGCATGGCAA TTGGAATAAT AAGTCTGGTG TTACAAATTG GAAATATAAT |
| 901 | CTCAATATGG GTTAGTCATT CAATTCAGAC TGGAAGTCAG AGCCACCCTG AAACATGCAA |
| 961 | TCAAAGTGTC ATTACCTACG AAAACAATAC TTGGGTAAAT CAAACATACG TCAACATAAG |
| 1021 | TAATACCAAT TTGATTGCAG AACAGACTGT AGCTCCAGTA ACACTAGCAG GCAATTCCTC |
| 1081 | TCTCTGTCCC ATCAGTGGAT GGGCTATATA CAGCAAGGAC AATGGTATAA GGATAGGTTC |
| 1141 | TAAGGGAGAT GTATTTGTCA TCAGAGAGCC TTTTATTTCA TGCTCTCACT TGGAATGCAG |
| 1201 | GACTTTCTTT CTAACTCAAG GGGCCTTGTT GAATGACAAG CATTCCAATG GAACCGTTAA |
| 1261 | AGACAGAAGC CCCTATAGAA CCCTAATGAG CTGTCCTGTT GGTGAAGCTC CCTCTCCATA |
| 1321 | CAATTCAAGG TTTGAGTCTG TTGCTTGGTC GGCAAGTGCT TGCCACGATG GCATTAGTTG |
| 1381 | GTTGACAATT GGTATTTCCG GCCCTGATAA TGGGGCGGTG GCTGTATTGA AATACAATGG |
| 1441 | CATAATAACA GATACTATCA AGAGTTGGAG AAATAACATA TTGAGAACAC AAGAGTCTGA |
| 1501 | ATGTGCCTGC ATTAATGGTT CTTGCTTTAC CATAATGACT GATGGACCAA GTAATGGCCA |
| 1561 | GGCCTCATAC AAGATTTTCA AGATAGAAAA GGGAAAGGTA GTCAAATCAG TTGAGTTGAA |
| 1621 | TGCCCCTAAT TACCACTATG AGGAGTGTTC CTGTTATCCT GATGCTAGCG AGGTGATGTG |
| 1681 | TGTATGCAGA GACAACTGGC ATGGTTCAAA TCGACCATGG GTGTCCTTCG ATCAGAATCT |
| 1741 | AGAGTATCAA ATAGGATACA TATGCAGCGG AGTTTTTGGA GACAATCCAC GCCCCAATGA |
| 1801 | TGGGACAGGC AGTTGTGGTC CAGTGTCTTC TAATGGGGCA TATGGGGTAA AAGGGTTTTC |
| 1861 | ATTTAAATAC GGCAACGGTG TTTGGATAGG AAGAACTAAA AGTACTAGCT CAAGGAGCGG |
| 1921 | ATTTGAGATG ATTTGGGATC CCAATGGATG GACAGAGACG GACAACAGTT TCTCTGTGAA |
| 1981 | GCAAGACATT GTAGCAATAA CTGATTGGTC AGGATATAGC GGAAGTTTTG TTCAGCATCC |
| 2041 | AGAGCTGACA GGACTAGACT GCATGAGACC TTGCTTCTGG GTTGAGCTAA TCAGGGGAAG |
| 2101 | ACCCAAGGAG AATACAATCT GGACCAGTGG GAGCAGCATT TCCTTTTGTG GAGTAAATAG |
| 2161 | CGACACTGTG GGTTGGTCTT GGCCAGACGG TGCTGAGTTG CCATTCACCA TTGACAAGTA |
| 2221 | Gtttgttcaa aaaactcctt gtttctacta ataacccggc ggcccaaaat gccgAAATGT |
| 2281 | GAGCGAGTAA CAACCactcg gagcgaaaga tatacctccc ccggggccgg gaggtcgcgt |
| 2341 | caccgaccac gccgccggcc caggcgacgc gcgacacgga cacctgtccc caaaaacgcc |
| 2401 | accatcgcag ccacacacgg agcgcccggg gccctctggt caaccccagg acacacgcgg |
| 2461 | gagcagcgcc gggccgggga cgccctcccg gcggtgacct ggccctattc tatagtgtca |
| 2521 | cctaaatgct agagctcgct gatcagcctc gactgtgcct tctagttgcc agccatctgt |
| 2581 | tgtttgcccc tcccccgtgc cttccttgac cctggaaggt gccactccca ctgtcctttc |
| 2641 | ctaataaaat gaggaaattg catcgcattg tctgagtagg tgtcattcta ttctgggggg |
| 2701 | tggggtgggg caggacagca agggggagga ttgggaagac aatagcaggc atgctgggga |
| 2761 | tgcggtgggc tctatggctt ctgaggcgga aagaaccagc tgcattaatg aatcggccaa |
| 2821 | cgcgcgggga gaggcggttt gcgtattggg cgctcttccg cttcctcgct cactgactcg |
| 2881 | ctgcgctcgg tcgttcggct gcggcgagcg gtatcagctc actcaaaggc ggtaatacgg |
| 2941 | ttatccacag aatcagggga taacgcagga aagaacatgt gagcaaaagg ccagcaaaag |
| 3001 | gccaggaacc gtaaaaaggc cgcgttgctg gcgtttttcc ataggctccg cccccctgac |
| 3061 | gagcatcaca aaaatcgacg ctcaagtcag aggtggcgaa acccgacagg actataaaga |
| 3121 | taccaggcgt ttccccctgg aagctccctc gtgcgctctc ctgttccgac cctgccgctt |
| 3181 | accggatacc tgtccgcctt tctcccttcg ggaagcgtgg cgctttctca tagctcacgc |
| 3241 | tgtaggtatc tcagttcggt gtaggtcgtt cgctccaagc tgggctgtgt gcacgaaccc |
| 3301 | cccgttcagc ccgaccgctg cgccttatcc ggtaactatc gtcttgagtc caacccggta |
| 3361 | agacacgact tatcgccact ggcagcagcc actggtaaca ggattagcag agcgaggtat |
| 3421 | gtaggcggtg ctacagagtt cttgaagtgg tggcctaact acggctacac tagaagaaca |
| 3481 | gtatttggta tctgcgctct gctgaagcca gttaccttcg gaaaaagagt tggtagctct |
| 3541 | tgatccggca aacaaaccac cgctggtagc ggtggttttt ttgtttgcaa gcagcagatt |
| 3601 | acgcgcagaa aaaaaggatc tcaagaagat cctttgatct tttctacggg gtctgacgct |
| 3661 | cagtggaacg aaaactcacg ttaagggatt ttggtcatga gattatcaaa aaggatcttc |
| 3721 | acctagatcc ttttaaatta aaaatgaagt tttaaatcaa tctaaagtat atatgagtaa |
| 3781 | acttggtctg acagttacca atgcttaatc agtgaggcac ctatctcagc gatctgtcta |
| 3841 | tttcgttcat ccatagttgc ctgactcccc gtcgtgtaga taactacgat acgggagggc |
| 3901 | ttaccatctg gccccagtgc tgcaatgata ccgcgagacc cacgctcacc ggctccagat |
| 3961 | ttatcagcaa taaaccagcc agccggaagg gccgagcgca gaagtggtcc tgcaacttta |
| 4021 | tccgcctcca tccagtctat taattgttgc cgggaagcta gagtaagtag ttcgccagtt |
| 4081 | aatagtttgc gcaacgttgt tgccattgct acaggcatcg tggtgtcacg ctcgtcgttt |
| 4141 | ggtatggctt cattcagctc cggttcccaa cgatcaaggc gagttacatg atcccccatg |
| 4201 | ttgtgcaaaa aagcggttag ctccttcggt cctccgatcg ttgtcagaag taagttggcc |
| 4261 | gcagtgttat cactcatggt tatggcagca ctgcataatt ctcttactgt catgccatcc |
| 4321 | gtaagatgct tttctgtgac tggtgagtac tcaaccaagt cattctgaga atagtgtatg |
| 4381 | cggcgaccga gttgctcttg cccggcgtca atacgggata ataccgcgcc acatagcaga |
| 4441 | actttaaaag tgctcatcat tggaaaacgt tcttcggggc gaaa |

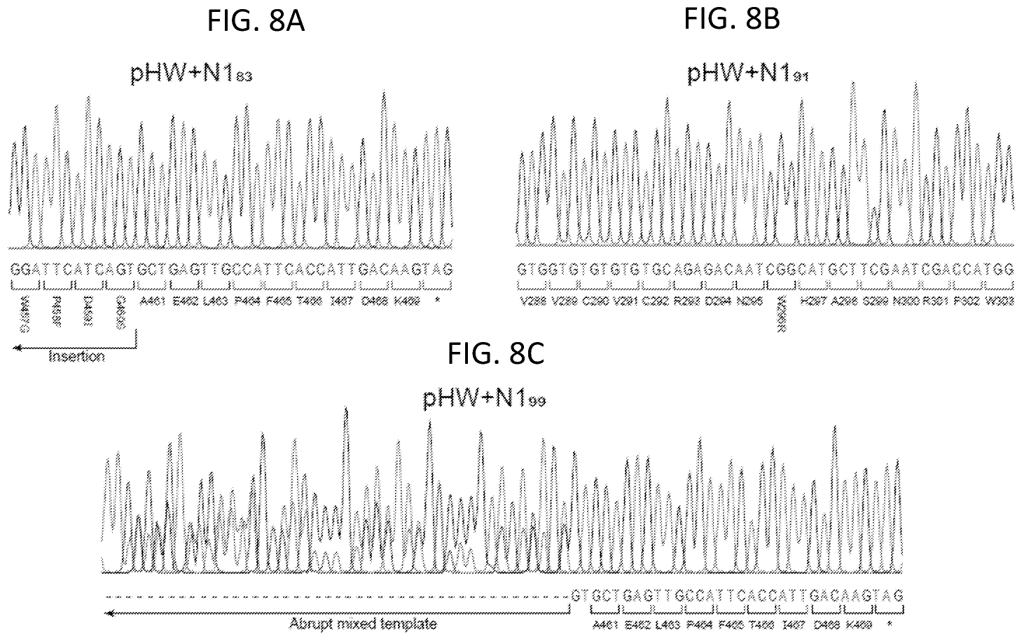

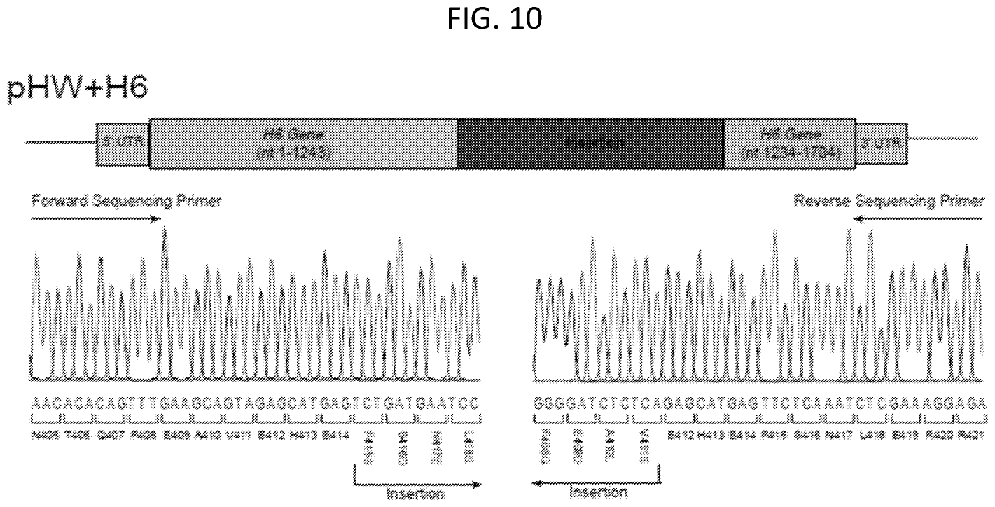

SEQ ID NOs: 14-22 are primer sequences (see Table 1).
SEQ ID NOs: 23-25 are nucleic acid sequences of a region of influenza N1₈₃, N1₉₁ and N1₉₉ genes (see FIGS. 8A-8C).
SEQ ID NOs: 26-27 are nucleic acid sequences of regions of an influenza H6 gene (see FIGS. 10A-10B).

### DETAILED DESCRIPTION

### I. ABBREVIATIONS

CMV cytomegalovirus
EID₅₀ egg infectious dose 50
FSEC fluorescence-detection size exclusion chromatography
HA hemagglutinin
HAU hemagglutination unit
IAV influenza A virus
IPTG isopropyl β-D-1-thiogalactopyranoside
MCS multiple cloning site
NA neuraminidase
NP nucleoprotein
P/S penicillin/streptomycin
RFU relative fluorescent unit
RG reverse genetics
sfGFP superfolder green fluorescent protein
TCID₅₀ tissue culture infectious dose 50
UTR untranslated region
vRNA viral RNA

### II. SUMMARY OF TERMS

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Cloning vector: A nucleic acid molecule or plasmid capable of replicating autonomously in a host cell (e.g., a bacterial cell, such as an E. coli cell). Cloning vectors typically include at least one restriction endonuclease recognition site (e.g., a multiple cloning site) that allows insertion of a heterologous gene, and may also include a selectable marker gene, such as an antibiotic resistance gene.

DNA sequence toxic to E. coli: A heterologous DNA sequence (such as a gene) encoding a protein or transcript that reduces the fitness/growth of E. coli (such as reduces the fitness/growth of E. coli by at least 10%, at least 20%, at least 30%, at least 40%, at least 50%, at least 60%, at least 70%, at least 80%, or at least 90% compared to the fitness/growth of the E. coli in the absence of the heterologous DNA sequence) and/or that is unstable in E. coli (e.g., results in the selection for mutations in the DNA sequence in E. coli). Exemplary DNA sequences toxic to E. coli include, for example, DNA sequences encoding the influenza virus proteins hemagglutinin and neuraminidase. Other microbial DNA sequences toxic to E. coli are known (see, e.g., Kimelman et al., Genome Res 22:802-809, 2012, particularly Supplemental Table S1; Lewin et al., BMC Biotechnol 5:19, 2005; Rose et al., Proc Natl Acad Sci U S A 78:6670-6674, 1981; Gonzalez et al., J Virol 76:4655-4661, 2002; Satyanarayana et al., Virology 313:481-491, 2003; Brosius et al., Gene 27:161-172, 1984).

Escherichia coli (E. coli): A Gram-negative, rod-shaped coliform bacterium that is a facultative anaerobe. Exemplary strains of E. coli include, but are not limited to, XL gold, BL21(DE3), BL21(DE3)pLysS, BL21(DE3)pLysE, DH1, DH41, DH5, DH51, DH51F′, DH51MCR, DH10B, DH10B/p3, DH11S, C600, HB101, JM101, JM105, JM109, JM110, K38, RR1, Y1088, Y1089, CSH18, ER1451 and ER1647.

Expression vector: A nucleic acid molecule or plasmid encoding a gene that can be expressed in a host cell (e.g., a bacterial/prokaryotic cell, such as an E. coli, or a eukaryotic cell, such as mammalian or insect cells). An expression vector can include, for example, a promoter, a heterologous gene (e.g., a gene toxic to E. coli), an origin of replication, a ribosome binding site, a selectable marker gene (such as an antibiotic resistance gene) and/or a gene termination signal (e.g., a poly adenylation sequence).

Hemagglutinin (HA): An influenza virus surface glycoprotein. HA mediates binding of the virus particle to host cells and subsequent entry of the virus into the host cell. HA also causes red blood cells to agglutinate. HA (along with NA) is one of the two major influenza virus antigenic determinants.

Heterologous DNA sequence: In the context of the present disclosure, a “heterologous DNA sequence” refers to a DNA sequence (such as a gene) that is not native to E. coli. In some aspects herein, the heterologous DNA sequence encodes a gene product or a transcript that is toxic to E. coli, such as a viral coding sequence or a transcript to toxic to E. coli when expressed in E. coli.

Influenza virus: A segmented, negative-strand RNA virus that belongs to the Orthomyxoviridae family. Influenza viruses are enveloped viruses. There are three types of influenza viruses, A, B and C.

Influenza A virus (IAV): A negative-sense, single-stranded, segmented RNA virus, which has eight RNA segments (PB2, PB1, PA, NP, M, NS, HA and NA) that code for 10 or more proteins, including RNA-directed RNA polymerase proteins (PB2, PB1 and PA), nucleoprotein (NP), neuraminidase (NA), hemagglutinin (cleaved into subunits HA1 and HA2), the matrix proteins (M1 and M2) and the non-structural proteins (NS1 and NS2). This virus is prone to rapid evolution by error-protein polymerase and by segment reassortment. The host range of influenza A is quite diverse, and includes humans, birds (e.g., chickens and aquatic birds), horses, marine mammals, pigs, bats, mice, ferrets, cats, tigers, leopards, and dogs. Animals infected with influenza A often act as a reservoir for the influenza viruses and certain subtypes have been shown to cross the species barrier to humans.

Influenza A viruses can be classified into subtypes based on allelic variations in antigenic regions of two genes that encode surface glycoproteins, namely, hemagglutinin (HA) and neuraminidase (NA), which are required for viral attachment and mobility. There are currently 18 different influenza A virus HA antigenic subtypes (H1 to H18) and 11 different influenza A virus NA antigenic subtypes (N1 to N11). 1-H16 and N1-N9 are found in wild bird hosts and may be a pandemic threat to humans. H17-H18 and N10-N11 have been described in bat hosts and are not currently thought to be a pandemic threat to humans.

Specific examples of influenza A include, but are not limited to: H1N1 (such as 1918 H1N1), H1N2, H1N7, H2N2 (such as 1957 H2N2), H2N1, H3N1, H3N2, H3N8, H4N8, H5N1, H5N2, H5N8, H5N9, H6N1, H6N2, H6N5, H7N1, H7N2, H7N3, H7N4, H7N7, H7N9, H8N4, H9N2, H10N1, H10N7, H10N8, H11N1, H11N6, H12N5, H13N6, and H14N5. In one example, influenza A includes those known to circulate in humans such as H1N1, H1N2, H3N2, H7N9, and H5N1.

In animals, most influenza A viruses cause self-limited localized infections of the respiratory tract in mammals and/or the intestinal tract in birds. However, highly pathogenic influenza A strains, such as H5N1, cause systemic infections in poultry in which mortality may reach 100%. In 2009, H1N1 influenza was the most common cause of human influenza. A new strain of swine-origin H1N1 emerged in 2009 and was declared pandemic by the World Health Organization. This strain was referred to as “swine flu.” H1N1 influenza A viruses were also responsible for the Spanish flu pandemic in 1918, the Fort Dix outbreak in 1976, and the Russian flu epidemic in 1977-1978.

Influenza B virus (IBV): A negative-sense, single-stranded, RNA virus, which has eight RNA segments. IBV has eight RNA segments (PB1, PB2, PA, HA, NP, NA, M1 and NS1) that code for 10 or more proteins, including RNA-directed RNA polymerase proteins (PB1, PB2 and PA), nucleoprotein (NP), neuraminidase (NA), hemagglutinin (processed into subunits HA1 and HA2), matrix protein (M1), non-structural proteins (NS1 and NS2) and ion channel proteins (NB and BM2). This virus is less prone to evolution than influenza A, but it mutates enough such that lasting immunity has not been achieved. The host range of influenza B is narrower than influenza A as it is only known to infect humans and seals. Influenza B viruses are divided into lineages and strains. Specific examples of influenza B include, but are not limited to: B/Yamagata, B/Victoria, B/Shanghai/361/2002 and B/Hong Kong/330/2001.

Influenza C virus (ICV): A negative-sense, single-stranded, RNA virus, which has seven RNA segments that encode nine proteins. ICV is a genus in the virus family Orthomyxoviridae. ICV infects humans and pigs and generally causes only minor symptoms, but can be severe and cause local epidemics. Unlike IAV and IBV, ICV does not have the HA and NA proteins. Instead, ICV expresses a single glycoprotein called hemagglutinin-esterase fusion (HEF).

Isolated: An “isolated” biological component (such as a nucleic acid, protein, or virus) has been substantially separated or purified away from other biological components (such as cell debris, or other proteins or nucleic acids). Biological components that have been “isolated” include those components purified by standard purification methods. The term also embraces recombinant nucleic acids, proteins, viruses, as well as chemically synthesized nucleic acids or peptides.

Lac operator sequence: A nucleic acid sequence capable of binding an E. coli Lac repressor protein or a variant thereof. In some aspects herein, the lac operator sequence includes or consists of any one of SEQ ID NOs: 1-3. In other aspects, the lac operator sequence includes one or more nucleotide substitutions, deletions or insertions such that the sequence of the lac operator is at least 85% identical to any one of SEQ ID NOs: 1-3, such as at least 90%, at least 95%, at least 96%, at least 97%, at least 98% or at least 99% identical to any one of SEQ ID NOs: 1-3, while retaining the ability to bind an E. coli Lac repressor protein having an amino acid sequence at least 85% identical to SEQ ID NO: 5, such as at least 90%, at least 95%, at least 96%, at least 97%, at least 98% or at least 99% identical to SEQ ID NO: 5. lac operator sequence variants are known, such as those described in Du et al., Nucleic Acids Res 47(18):9609-9618, 2019; Maity et al., FEBS J 279:2534-2543, 2012; and Garcia et al., Cell Reports 2:150-161, 2012. In some examples, the nucleotide substitution(s), deletion(s) or insertion(s) is/are located in an internal region of the operator sequence (such as at least 3, at least 4, at least 5, at least 6 or at least 7 nucleotides from either terminus).

Lac repressor: A dimeric protein expressed by bacteria such as E. coli that can bind to one lac operator sequence of the E. coli lac operon. Interactions between bound Lac repressor dimers can also result in the formation of tetramers that can spatially link any two lac operators. In some aspects, the amino acid sequence of the Lac repressor protein includes or consists of SEQ ID NO: 5. In other aspects, the Lac repressor protein includes one or more amino acid substitutions, deletions or insertions such that the amino acid sequence of the Lac repressor protein is at least 85%, at least 90%, at least 95%, at least 96%, at least 97%, at least 98% or at least 99% identical to SEQ ID NO: 5, while retaining the ability to bind one or more lac operator sequences. In some examples, the modified Lac repressor protein includes modifications to the DNA binding site and/or the lactose binding site (see residues in bold underline in SEQ ID NO: 5, which form the substrate binding pocket). Modified Lac repressor sequences are known, such as those described in Kwon et al., Sci Rep 5:16076, 2015; Pfahl, J Bacteriol 137(1):137-145; and Gatti-Lafranconi et al., Microb Cell Fact 12:67).

Multiple cloning site (MCS): A region of DNA that includes recognition sequences for more than one restriction endonuclease. An MCS is typically no more than 200, no more than 150, no more than 100 or no more than 50 nucleotides in length and includes at least 5, at least 6, at least 7, at least 8, at least 9, at least 10, at least 11, at least 12, at least 13, at least 14, at least 15, at least 16, at least 17, at least 18, at least 19 or at least 20 restriction sites.

Neuraminidase (NA): An influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Origin of replication (ori): A specific DNA sequence in a genome or plasmid where DNA replication is initiated.

Plasmid: A circular DNA capable of replicating independently of host cell chromosomes. To replicate, a plasmid includes an origin of replication. Plasmids can be used, for example, for cloning and/or expressing a gene of interest.

Promoter: An array of nucleic acid control sequences that directs transcription of a nucleic acid. A promoter includes necessary nucleic acid sequences near the start site of transcription, such as in the case of a polymerase II type promoter (a TATA element). A promoter also optionally includes distal enhancer or repressor elements that can be located as much as several thousand base pairs from the start site of transcription. Both constitutive and inducible promoters are included. In some aspects herein, the promoter is a cytomegalovirus (CMV) promoter, an RNA polymerase I promoter, or an RNA polymerase II promoter.

Ribosome binding site: A nucleic acid sequence located upstream of a start codon of a mRNA transcript that enables recruitment of a ribosome for translation of the transcript.

Selectable marker: A nucleic acid sequence (such as a gene) encoding a protein that confers the ability of a cell (such as a bacterial cell) to grow in the presence of a selective agent. For example, the selectable marker can be an antibiotic resistance gene that enables the cell to grow in the presence of the corresponding antibiotic.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genet. 6:119-129, 1994.

The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Terminator sequence: A nucleic acid sequence that mediates termination of transcription. In some aspects herein, the terminator sequence is derived from the transcription termination region of the rrnB gene of E. coli. In specific examples, the terminator sequence includes or consists of the nucleotide sequence of SEQ ID NO: 4. In other examples, the terminator sequence is at least 85%, at least 90%, at least 95%, at least 96%, at least 97%, at least 98% or at least 99% identical to SEQ ID NO: 4.

Under conditions sufficient to: A phrase that is used to describe any environment that permits the desired activity.

### III. RECOMBINANT NUCLEIC ACID MOLECULES AND PLASMIDS FOR EXPRESSION OF TOXIC HETEROLOGOUS DNA IN E. COLI

The present disclosure describes recombinant nucleic acid molecules engineered for efficient propagation of a heterologous DNA sequence (such as a heterologous viral gene) that reduces the fitness and/or growth of E. coli and/or that is unstable in E. coli (e.g., toxic). The toxic DNA sequence (e.g., a gene) can encode, for example, a protein or transcript that is directly toxic to E. coli (e.g., impairs fitness, growth, or induces cell death) resulting in the selection for mutations in the DNA sequence that decrease the toxicity in E. coli. It is disclosed herein that exemplary toxic heterologous DNA sequences cloned into plasmids can be transcribed and translated in E. coli and that the toxicity of the heterologous DNA is mitigated by introducing regulatory elements that decrease gene transcription in E. coli.

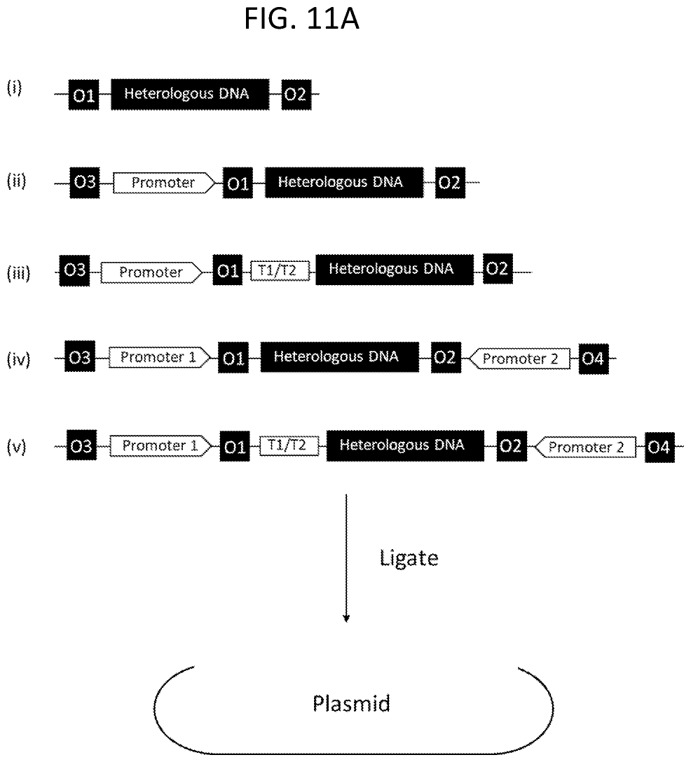

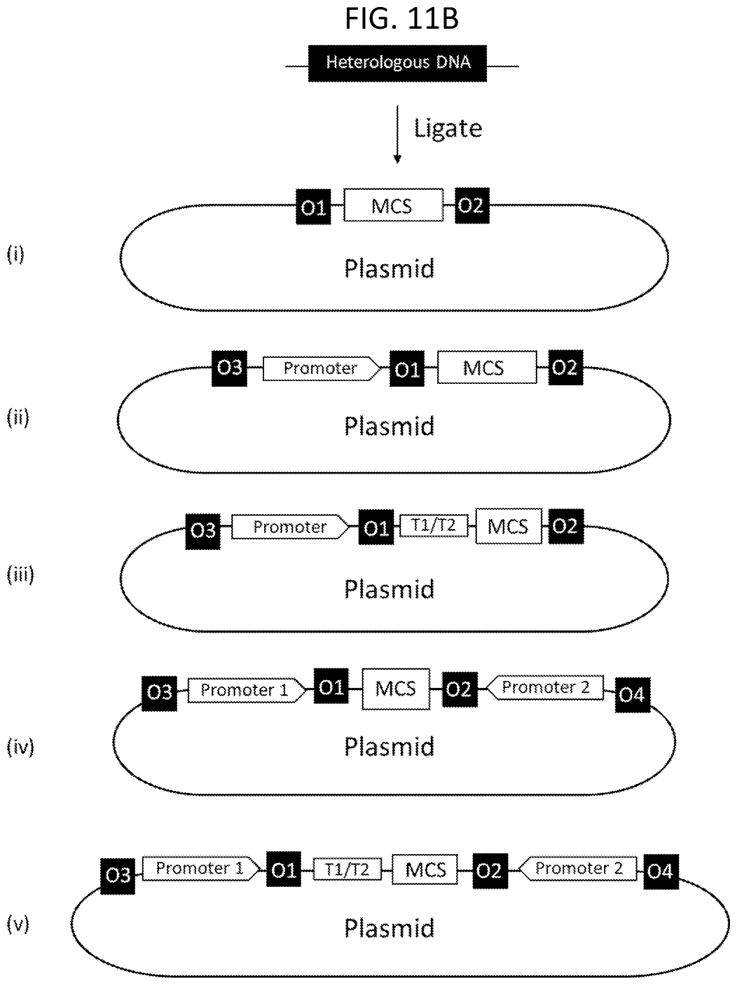

Provided herein are recombinant nucleic acid molecules that include, in the 5′ to 3′ direction, a first lac operator sequence, a heterologous DNA sequence, and a second lac operator sequence (see FIG. 11A for non-limiting examples). Also provided herein are recombinant nucleic acid molecules that include, in the 5′ to 3′ direction, a first lac operator sequence, a multiple cloning site (MCS) for insertion of a heterologous DNA sequence, and a second lac operator sequence (see FIG. 11B for non-limiting examples). The heterologous DNA sequence can, for example, encode a protein or transcript that is toxic to E. coli, or that is unstable in E. coli.

In some aspects, the recombinant nucleic acid molecule includes first and second lac operator sequences that flank the heterologous DNA sequence (such as at positions O1 and O2 in FIG. 11A(i)), or that flank the MCS (such as at positions O1 and O2 in FIG. 11B(i)), but does not include any additional operator sequences, a terminator sequence, or a promoter. In other aspects, the recombinant nucleic acid molecule further includes at least one promoter (such as one promoter, two promoters, three promoters or four promoters), a third lac operator sequence and/or a fourth lac operator sequence (such as at positions O3 and O4, respectively, in FIGS. 11A-11B).

In FIGS. 11A-11B, O1, O2, O3 and O4 represent first, second, third and fourth (respectively) positions where operator sequences are present, but do not represent specific nucleic acid sequences (e.g., the operator sequence at position O1 can have the same sequence as the operator sequence at position O2, or the sequences can be different). In some examples, the first and second lac operator sequences are the same sequence (for example, both operator sequences have the nucleotide sequence of SEQ ID NO: 1). In other examples, the first and second lac operator sequences are different lac operator sequence (for example, the first operator sequence includes SEQ ID NO: 1 and the second operator sequence includes SEQ ID NO: 2). Similarly, in some examples, the first and second lac operator sequences, and the optional third and fourth lac operator sequences, are all the same sequence. In other examples, first and second lac operator sequences and the optional third and fourth lac operator sequences, are all different lac operator sequences. In yet other examples, at least two or at least three of the first, second, third and fourth lac operator sequences are different sequences. In yet other examples, at least two or at least three of the first, second, third and fourth lac operator sequences are identical sequences.

In some examples, the recombinant nucleic acid molecule includes a first promoter located 5′ of the first lac operator sequence or located 3′ of the second lac operator sequence, or includes a first promoter located 5′ of the first lac operator sequence and a second promoter located 3′ of the second lac operator sequence (such as a promoter in the reverse orientation). In particular examples, the recombinant nucleic acid molecule includes first and second lac operator sequences that flank the heterologous DNA sequence or the MCS, a promoter 5′ of the first lac operator sequence and optionally a third lac operator sequence located 5′ of the first promoter (see FIG. 11A(ii) and FIG. 11B(ii)). In other particular examples, the recombinant nucleic acid molecule includes, in the 5′ to 3′ direction, an optional third lac operator sequence, a first promoter, a first lac operator sequence, the heterologous DNA sequence or MCS, a second lac operator sequence, a second promoter, and an optional fourth lac operator sequence (see FIG. 11A(iv) and FIG. 11B(iv)).

In other aspects, the recombinant nucleic acid molecule includes or further includes a terminator sequence. In some examples, a terminator sequence is at least 50 nucleotides (nt), at least 100 nt, at least 200 nt, at least 300 nt, at least 400 nt, or at least 500 nt, such as 50-1000 nt, 100-500 nt, or 150-300 nt, such as 50, 75, 100, 125, 150, 200, 225, 250, 275, 300, 350, 400, 450, 500, 550, or 1000 nt. In specific examples, the terminator sequence includes or consists of the nucleotide sequence of SEQ ID NO: 4. In other examples, the terminator sequence is at least 85%, at least 90%, at least 95%, at least 96%, at least 97%, at least 98% or at least 99% identical to SEQ ID NO: 4.

In some examples, the recombinant nucleic acid molecule includes a first lac operator sequence 5′ of the heterologous DNA sequence or MCS, a second lac operator sequence 3′ of the heterologous DNA sequence or MCS, and a terminator sequence positioned between the first lac operator sequence and the heterologous DNA sequence or MCS. In particular examples, the recombinant nucleic acid molecule includes, in the 5′ to 3′ direction, an optional third lac operator sequence, a promoter, a first lac operator sequence, a terminator sequence, a heterologous DNA sequence or MCS, and a second lac operator sequence (see FIG. 11A(iii) and FIG. 11B(iii)). In other particular examples, the recombinant nucleic acid molecule includes, in the 5′ to 3′ direction, an optional third lac operator sequence, a first promoter, a first lac operator sequence, a terminator sequence, a heterologous DNA sequence or MCS, a second lac operator sequence, a second promoter, and an optional fourth lac operator sequence (see FIG. 11A(v) and FIG. 11B(v)).

In some aspects, the recombinant nucleic acid molecule further includes a sequence encoding an E. coli Lac repressor protein having the amino acid sequence of SEQ ID NO: 5, or a variant thereof having at least 85%, at least 90%, at least 91%, at least 92%, at least 93%, at least 94%, at least 95%, at least 96%, at least 97%, at least 98%, or at least 99% identical to SEQ ID NO: 5. In some examples, the amino acid sequence of the Lac repressor protein consists of SEQ ID NO: 5. The sequence encoding an E. coli Lac repressor protein can be located in any position that does not overlap with the heterologous DNA, lac operator sequences, terminator sequence or promoter(s). In some examples, the recombinant nucleic acid molecule further includes a promoter (such as a bacterial promoter) upstream of the sequence encoding the E. coli Lac repressor protein to drive expression of the repressor.

### A. Exemplary Promoters

In the context of the recombinant nucleic acid molecules disclosed herein, the promoter, the first promoter and/or the second promoter can be a bacterial promoter (such as, but not limited to, an E. coli RNA polymerase promoter, a T7 promoter or a T4 promoter) or a mammalian promoter (such as, but not limited to, an RNA polymerase I promoter, RNA polymerase II promoter or RNA polymerase III promoter). In some aspects, the first promoter is a mammalian promoter and the second promoter is a bacterial promoter. In other aspects, the first promoter is a bacterial promoter and the second promoter is a mammalian promoter. In other aspects, the first promoter and the second promoter are both mammalian promoters (either the same mammalian promoter, or two different mammalian promoters). In yet other aspects, the first promoter and the second promoter are both bacterial promoters (either the same bacterial promoter, or two different bacterial promoters).

### B. Exemplary Lac Operators

The lac operator sequences of the disclosed recombinant nucleic acid molecules can be wild-type lac operator sequences, or can be variants of a lac operator sequence that retain the capacity to bind the Escherichia coli Lac repressor protein of SEQ ID NO: 5, or a variant of the Lac repressor protein having at least 90%, at least 91%, at least 92%, at least 93%, at least 94%, at least 95%, at least 96%, at least 97%, at least 98%, at least 99% sequence identity to SEQ ID NO: 5. In some aspects, the first lac operator sequence, the second lac operator sequence, the optional third lac operator sequence and/or the optional fourth lac operator sequence are individually selected from SEQ ID NO: 1, SEQ ID NO: 2, SEQ ID NO: 3, a sequence at least 85%, at least 90%, at least 91%, at least 92%, at least 93%, at least 94%, at least 95%, at least 96%, at least 97%, at least 98%, or at least 99% identical to SEQ ID NO: 1, a sequence at least 85%, at least 90%, at least 91%, at least 92%, at least 93%, at least 94%, at least 95%, at least 96%, at least 97%, at least 98%, or at least 99% identical to SEQ ID NO: 2, and a sequence at least 85%, at least 90%, at least 91%, at least 92%, at least 93%, at least 94%, at least 95%, at least 96%, at least 97%, at least 98%, or at least 99% identical to SEQ ID NO: 3. In particular examples, the recombinant nucleic acid molecule includes a first operator sequence of SEQ ID NO: 1, a second lac operator sequence of SEQ ID NO: 2 and a third lac operator sequence of SEQ ID NO: 3. In some examples, a lac operator is at least 15 nucleotides (nt), at least 20 nt, or at least 25 nt, such as 15-30 nt, 15-25 nt, or 20-25 nt, such as 15, 16, 17, 81, 19, 20, 21, 22, 23, 24, 25, 26, 27, 28, 29, 30, 31, 32, 33, 34, or 35 nt.

### C. Exemplary Heterologous DNA Sequences

In some aspects of the disclosed recombinant nucleic acid molecules, the heterologous DNA sequence encodes a protein or transcript that is toxic to E. coli. In some examples, the heterologous DNA sequence encodes a protein or transcript from a virus, such as a DNA virus, RNA virus, or retrovirus. In one example, the heterologous DNA sequence encodes a protein or transcript from a retrovirus, such as Rous sarcoma virus, HIV-1, HIV-2, and feline leukemia virus. In one example, the heterologous DNA sequence encodes a protein or transcript from a DNA virus, such as a double-or single-stranded DNA virus, hepatitis B virus, a Cytomegalovirus (CMV), herpesviruses, papillomaviruses, and poxviruses. In one example, the heterologous DNA sequence encodes a protein or transcript from an RNA virus, such as a single-stranded RNA virus (such as a positive or negative ssRNA virus) or double-stranded RNA virus. In one example, the heterologous DNA sequence encodes a protein or transcript from an RNA virus, such as a protein or transcript from influenza, SARS, MERS, SARS-CoV-2 (or any variant thereof), a Flavivirus (such as West Nile virus, a dengue virus, yellow fever virus, Zika virus, hepatitis C virus, and Kunjin virus), hepatitis E virus, Ebola virus, rabies virus, poliovirus, mumps virus, and measles virus. In some examples, the protein or transcript encoded by a virus is one from any one of the following virus families: Orthomyxoviridae (for example, influenza viruses, such as human influenza A virus (IAV), IBV, ICV); Paramyxoviridae (for example, parainfluenza viruses, mumps virus, measles virus, respiratory syncytial virus); Retroviridae (for example, human immunodeficiency virus (HIV), human T-cell leukemia viruses); Picornaviridae (for example, poliovirus, hepatitis A virus, enteroviruses, human coxsackie viruses, rhinoviruses, echoviruses, foot-and-mouth disease virus); Caliciviridae (such as Norwalk virus); Togaviridae (for example, alphaviruses (including chikungunya virus, equine encephalitis viruses, Simliki Forest virus, Sindbis virus, Ross River virus, rubella viruses)); Flaviridae (for example, hepatitis C virus, dengue viruses, yellow fever viruses, West Nile virus, St. Louis encephalitis virus, Japanese encephalitis virus, Powassan virus and other encephalitis viruses); Coronaviridae (for example, coronaviruses, severe acute respiratory syndrome coronavirus (SARS-CoV) and SARS-CoV-2, Middle East respiratory syndrome (MERS) virus); Rhabdoviridae (for example, vesicular stomatitis viruses, rabies viruses); Filoviridae (for example, Ebola virus, Marburg virus); Bunyaviridae (for example, Hantaan viruses, Sin Nombre virus, Rift Valley fever virus, bunya viruses, phleboviruses and Nairo viruses); Arenaviridae (such as Lassa fever virus and other hemorrhagic fever viruses, Machupo virus, Junin virus); Reoviridae (e.g., reoviruses, orbiviruses, rotaviruses); Birnaviridae; Hepadnaviridae (such as hepatitis B virus); Parvoviridae (for example, parvoviruses); Papovaviridae (for example, papilloma viruses, polyoma viruses, BK-virus); Adenoviridae (such as human adenoviruses of any one of 88 serotypes); Herpesviridae (e.g., herpes simplex virus (HSV)-1 and HSV-2; cytomegalovirus; Epstein-Barr virus; varicella zoster virus; Kaposi's sarcoma herpesvirus (KSHV); other herpes viruses, including HSV-6); Poxviridae (for example, variola viruses, vaccinia viruses, pox viruses); Iridoviridae (such as African swine fever virus); and Astroviridae.

In some examples, the heterologous DNA sequence encodes a protein or transcript from an influenza virus, such as an influenza A virus (IAV), for example H1N1 (such as 1918 H1N1), H1N2, H1N7, H2N2 (such as 1957 H2N2), H2N1, H3N1, H3N2, H3N8, H4N8, H5N1, H5N2, H5N8, H5N9, H6N1, H6N2, H6N5, H7N1, H7N2, H7N3, H7N4, H7N7, H7N9, H8N4, H9N2, H1ON1, H10N7, H10N8, H11N1, H11N6, H12N5, H13N6, or H14N5. In specific examples, the influenza virus protein or transcript is an influenza virus hemagglutinin (HA) protein or transcript, or an influenza virus neuraminidase (NA) protein or transcript. In particular non-limiting examples, the heterologous DNA sequence includes or consists of nucleotides 809-2512 of SEQ ID NO: 10 (an exemplary HA gene) or includes or consists of nucleotides 812-2221 of SEQ ID NO: 13 (an exemplary NA gene).

In some examples, the heterologous DNA sequence encodes a protein or transcript from a SARS-CoV-2 virus, or variant thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (B.1.1.529 and lineages thereof such as BA.1, BA.2, BA3, BA.4, and BA.5). In specific examples, the SARS-CoV2 virus protein or transcript is a SARS-CoV-2 virus spike protein or transcript, such as an S1 subunit or S2 subunit protein or transcript.

In other examples, the heterologous DNA sequence encodes a protein or transcript from a non-viral microbe, such as a bacterium, parasite, or fungus. Exemplary heterologous DNA sequences (such as genes) toxic to E. coli are known (see, e.g., Kimelman et al., Genome Res 22:802-809, 2012, particularly Supplemental Table S1 [Supplemental Table S1 herein incorporated by reference in its entirety]; Lewin et al., BMC Biotechnol 5:19, 2005; Rose et al., Proc Natl Acad Sci U S A 78:6670-6674, 1981; Gonzalez et al., J Virol 76:4655-4661, 2002; Satyanarayana et al., Virology 313:481-491, 2003; Brosius et al., Gene 27:161-172, 1984).

### D. Exemplary Plasmids

Also provided herein are plasmids, such as expression plasmids or cloning plasmids, that include a recombinant nucleic acid molecule disclosed herein. In some aspects of the disclosed plasmids, the heterologous DNA sequence is a viral gene, such as a gene from an RNA virus, DNA virus, or retrovirus (specific examples provided above). In a specific example, the heterologous DNA sequence is a gene encoding an influenza virus HA or NA protein.

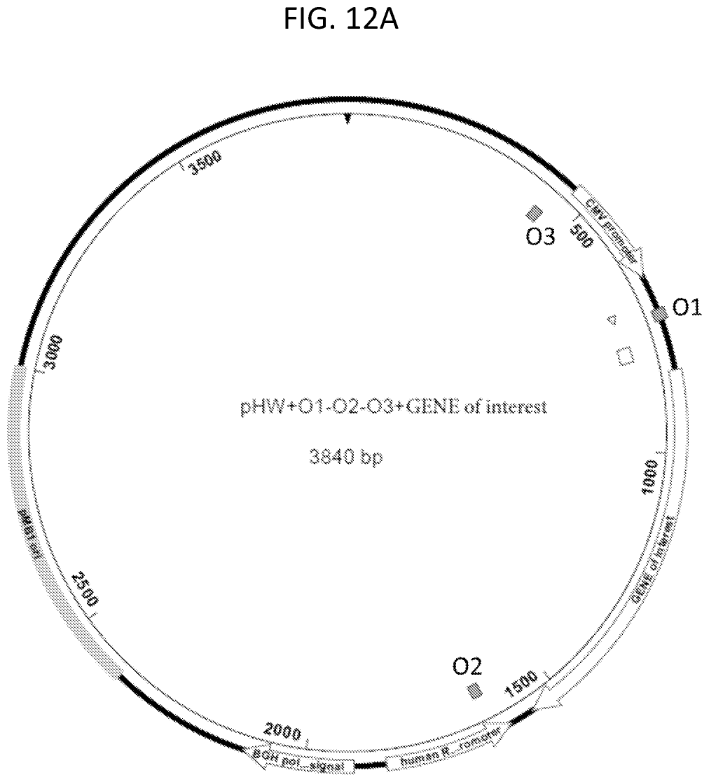

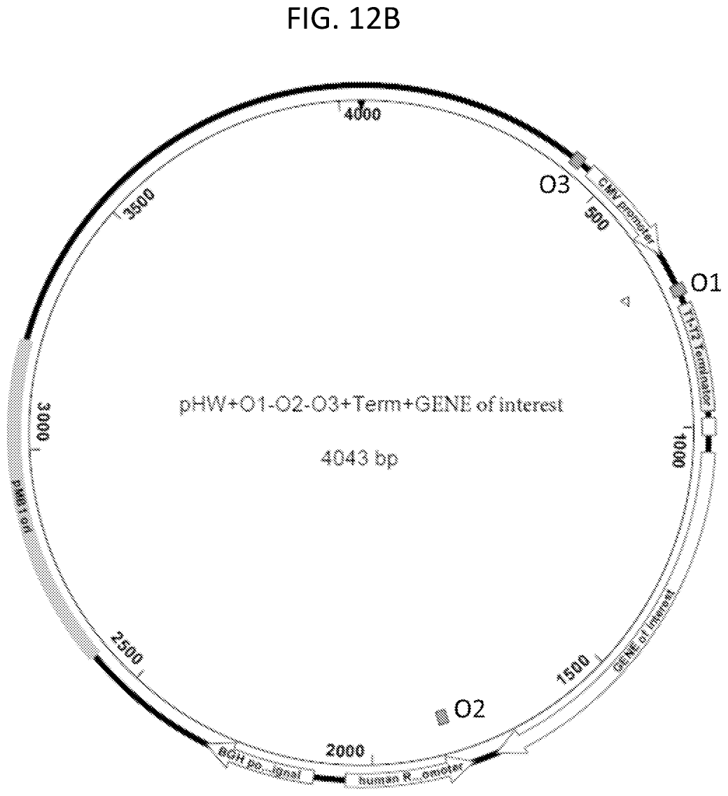

In some aspects, the plasmid further includes an origin of replication, a selectable marker gene, a ribosome binding site, a gene termination signal, or any combination thereof (see, e.g., FIGS. 12A-12B).

In some examples, the nucleotide sequence of the plasmid is at least 85%, at least 90%, at least 91%, at least 92%, at least 93%, at least 94%, at least 95%, at least 96%, at least 97%, at least 98%, or at least 99% identical to SEQ ID NO: 8, SEQ ID NO: 9, SEQ ID NO: 10, SEQ ID NO: 11, SEQ ID NO: 12 or SEQ ID NO: 13. In specific non-limiting examples, the nucleotide sequence of the plasmid includes or consists of SEQ ID NO: 8, SEQ ID NO: 9, SEQ ID NO: 10, SEQ ID NO: 11, SEQ ID NO: 12 or SEQ ID NO: 13.

In particular examples, provided is a plasmid that includes, in the 5′ to 3′ direction, a lac operator sequence that includes the nucleotide sequence of SEQ ID NO: 3; a promoter; a lac operator sequence that includes the nucleotide sequence of SEQ ID NO: 1; an influenza virus HA or NA gene; and a lac operator sequence that includes the nucleotide sequence of SEQ ID NO: 2.

In other particular examples, provided is a plasmid that includes, in the 5′ to 3′ direction, a lac operator sequence that includes the nucleotide sequence of SEQ ID NO: 3; a promoter; a lac operator sequence that includes the nucleotide sequence of SEQ ID NO: 1; a terminator sequence that includes the nucleotide sequence of SEQ ID NO: 4; an influenza virus hemagglutinin or neuraminidase gene; and a lac operator sequence that includes the nucleotide sequence of SEQ ID NO: 2.

Further provided herein are methods of propagating a plasmid in E. coli, wherein the plasmid includes a heterologous DNA sequence that is toxic to E. coli. In some aspects, the method includes transforming E. coli with a plasmid (such as a cloning plasmid or expression plasmid) disclosed herein under conditions sufficient to allow replication of the plasmid, thereby propagating the plasmid in E. coli. In some aspect, the heterologous DNA sequence toxic to E. coli is an influenza virus gene, such as an HA or NA gene.

### E. Exemplary Kits

Kits that include a recombinant nucleic acid molecule or a plasmid disclosed herein are also provided. The kits can further include, for example, one or more restriction endonucleases, buffer, culture media (such as a solid or liquid culture media), one or more antibiotics, one or more ligases, primers, reverse transcriptase, deoxyribonucleotide triphosphates (dNTPs), one or more reagents to induce a promoter, cells (such as prokaryotic cells or eukaryotic cells), or a combination thereof. In some examples, the kit includes a ligase. In some examples, the kit includes one or more reagents to activate a promoter, such as IPTG. In some examples, the kit includes cells, such as E. coli cells, which may be in a liquid or solid media, or may be frozen. In some examples, components of a kit are present in separate vials or containers, which in some examples are composed of glass, metal, or plastic.

### F. Exemplary Cells

Also provided are isolated cells that include a recombinant nucleic acid molecule or plasmid disclosed herein. In the isolated cells, the recombinant nucleic acid molecule or plasmid is in a complex with an E. coli Lac repressor protein or a variant thereof. In some aspects, the Lac repressor protein or variant thereof has an amino acid sequence at least 85%, at least 90%, at least 91%, at least 92%, at least 93%, at least 94%, at least 95%, at least 96%, at least 97%, at least 98%, or at least 99% sequence identity to SEQ ID NO: 5. In some examples, the amino acid sequence of the Lac repressor protein includes or consists of the amino acid sequence of SEQ ID NO: 5. In some examples, the isolated cell is an E. coli cell.

The following examples are provided to illustrate certain particular features and/or aspects. These examples should not be construed to limit the disclosure to the particular features or aspects described.

### EXAMPLES

The following Examples describe studies to overcome difficulties in cloning specific neuraminidase (NA) and hemagglutinin (HA) gene segments into a common plasmid for IAV reverse genetics. The disclosed studies examined if the influenza gene segment or the reverse genetics plasmid was responsible for the instability in E. coli. The results using a reporter gene (sfgfp) demonstrated that genes cloned into the reverse genetics plasmid could be transcribed and translated in E. coli and that the toxicity of the influenza gene segments was mitigated by introducing regulatory elements that decrease sfgfp transcription/translation in E. coli. The largest stability increase for influenza virus genes was observed from a plasmid where the viral genes were situated between lac operators, and it was demonstrated that IAVs can be efficiently rescued using this modified reverse genetics plasmid. Based on this data, a skilled person will appreciate that such methods can be used for other toxic genes, such as those encoded by a DNA or RNA virus.

### Example 1: Materials and Methods

This example describes the materials and experimental procedures for the studies described in Examples 2-9.

### Reagents

Dulbecco's Modified Eagles Medium (DMEM), fetal bovine serum (FBS), L-glutamine, penicillin/streptomycin (P/S), Opti-MEM I (OMEM), Simple Blue Stain, Novex 4-12% Tris-Glycine SDS-PAGE gels, Novex Sharp Unstained Protein Standard, GeneRuler 1kb Plus DNA Ladder, LB Medium Dehydrated Capsules, and the Phusion High-Fidelity DNA Polymerase were all purchased from Thermo Fisher Scientific. His-tagged Pfu X7 DNA Polymerase was prepared in-house by Immobilized Metal Affinity Chromatography (IMAC) for routine PCR-based bacterial colony screening. XL10-Gold Ultracompetent cells, which are lacI^{q}, were acquired from Agilent Technologies, Inc. SIGMAFAST EDTA-free Protease Inhibitor cocktail tablets, DpnI, TransIT-LT1 transfection reagent, and 2′-(4-methylumbelliferyl)-α-d-N-acetylneuraminic acid (MUNANA) were obtained from Sigma-Aldrich, New England Biolabs, Mirus Bio, and Cayman Chemicals, respectively. Specific-Pathogen-Free (SPF) eggs and turkey red blood cells (TRBCs) were purchased from Charles River Labs and the Poultry Diagnostic and Research Center (Athens, GA), respectively. All primers (Table 1) were synthesized by Integrated DNA Technologies.

TABLE 1
Primers for cloning the NA and HA gene segments in the various pHW plasmids

| Plasmid | |
|---|---|
| backbone/Insert | Primers |
| *pHW | FWD: 5′-GTTTCTACTaataacccggcggcccaaaatg-3′ (SEQ ID NO: 14) |
| | REV: 5′-CCTGCTTTTGCTcccccccaacttcggaggtcgaccagtac-3′ (SEQ ID NO: 15) |
| NA | FWD: 5′-ctggtcgacctccgaagttgggggggAGCAAAAGCAGGAGTTTAAAATG-3′ (SEQ ID NO: 16) |
| | REV: 5′-gcattttgggccgccgggttattAGTAGAAACAAGGAGTTTTTTGAAC-3′ (SEQ ID NO: 17) |
| H1 | FWD: 5′-ggtcgacctccgaagttgggggggAGCAAAAGCAGGGGAAAACAAAAGC-3′ (SEQ ID NO: 18) |
| | REV: 5′-gcattttgggccgccgggttattAGTAGAAACAAGGGTGTTTTTCTC-3′ (SEQ ID NO: 19) |
| H6 | FWD: 5′-ctggtcgacctccgaagttgggggggAGCAAAAGCAGGGGAAAATG-3′ (SEQ ID NO: 20) |
| | REV: 5′-gcattttgggccgccgggttattAGTAGAAACAAGGGTGTTTTTTTCTAATTATATAC-3′ (SEQ |
| | ID NO: 21) |
| pHW Screen | FWD: 5′-agcagttaaccggagtactggtcg-3′ (SEQ ID NO: 22) |

Primers used for the simplified Gibson assembly method and colony screening are shown. Overlapping regions complementary to the termini of the amplified pHW backbone are indicated with single (3′ end of insert) and double (5′ end of insert) underlines. Lower case nucleotides correspond to vector sequence and upper case denote the influenza gene specific sequence. For colony screening, pHW screen was paired with the NA, H1 or H6 reverse (Rev) primer. * All pHW variant plasmids were amplified with these primers.

### Plasmids and Constructs

The eight WSN (A/WSN/33) and PR8 (A/PR/8/34) reverse genetics (RG) plasmids have been previously described (Hoffmann et al., Proc Natl Acad Sci USA 97(11):6108-6113, 2000). The RG plasmids were sequenced and correspond with the following GenBank Identifications: LC333182.1 (WSN33-PB2), LC333183.1 (WSN33-PB1), LC333184.1 (WSN33-PA), LC333185.1 (WSN33-HA), LC333186.1 (WSN33-NP), MF039638.1 (WSN33-M) LC333189.1 (WSN33-NS). Generation of the NA (N1-BR18; GISAID ID: EPI1212833) RG plasmid has been described previously (Gao et al., PLoS Pathog 17(4):e1009171, 2021). To create the NA (Human H1N1 (1935-2019), Avian H1N1 (1976-2019)) and HA (H1-BR18 (GISAID ID: EPI1212834) and H6 (GenBank ID: CY087752.1)) RG plasmids, the NA and HA gene segments with their respective 5′ and 3′ untranslated regions (UTRs) were amplified by PCR from commercially synthesized gene segments in pUC57 (GenScript USA). The amplified constructs were then cloned into a PCR amplified pHW2000 (referred to herein as pHW) plasmid backbone (Hoffman Webster PNAS 2000) using a simplified Gibson assembly method, which involves mixing the Dpnl treated PCR reactions at 3:1 molar ratio of insert: vector prior to transformation (Mellroth et al., J Biol Chem 287(14):11018-11029, 2012). The superfolder GFP (sfGFP) gene was synthesized together with different combinations of the lac operators (pHW-sfGFP, pHWO₁₂₃-sfGFP (FIG. 12A), pHWO₁₂-GFP, pHWO₁₃-sfGFP and pHWO₃-sfGFP) and/or the E. coli rrnB gene terminators (pHWT₁T₂-sfGFP and pHWO₁₂₃T₁T₂-sfGFP (FIG. 12B)) and cloned into the SnaBI/Nael sites of the pHW plasmid (GenScript USA). The avian N1 (1999 NA; GenBank ID: CY016957) and the HA (H1-BR18 and the H6) gene segments together with their 5′ and 3′ UTR's were cloned into the modified pHW plasmids by replacement of the sfGFP gene using the simplified Gibson assembly method.

### Transformation and Colony Screening

Ligation reactions consisting of 1 μl of the PCR insert and vector mixtures were transformed into 50 μl of XL10-Gold cells per the manufacturer's instructions (Agilent) and cultured overnight at 37° C. on LB+ampicillin agar plates. Agar plates were imaged with an Azure C600 and 5-10 individual or pooled colonies were randomly selected for growth on a master plate and for direct colony screening by PCR. For screening, colonies were resuspended in 1x PCR reaction buffer (RB) (10×RB: 200 mM Tris-HCl, 100 mM KCl, 60 mM (NH₄)₂SO₄, 20 mM MgSO₄, 1 mg/ml BSA and 1% Triton; pH 8.8) for lysis and the DNA was amplified over 30 cycles using Pfu X7 DNA polymerase and a primer pair targeting the plasmid (pHW FWD Screening Primer) and the specific insert (NA/HA Reverse Primer). The amplified DNA was analyzed by agarose gel (0.8%) electrophoresis. Overnight liquid cultures (LB broth) were used to amplify the positive clones for additional studies including virus rescue. Plasmid DNA was isolated using the QIAprep Spin Miniprep Kit (Qiagen) and all constructs were sequenced prior to use (Macrogen).

sfGFP Expression in E. Coli and Fluorescence-Detection Size Exclusion Chromatography

Plasmids containing sfGFP were transformed into XL10 Gold cells and amplified overnight in 10 ml LB broth cultures containing 100 μg/mL ampicillin. The following day, 1 ml of the overnight culture was sedimented (10,000×g; 5 min) and the bacterial pellets were resuspended in 1 ml lysis buffer (50 mM Tris-HCl pH 7.0, 150 mM NaCl, 1 mM MgCl₂, 200 μg/ml lysozyme, 1x EDTA-free protease inhibitors, spec DNase I), incubated for 30 mins at room temperature and sonicated on ice (5 s×6; amplitude 10%). The sonicated lysates were sedimented (6,000×g; 1 min) to remove insoluble debris and analyzed by fluorescent size exclusion chromatography (FSEC) using an Agilent 1260 prime HPLC equipped with an AdvanceBio SEC 300Å column and a fluorescent detector set at 486 nm excitation and 524 nm emission wavelengths. A protein standard (AdvanceBio SEC 300Å protein standard; Agilent) of known molecular weight was included in each run to estimate the molecular weight/stokes radius of the expressed sfGFP.

### Cell Culture and GFP Expression Analysis in Eukaryotic Cells

HEK 293T/17 cells (CRL-11268) were cultured at 37° C. with 5% CO₂ and ˜95% humidity in DMEM containing 10% FBS and 100 U/ml P/S. For each transfection, ˜7.5×10⁵ HEK cells in DMEM containing 10% FBS were seeded in a 12-well plate. When the wells reached 75-80% confluency, ˜24 hours post seeding, 1.0 μg of each pHW plasmid encoding sfGFP was separately added to 100 ml of OMEM, mixed with 3 μl of TransIT-LT1 transfection reagent, and incubated for 30 minutes at room temperature before addition to a well containing the HEK cells. Live-cell imaging for GFP expression was performed ˜60 hours post-transfection using a Keyence BZ-X810 fluorescence microscope with a 10x objective and a BZ-X GFP cube filter (470 nm excitation and 525 nm emission wavelengths). Image capture settings were fixed across the experiment. Post-imaging, the cells in each well were harvested in 1 ml 1xPBS, sedimented (6,000×g; 1 minute), and resuspended in 150 ml lysis buffer (50 mM Tris-HCl pH 7.0, 150 mM NaCl, 0.5% n-Dodecyl-B-D-Maltoside (DDM), and 1xEDTA-free protease inhibitors). The lysed samples were sedimented (6,000×g; 1 minute) to obtain a post-nuclear supernatant. GFP relative fluorescence units (RFUs) in each post-nuclear supernatant (100 ml) were measured in a 96-well low protein binding black clear bottom plate (Corning) on a Cytation 5 (Biotek) plate reader with 485 nm excitation and 528 nm emission wavelengths.

### Viral Reverse Genetics

Madin-Darby canine kidney 2 (MDCK.2; CRL-2936) cells and HEK 293T/17 cells (CRL-11268) were cultured at 37° C. with 5% CO₂ and ˜95% humidity in DMEM containing 10% FBS and 100 U/ml P/S. Reassortant viruses were created by 8-plasmid reverse genetics in T25 flasks using the indicated NA, or NA and HA pair, and the complimentary seven, or six, gene segments of WSN. For each virus, ˜1.5×10⁶MDCK.2 cells in OMEM containing 10% FBS were seeded in a T25 flask and allowed to adhere for 45 mins. During this period, the eight RG plasmids (1.5 μg of each) were added to 750 μl of serum-free OMEM, mixed with 24 μl of TransIT-LT1 transfection reagent, and incubated 20 min at room temperature. A 750μl suspension of 293T/17 cells (˜3×10⁶/ml) in serum-free OMEM was added to each transfection mixture and incubated for 10 minutes at room temperature before addition to the T25 flask containing the MDCK.2 cells. At ˜24 h post-transfection, the media in each flask was replaced with 3.5 ml of DMEM containing 0.1% FBS, 0.3% BSA, 4 μg/ml TPCK trypsin, 1% P/S and 1% L-glutamine. NA activity and HAU measurements were taken immediately following transfection and every 24 h until viral harvest. Rescued viruses in the culture medium were harvested 72-96 h post-transfection, clarified by sedimentation (2,000×g; 5 min) and passaged in SPF eggs.

### Viral Passaging in SPF Chicken Eggs

Initial passages (E1) were carried out by inoculating 9-11 day old embryonic SPF chicken eggs with 100 μl of the rescued virus diluted 1/10 in PBS. Eggs were incubated for 3 days at 33° C. and placed at 4° C. for 2 h prior to harvesting. Allantoic fluid was harvested individually from each egg and clarified by sedimentation (2,000×g; 5 min). NA activity and HAU measurements were taken prior to combining each viral harvest for storage at −80° C. or viral purification.

### Viral Purification

Viruses in allantoic fluid were isolated by sedimentation (100,000×g; 45 min) at 4° C. through a sucrose cushion (25% w/v sucrose, PBS pH 7.2 and 1 mM CaCl₂) equal to 12.5% of the sample volume. The supernatant was discarded, the sedimented virions were resuspended in 250 μl PBS pH 7.2 containing 1 mM CaCl₂ and the total protein concentration was determined using a BCA protein assay kit (Pierce). All purified viruses were adjusted to a concentration of ˜500 μg/ml using PBS pH 7.2 containing 1 mM CaCl₂ prior to analysis on a 4-12% SDS-PAGE gel.

NA activity, HAU and Viral Titer Measurements

All NA activity measurements were performed in a 96-well low protein binding black clear bottom plate (Corning). Each sample (50 μl viral cell-culture medium or 10 μl allantoic fluid) was mixed with 37° C. reaction buffer (0.1 M KH₂PO₄ pH 6.0 and 1 mM CaCl₂) to a volume of 195 μl. Reactions were initiated by adding 5 μl of 2 mM MUNANA and the fluorescence was measured on a Cytation 5 (Biotek) plate reader at 37° C. for 10 minutes using 30-second intervals and a 365 nm excitation wavelength and a 450 nm emission wavelength. Final activities were determined based on the slopes of the early linear region of the relative fluorescent units (RFU) versus time graph.

HAU titers were determined by a two-fold serial dilution in 96-well plates using a sample volume of 50 μl and PBS pH 7.4. Following the dilution, 50 μl of 0.5% TRBCs were added to each well and the plate was incubated 30 minutes at room temperature. HAU titers were determined as the last well where agglutination was observed. Median tissue culture infectious doses (TCID₅₀) per milliliter and median egg infectious doses (EID₅₀) per milliliter were calculated using 100 μL inoculums of MDCK cells and SPF eggs as previously described (Reed and Muench, Am J Epidemiol 27:493-497, 1938). MDCK cell cytopathic effects and egg infections were verified by the presence of NA activity.

### SDS-PAGE, Coomassie Staining

Purified virions equal to ˜5 μg of total viral protein were mixed with 2× sample buffer. Samples were heated at 50° C. for 10 minutes and resolved on a 4-12% polyacrylamide Tris-Glycine SDS-PAGE wedge gel. Gels were stained with simple blue and imaged with an Azure C600.

### Example 2: Construction of Human and Avian H1N1 NA Plasmid Libraries

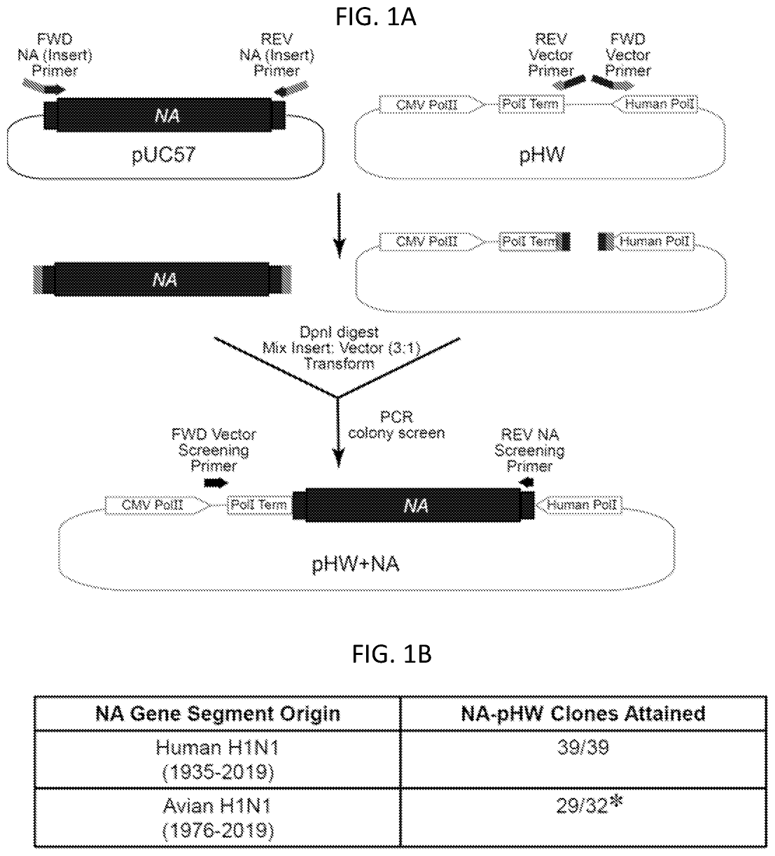

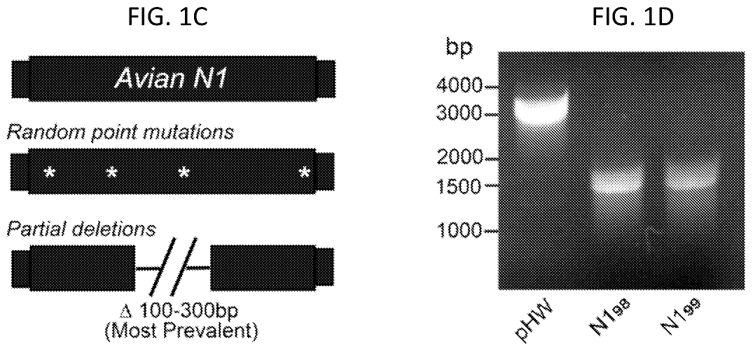

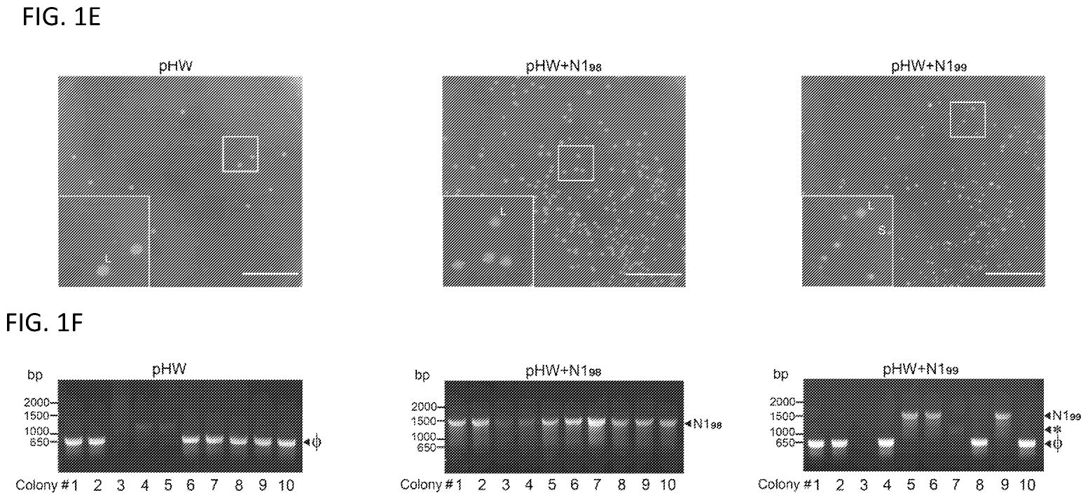

To assess temporal and species related changes in the properties of NA from influenza A viruses, a reverse genetics plasmid library carrying NA genes from human and avian H1N1 viruses isolated throughout the last century was generated. The library was created by a modified Gibson assembly method where the NA subtype 1 (N1) genes were inserted between the human polymerase I (Pol I) and cytomegalovirus polymerase II (CMV Pol II) promoters of the common influenza reverse genetics plasmid (pHW) (FIG. 1A and Hoffmann et al., Proc Natl Acad Sci USA 97(11):6108-6113, 2000). During construction of the library, three of the avian N1s (1983, 1991 and 1999) proved difficult to clone into the pHW plasmid (FIG. 1B). After more extensive screening, putative clones were obtainable for these NAs. However, the isolated plasmids typically possessed either point mutations, insertions or heterogeneity in different regions of the NA genes (FIG. 1C and FIGS. 8A-8C), indicating the genes may be unstable in E. coli.

The cloning problem was examined more thoroughly by comparing the problematic avian N1 gene from 1999 (N1₉₉) to the more easily cloned avian N1 gene from 1998 (N1₉₈). Although no difficulties were observed in amplifying each NA gene segment or the pHW plasmid (FIG. 1D), transformation with a mixture containing the amplified N1₉₉ gene and pHW vector (pHW+N1₉₉) yielded atypical E. coli colonies on agar plates that were much smaller than the colonies transformed with a mixture of pHW+N1₉₈ (FIG. 1E). PCR screening of randomly selected large and small colonies from the plates showed that all the colonies transformed with pHW+N1₉₈ produced a band corresponding with the expected full-length NA gene insert (FIG. 1F, middle panel), whereas only a minority (30%) of the colonies transformed with pHW+N1₉₉ yielded the expected ˜1500 bp band (FIG. 1F, right panel). Subsequent sequencing of the plasmids revealed that the few potential full-length pHW-N1₉₉ clones often contained multiple point mutations, rendering them unsuitable.

### Example 3: Analysis of Gene Expression from the pHW Influenza Reverse Genetics Plasmid in E. Coli

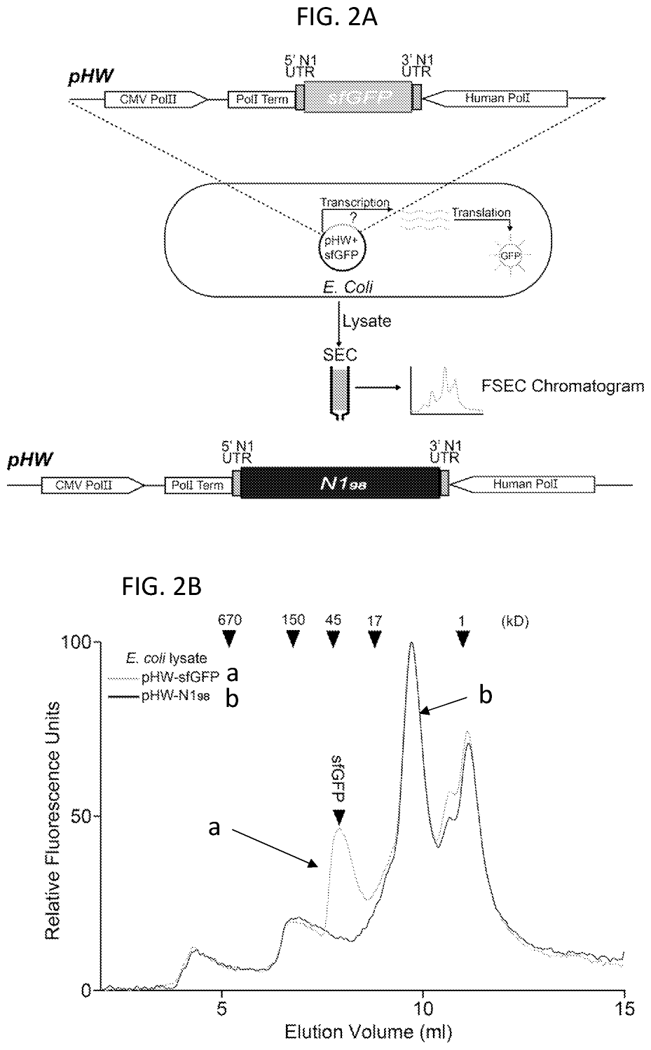

Previous studies have shown that the CMV Pol II promoter in eukaryotic expression plasmids contains E. coli promoter-like sequences. Therefore, it was hypothesized that E. coli promoter-like sequences in the CMV Pol II promoter of the pHW plasmid leads to expression of influenza genes in E. coli, which can potentially be toxic to the bacteria. To test this hypothesis, E. coli were transformed with a pHW reporter plasmid (pHW-sfGFP) that encodes the robust super folder green fluorescent protein (sfGFP) (Pedelacq et al., Nat Biotechnol 24:79-88, 2006; Drew et al., Nat Methods 3:303-313, 2006; Schlegel et al., Cell Rep 10(10):1758-1766, 2015), and a control plasmid pHW-N1₉₈ expressing a stable NA gene (FIG. 2A). Whole cell bacterial lysates were then prepared and analyzed by fluorescent size exclusion chromatography (FSEC) to monitor sfGFP expression (FIG. 2A; and Kawate and Gouaux, Structure 14:673-681, 2006) because the common approaches of monitoring colony fluorescence by imaging the plate or measuring E. coli fluorescence in suspension by a plate reader were not sensitive enough to overcome the signal from the inherent fluorescent molecules in E. coli. Following the FSEC analysis, a clear peak corresponding to sfGFP was only observed from the bacteria transformed with pHW-sfGFP (FIG. 2B), indicating that genes inserted into the pHW plasmid are expressed in E. coli at a detectable level.

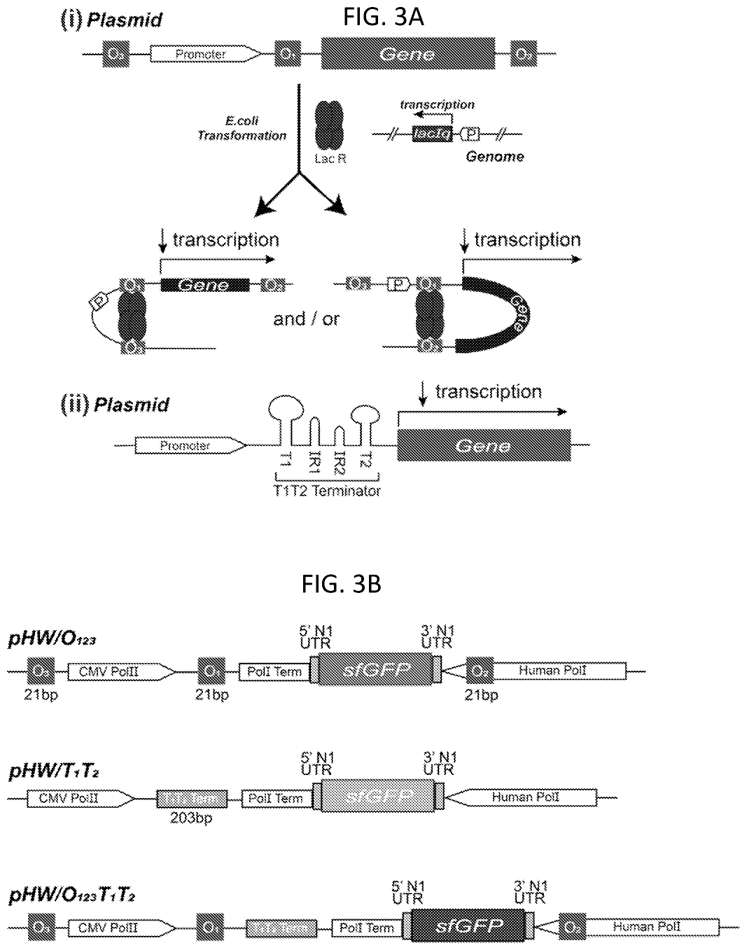

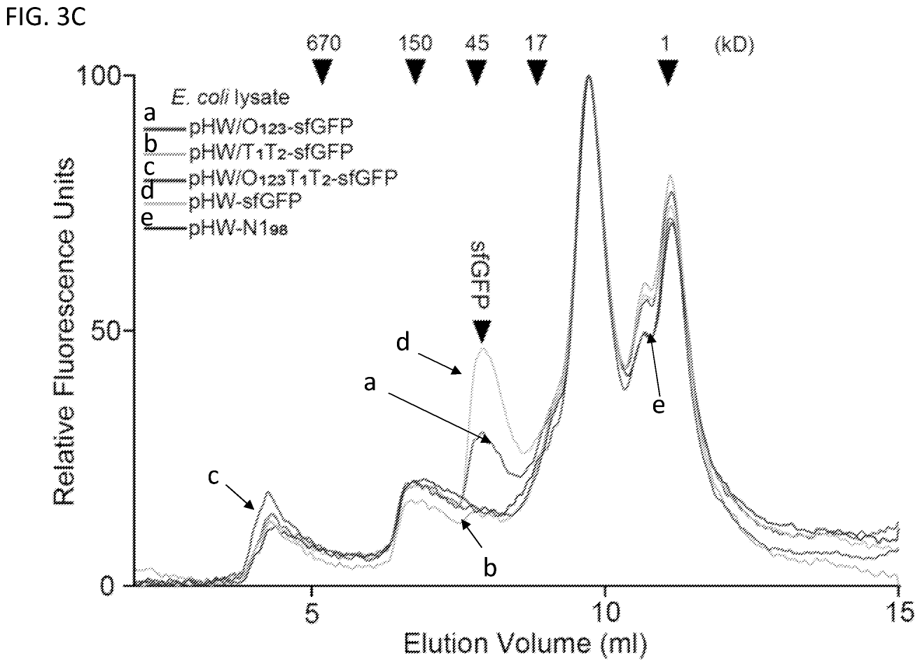

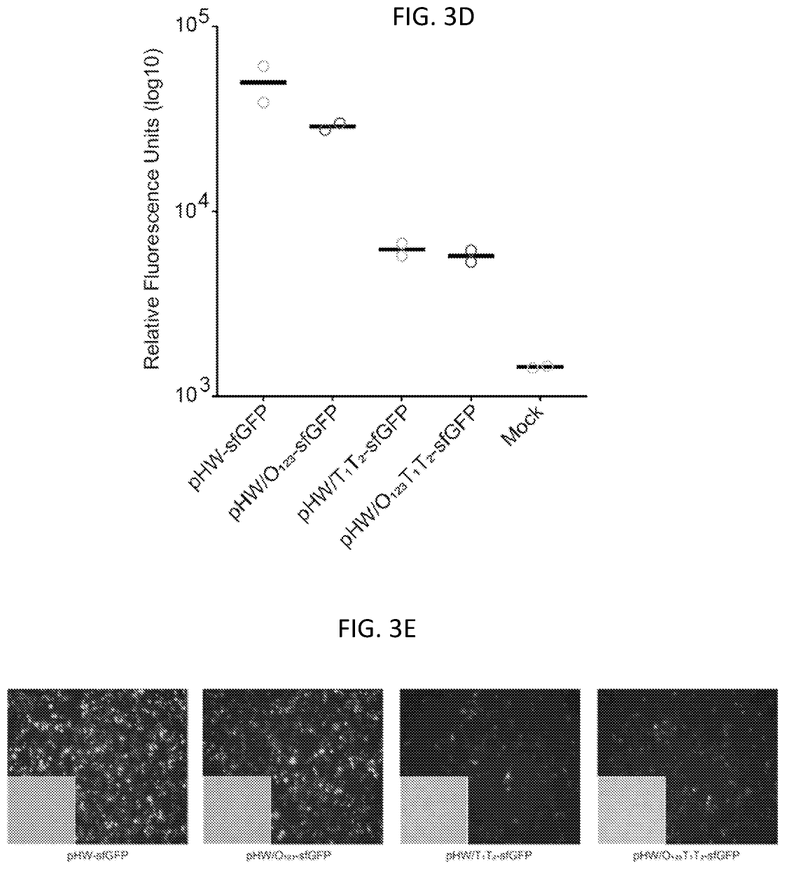

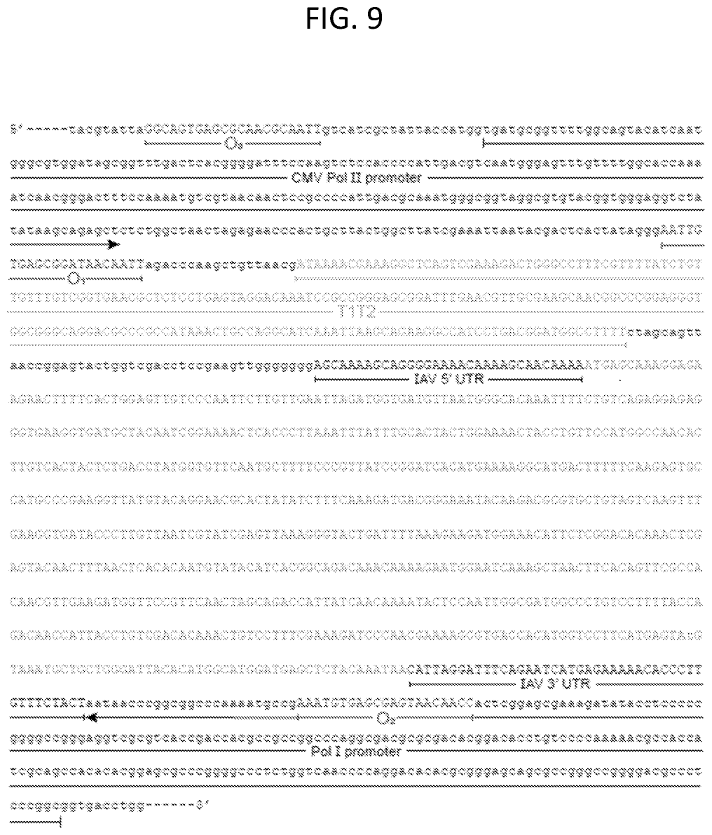

Based on the sfGFP results, attempts were made to abrogate gene expression from the pHW plasmid in E. coli by two approaches (FIG. 3A and FIG. 9). The first involved cooperative repression using the three regulatory elements (operators) from the E. coli lac operon while the second approach made use of the efficient transcription termination region of the E. coli rrnB gene. Three pHW variant plasmids were constructed: the first contained the three natural lac operator sequences (FIG. 3B, pHW/O₁₂₃; SEQ ID NO: 6), with one operator positioned on each side of the CMV Pol II promoter sequence and the last operator positioned downstream of the sfGFP gene; in the second, the transcription termination region of the E. coli rrnB gene was inserted downstream of the CMV Pol II promoter sequence (FIG. 3B, pHW/T₁T₂); and for the final construct both of the regulatory elements were inserted into a single pHW plasmid (FIG. 3B, pHW/O₁₂₃T₁T_{2;}; SEQ ID NO: 7). Compared to lysates from bacteria transformed with the pHW-sfGFP plasmid, the GFP signal was reduced by ˜50% in the bacteria transformed with pHW/O₁₂₃-sfGFP and by ˜95% in bacteria transformed with pHW/T₁T₂-sfGFP (FIG. 3D). The GFP signal was further reduced in the bacteria transformed with pHW/O₁₂₃T₁T₂-sfGFP as the FSEC trace was indistinguishable from the negative control (FIG. 3D).

To determine if the presence of the lac operators or the transcriptional terminators hindered gene expression driven by the CMV Pol II promoter, 293T cells were transfected with each of the plasmids. The transfected cell lysate fluorescence (FIG. 3D) and live cell imaging (FIG. 3E) both showed that the GFP signal in the pHW/O₁₂₃-sfGFP transfected cells was ˜60% of the pHW-sfGFP transfected cells. In the cells transfected with either pHW/T₁T₂-sfGFP or pHW/O₁₂₃T₁T₂-sfGFP the GFP signal was reduced to ˜10% of the pHW-sfGFP transfected cells, indicating that the lac operators have less of an impact on the mRNA transcription from the plasmid in eukaryotic cells.

### Example 4: Stability of the Avian N1₉₉ Gene Segment in the Modified pHW Plasmids

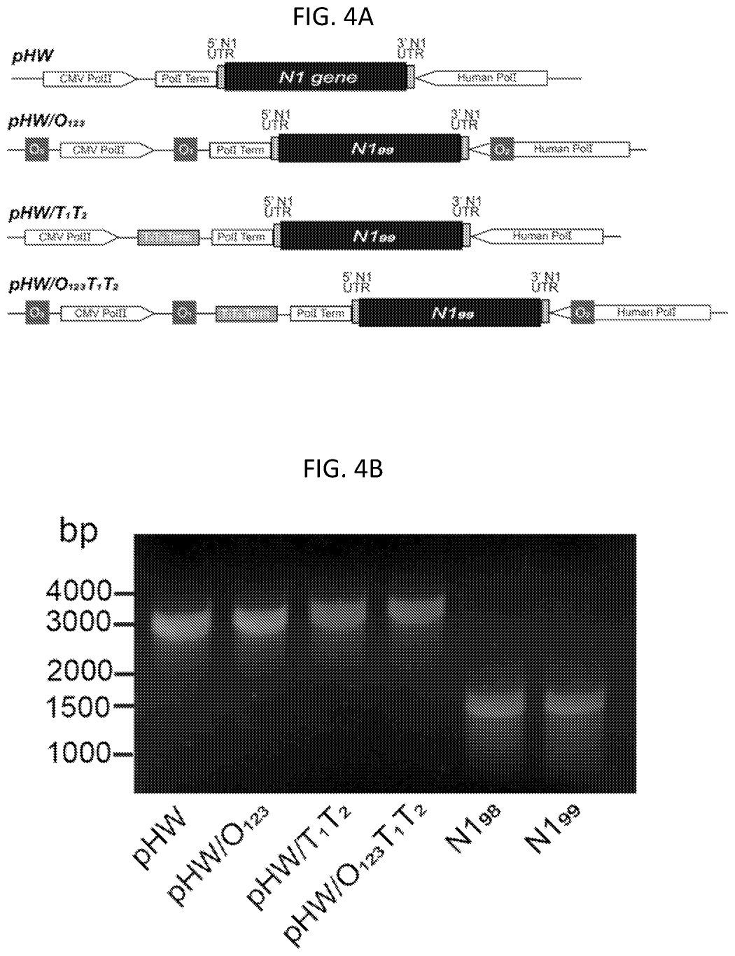

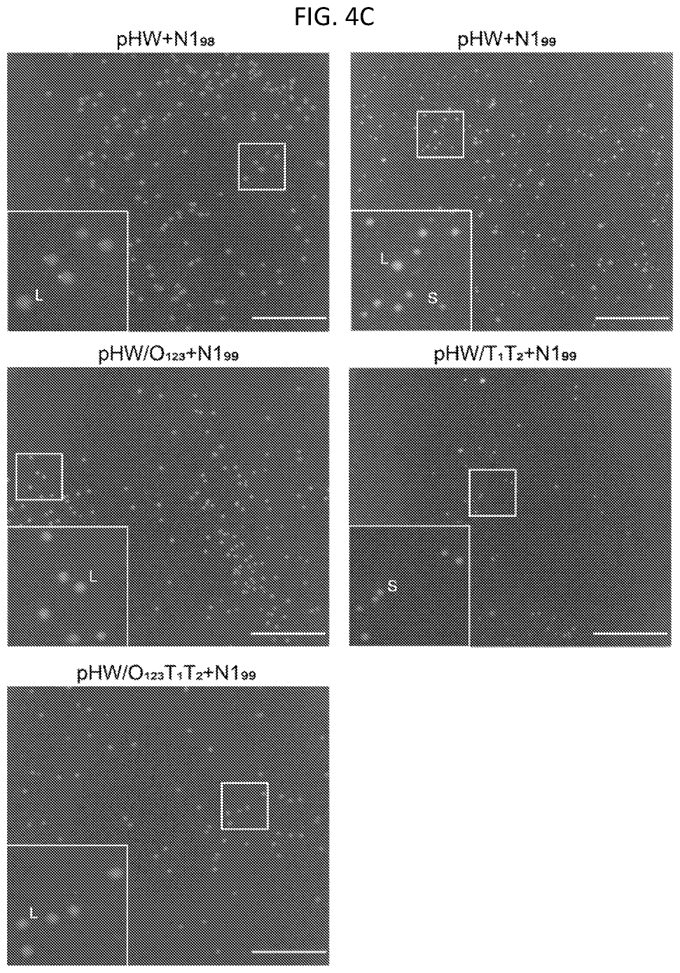

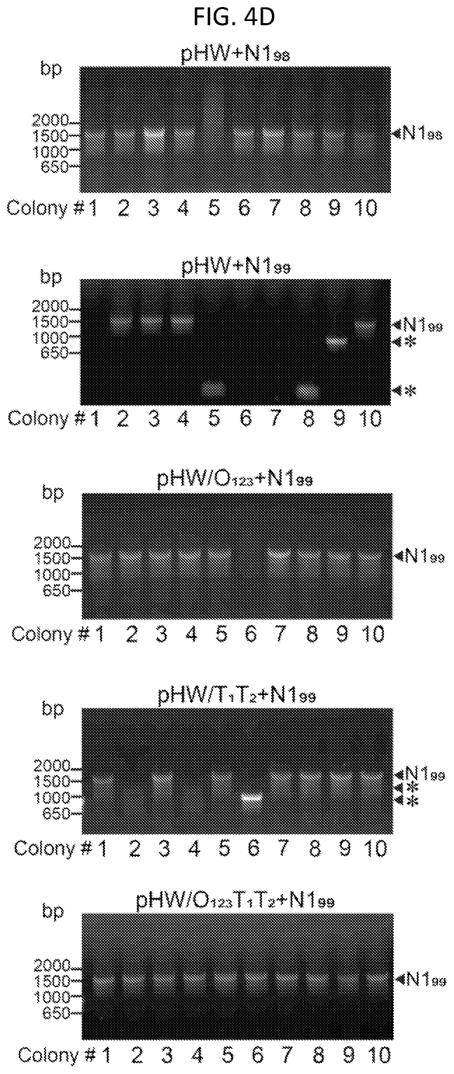

To test if the regulatory elements improved the ability to clone the problematic avian N1₉₉ gene, the cloning results using the pHW plasmid were compared with the three modified pHW plasmids (FIG. 4A). Despite the presence of highly structured terminators, no difficulties were observed in amplifying the three modified pHW plasmids by PCR (FIG. 4B). Transformations with the pHW/O₁₂₃+N1₉₉ and the pHW/O₁₂₃T₁T₂+N1₉₉ mixtures both produced E. coli colonies that were larger than the pHW+N1₉₉ colonies and more similar in size to the colonies obtained from the negative control transformation with pHW+N1₉₈ (FIG. 4C). The colonies from the E. coli transformed with the pHW/T₁T₂+N1₉₉ mixture remained small (FIG. 4C), which was unexpected based on the sfGFP results. The phenotypic observations were supported by a PCR screen of randomly selected large and small colonies as almost 100% of the pHW/O₁₂₃+N1₉₉ and pHW/O₁₂₃T₁T₂+N1₉₉ colonies yielded a band corresponding to the full-length N1₉₉ insert, whereas only 65% of the pHW/T₁T₂+N1₉₉ colonies and 52% of the pHW+N1₉₉ colonies yielded the expected band (FIG. 4D and Table 2). The high positive screen rates for pHW/O₁₂₃+N1₉₉ and pHW/O₁₂₃T₁T₂+N1₉₉ were in line with the 94% positivity rate for the negative control transformation with pHW+N1₉₈ (FIG. 4D and Table 2), demonstrating that the lac operators effectively minimize the bacterial toxicity caused by the NA gene in the pHW plasmid.

TABLE 2
Colony screening of the NA gene segments
cloned into the pHW plasmid variants

| Plasmid | Positive full-length clones |
|---|---|
| pHW + N1₉₈ | 31/33 (93.9%) |
| pHW + N1₉₉ | 17/33 (51.5%) |
| pHW/O₁₂₃ + N1₉₉ | 21/23 (91.3%) |
| pHW/T1T2 + N1₉₉ | 15/23 (65.2%) |
| pHW/O₁₂₃T1T2 + N1₉₉ | 23/23 (100%) |

NA gene segments and the indicated pHW plasmid variant were amplified by PCR, mixed and transformed into E. coli.
Putative positive full-length clones were determined by a PCR screen of randomly selected colonies using a primer pair that targets an upstream region in the plasmid (pHW Screen) and the 3′ end of the NA insert (NA Reverse).

### Example 5: Stability of HA Gene Segments in the Modified pHW Plasmids

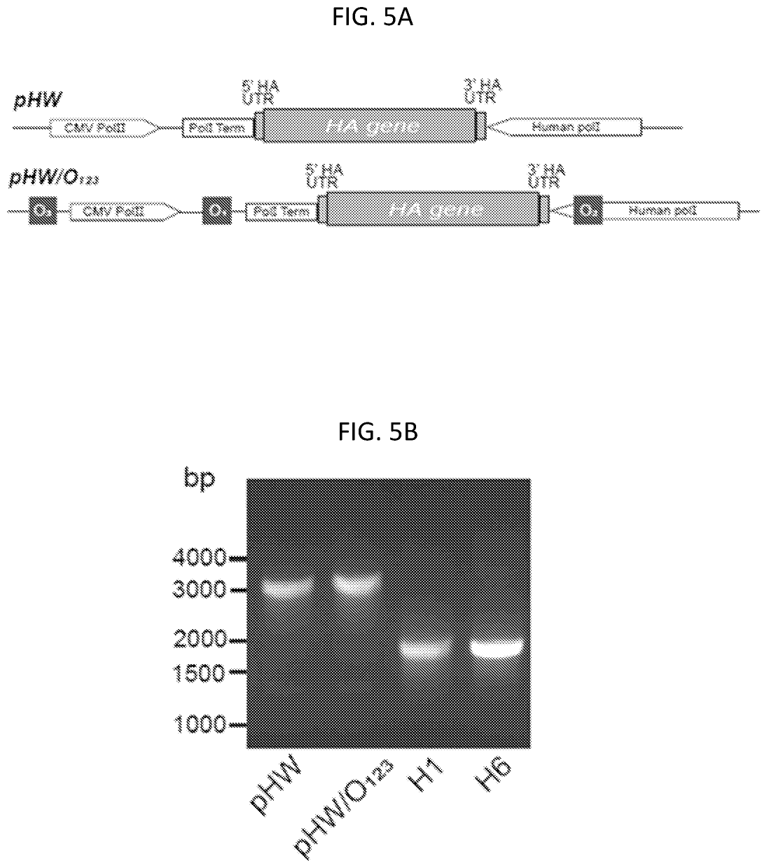

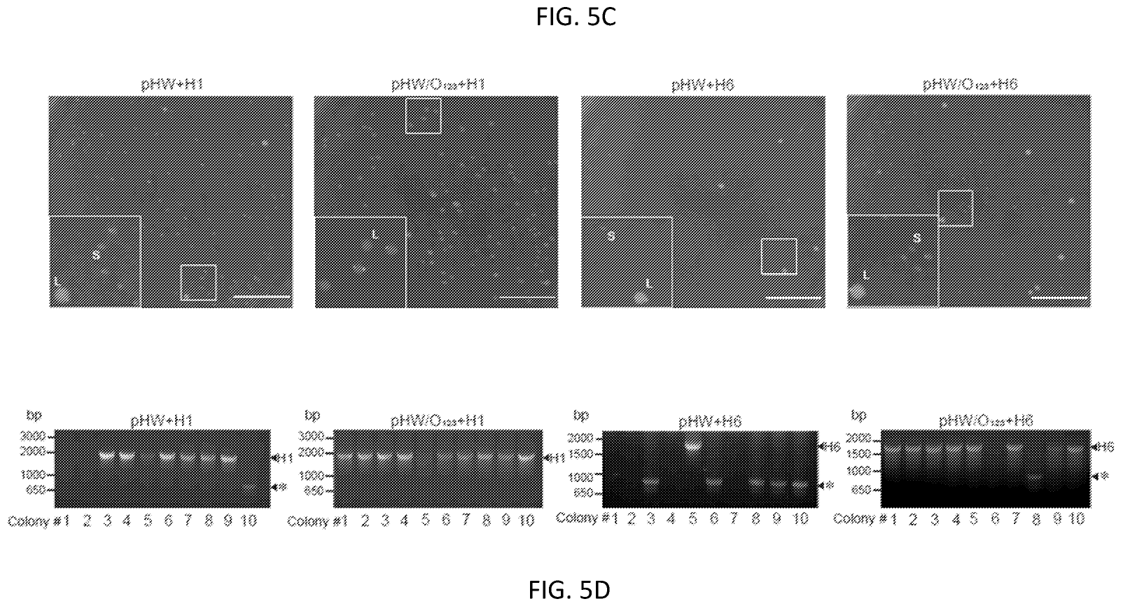

Prior studies demonstrated problems cloning two different HA (H1 and H6) gene segments into the pHW plasmid (FIG. 10). Thus, these genes were tested to demonstrate the ability of the pHW/O₁₂₃ plasmid (SEQ ID NO: 8) to increase the stability of influenza genes (FIG. 5A). As expected, no difficulties were observed in amplifying the pHW/O₁₂₃ plasmid or the H1 and H6 gene segments (FIG. 5B). Following transformation with pHW/O₁₂₃+H1, the colonies were noticeably larger than those transformed with pHW+H1 (FIG. 5C, top two panels). For the H6 gene, the results were somewhat different. Transformation with pHW/O₁₂₃+H6 yielded numerous small colonies whereas the transformation with pHW+H6 produced very few large colonies and almost no small colonies (FIG. 5C, bottom two panels). Random PCR colony screening showed that 100% of the colonies transformed with pHW/O₁₂₃+H1 and 87% of the colonies transformed with pHW/O₁₂₃+H6 produced products of the expected length, compared to 70% for the pHW+H1 colonies and 15% for the pHW+H6 colonies (Table 3 and FIG. 5D). These screening results combined with the phenotypic observations confirmed that the lac operators in pHW can also minimize HA gene toxicity, likely by silencing expression of the HA gene from the plasmid in E. coli.

TABLE 3
Colony screening of the HA gene segments
cloned into the pHW and pHW/O₁₂₃ plasmids

| Plasmid | Positive full-length clones |
|---|---|
| pHW + H1 | 7/10 (70.0%) |
| pHW/O₁₂₃ + H1 | 10/10 (100.0%) |
| pHW + H6 | 2/13 (15.4%) |
| pHW/O₁₂₃ + H6 | 13/15 (86.7%) |

HA gene segments and the indicated pHW plasmid were amplified by PCR, mixed and transformed into E. coli.
Putative positive full-length clones were determined by a PCR screen of randomly selected colonies using a primer pair that targets an upstream region in the plasmid (pHW Screen) and the 3′ end of the HA insert (HA Reverse).

### Example 6: Dependence of H6 Gene Segment Stability on the lac Operator Locations in pHW/O₁₂₃

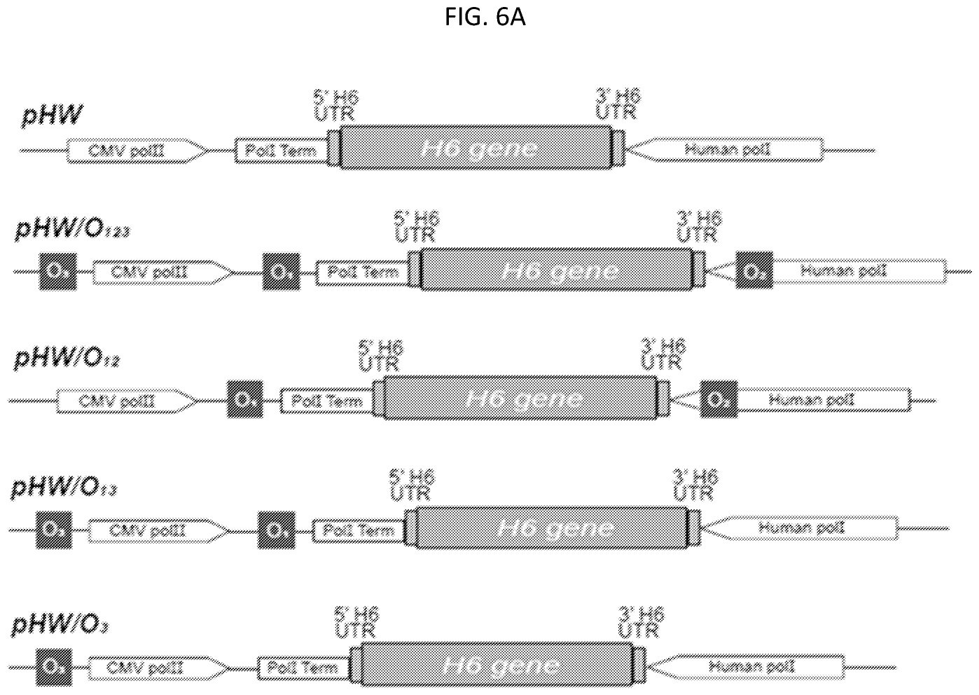

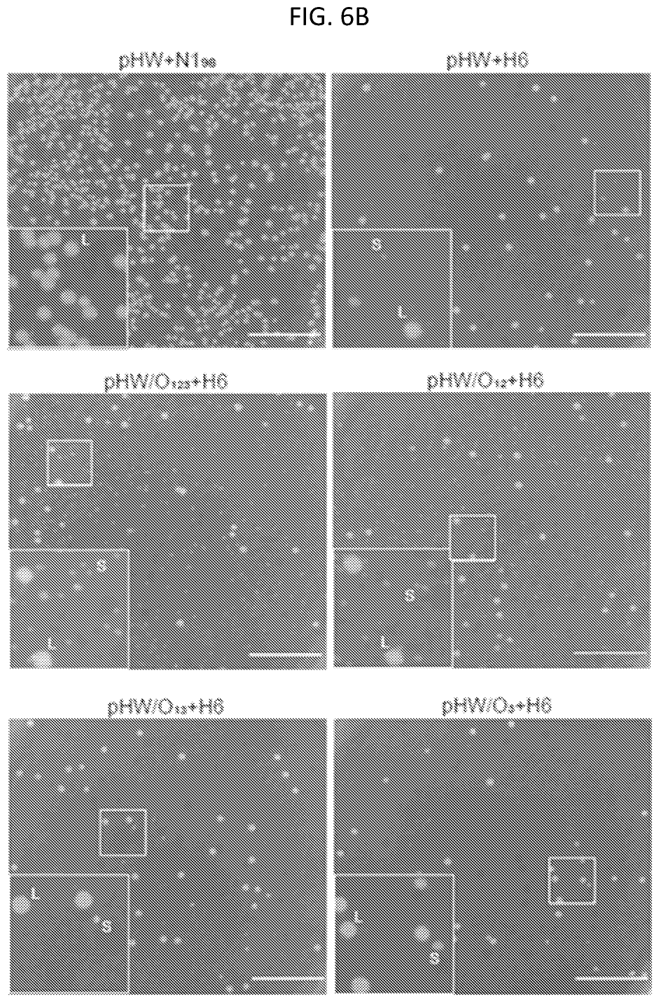

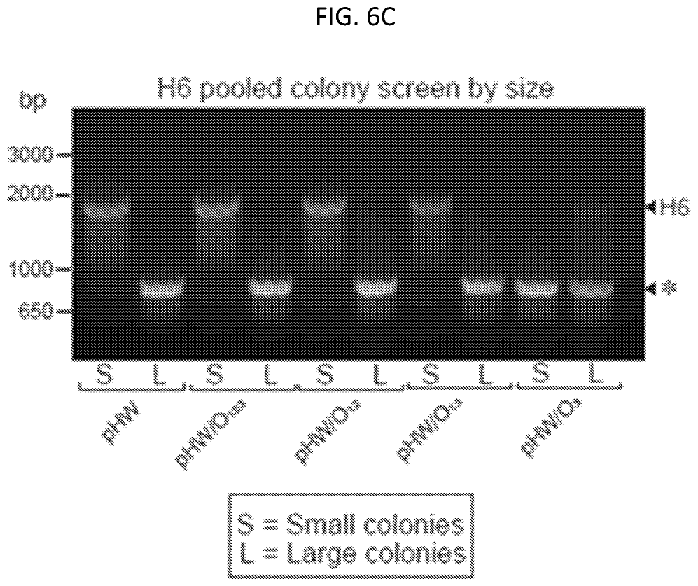

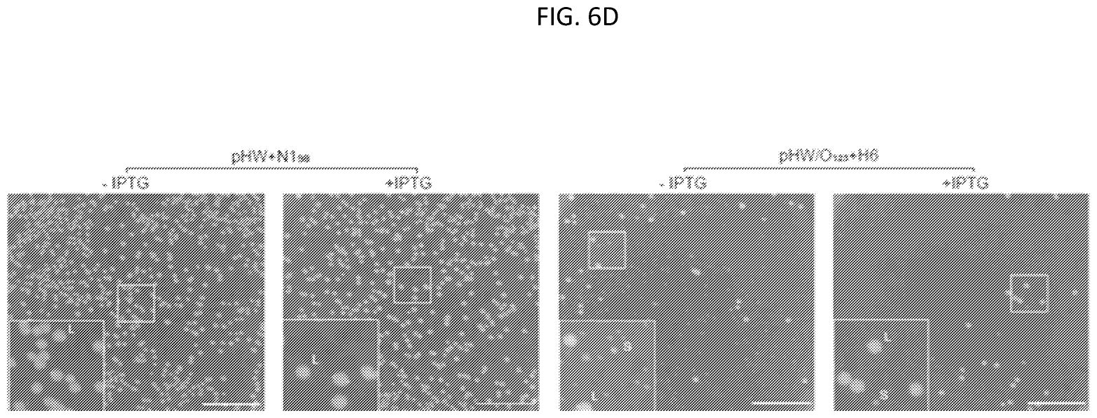

To investigate if all three lac operators are essential for H6 gene segment stability, three additional variants of the pHW/O₁₂₃ plasmid were created (FIG. 6A). One contained two operators located on either side of the gene segment insert (pHW/O₁₂; SEQ ID NO: 10), the second contained two operators on either side of the CMV Pol II promoter sequence (pHW/O₁₃) and the third was a control that contained only one operator upstream of the CMV Pol II promoter sequence (pHW/O₃). Transformations with pHW/O₁₃+H6 and pHW/O₃+H6 both yielded few large colonies similar to pHW+H6. In contrast, transformations with pHW/O₁₂+H6 (SEQ ID NO: 10) produced numerous small colonies, similar to pHW/O₁₂₃+H6 (SEQ ID NO: 8), indicating that the operators on both sides of the H6 gene segment provide the best stability (FIG. 6B). As expected, the control transformation with pHW+N1₉₈ yielded mostly large bacterial colonies. Next, five pooled large and five pooled small colonies from each plate were screened for the presence of the full-length H6 insert by PCR (FIG. 6C). In all but one instance (pHW/O₃+H6), the pooled small colonies produced a band at the expected size for full-length H6, whereas all the pooled large colony preparations yielded a much smaller band. Taken together, these data suggest that small colony size is related to H6 gene segment toxicity and that the toxicity is likely due to cryptic promoter-like sequences within the H6 open reading frame (ORF) or the untranslated regions (UTRs) rather than the pHW plasmid.

The data indicated that the expression from the influenza genes in the pHW plasmid is responsible for the observed toxicity and cloning difficulties. To test this more directly, a study was performed to take advantage of the ability to regulate the Lac repressor by plating an equivalent portion of pHW/O₁₂₃+H6 transformed bacteria on plates that lacked or contained IPTG (FIG. 6D). Similar to previous results, the E. coli transformed with pHW/O₁₂₃+H6 and grown on agar plates lacking IPTG yielded numerous small colonies. However, when the same E. coli were grown on agar plates containing IPTG only very few large colonies were observed, similar to the transformations with pHW+H6. The non-toxic pHW+N1₉₈ transformed bacteria was included as a control and no differences in colony morphology were observed on either agar plate. These results demonstrate that the Lac repressor mediated repression of expression from the H6 ORF when cloned in the pHW/O₁₂₃ plasmid is important for the genetic stability of toxic genes.

### Example 7: Influenza Virus Rescue Using the Modified pHW/O₁₂₃ Plasmid

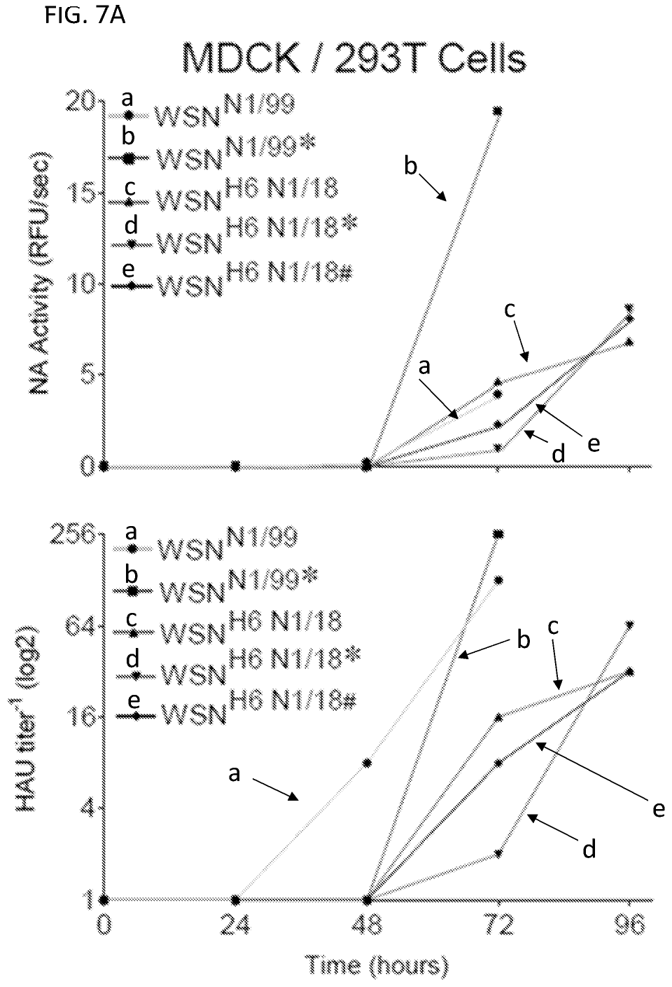

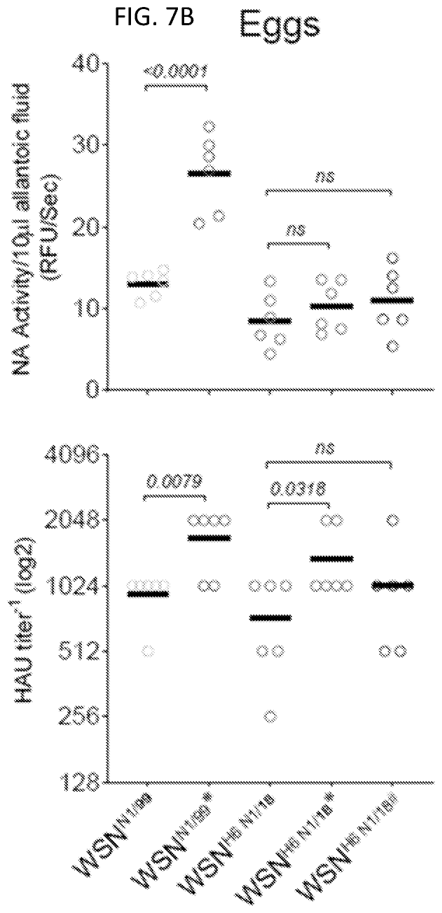

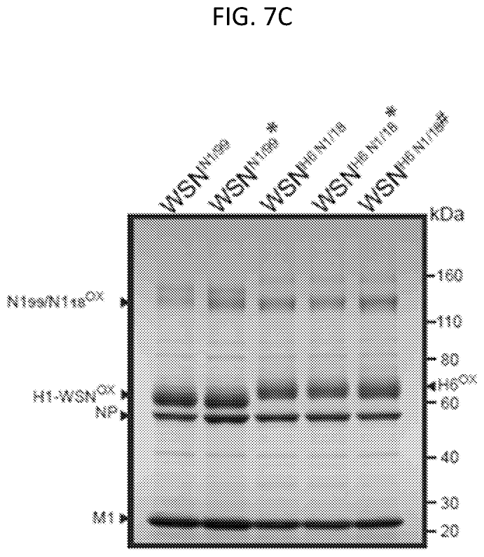

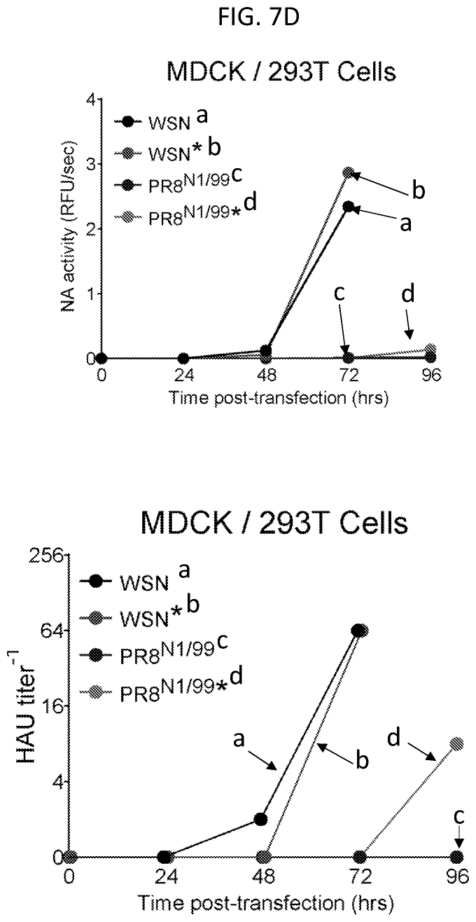

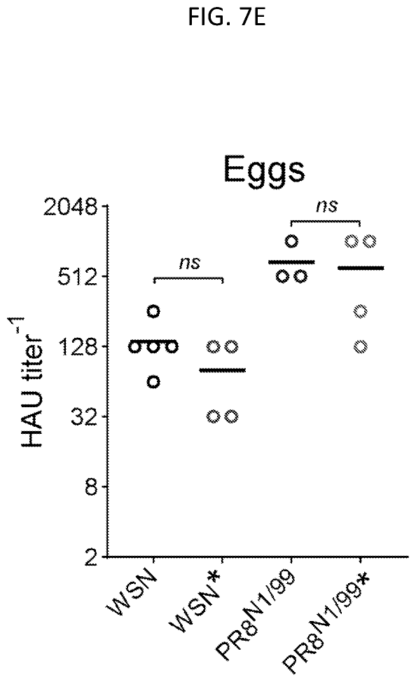

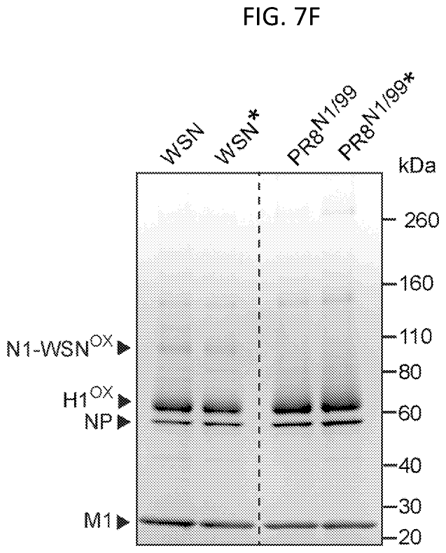

Addition of two or more lac operators in the pHW plasmid made the largest contribution to stability (FIGS. 4D and 5D) and showed the least impact on expression in mammalian cells (FIG. 3E). Therefore, the viral rescue kinetics from the pHW/O₁₂₃ plasmid was compared to the parental pHW plasmid. For the initial analysis, viruses were generated using either the pHW/O₁₂₃-N1₉₉ or pHW/O₁₂₃-H6 plasmids together with seven pHW backbone plasmids that encode for gene segments from the H1N1 IAV strain A/WSN/1933 (WSN). Both viruses generated with a pHW/O₁₂₃ plasmid (WSN^{N1/99}* and WSN^{H6 N1/18}*) showed a slight delay in the production of NA activity and HAU titers (FIG. 7A). However, at later time points, NA activity and HAU titers equaled or exceeded those for the viruses (WSN^{N1/99} and WSN^{H6 N1/18}) generated exclusively with pHW plasmids, indicating that viruses rescued from the pHW/O₁₂₃ plasmid reached similar titers to pHW rescued viruses despite the slightly slower kinetics. The same pHW-H6 and pHW/O₁₂₃-H6 plasmids were sent for large scale DNA production to determine if the plasmids could be propagated in an independent lab setting. Virus (WSN^{H6N1/18 #}) was successfully rescued from the commercial pHW/O₁₂₃-H6 plasmid DNA, which contained the correct H6 sequence (FIG. 7A). In contrast, the commercial pHW-H6 plasmid DNA contained an insertion in the H6 gene making it unsuitable for virus rescue, further confirming that influenza genes are more stable in the pHW/O₁₂₃ plasmid.

All rescued viruses were passaged in embryonated eggs to determine if any differences were observed in viral propagation or protein content. Each virus rescued from the pHW/O₁₂₃ plasmid preparations (WSN^{N1/99}*, WSN^{H6 N1/18}*, and WSN^{H6 N1/18 #}) produced NA activities, HAU, and infectious titers that were equivalent or higher than the analogous viruses (WSN^{N1/99} and WSN^{H6 N1/18}) produced entirely from pHW plasmids (FIG. 7B and Table 4). SDS-PAGE analysis of the isolated viruses showed that the viral protein content was similar and that the H6 and N1₉₉ proteins resolved at the expected molecular weights (FIG. 7C), indicating pHW/O₁₂₃ retains the ability to produce recombinant virus.

TABLE 4
Infectious titers of rescued viruses
following a single passage in eggs^{a}

| Rescued Virus | TCID₅₀/mL | EID₅₀/mL |
|---|---|---|
| WSN^{N1/99} | 1.4 × 10⁶ | n.d. |
| WSN^{N1/99}* | 2.3 × 10⁷ | n.d. |
| WSN^{H6} ^{N1/18} | 3.3 × 10⁴ | 6.8 × 10⁷ |
| WSN^{H6} ^{N1/18}* | 5.3 × 10⁴ | 6.8 × 10⁷ |
| WSN^{H6} ^{N1/18#} | 2.5 × 10⁴ | n.d. |

^{a}Median tissue culture infectious doses per milliliter (TCID₅₀/mL) were determined using MDCK cells in 96-well plates, and the results represent the mean of two independent analysis.
Median egg infectious doses per milliliter (EID₅₀/mL) were determined using specific pathogen-free eggs.
Asterisks indicate viruses rescued from the pHW/O₁₂₃-N1/₉₉ and pHW/O₁₂₃-H6 plasmids, respectively.
The hashtag (#) represents the virus rescued from a commercial preparation of pHW/O₁₂₃-H6.
n.d. = not determined.

To examine if the delay in the viral rescue kinetics would be exacerbated in other settings, the rescue of WSN from eight pHW/O₁₂₃ plasmids versus the eight parental pHW plasmids was compared. In addition, viral rescue from the pHW/O₁₂₃-N1₉₉ plasmid was compared to viral rescue from the pHW-N1₉₉ plasmid in combination with seven different pHW backbone plasmids from the H1N1 IAV strain A/PR/8/1934 (PR8). During the rescue, the WSN viruses generated by the eight pHW/O₁₂₃ plasmids and the eight pHW plasmids both displayed similar NA activities and HAU titers, indicating that the delay in the rescue kinetics is not amplified when pHW/O₁₂₃ is used as an eight-plasmid system (FIG. 7D). In contrast, only the PR8 virus generated from the pHW/O₁₂₃-N1₉₉ plasmid (PR8N1/99*) produced measurable NA activity and HAU titers by 96 hours post-transfection (FIG. 7D). Upon passaging the cell culture media from the rescues in eggs, all four viruses grew, and no significant differences were observed in the HAU titers of the infected eggs (FIG. 7E) or the protein profiles of the isolated virions (FIG. 7F). These results show that the more stable pHW/O₁₂₃ plasmid can be used in combination with the parental pHW RG system or as a stand-alone eight plasmid system to generate recombinant viruses without any substantial loss in efficiency.

### Example 8: pHW/O₁₂₃ Allows Unstable Influenza Genes to be Propagated in E. Coli

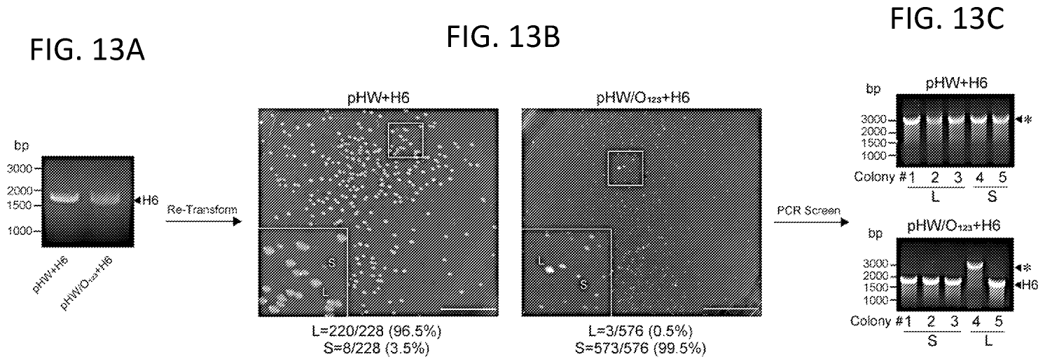

Small scale preparations of pHW-H6 and pHW/O₁₂₃-H6 were sent for commercial DNA production; however substantial changes were found in the pHW-H6 plasmid DNA received from commercial production. Based on this observation, E. coli were re-transformed with the sequence and PCR-verified (FIG. 13A) small scale preparations of pHW-H6 and pHW/O₁₂₃-H6 to determine the stability of the H6 gene segment during propagation of the plasmid DNA in bacteria. Almost all colonies (99.5%) transformed with pHW/O₁₂₃-H6 showed the expected small phenotype (FIG. 13B), whereas almost all colonies (96.5%) transformed with pHW-H6 were large (FIG. 13B). PCR screening of randomly selected small and large E. coli colonies transformed with pHW/O₁₂₃-H6 produced a band at the expected size for full-length H6, whereas all colonies transformed with pHW-H6 yielded a larger than expected band, indicating that the increased stability provided by pHW/O₁₂₃ is equally important for plasmid propagation (FIG. 13C). These data demonstrate that the pHW/O₁₂₃ plasmid can accommodate unstable influenza genes while maintaining the ability to efficiently generate recombinant IAVs.

### Example 9: Expression of Genes Placed Between Two Operators is Inducible in E. Coli

In this example, a commercial pET21 vector that has a T7 promoter followed by a single operator on the 5′ end of the gene for inducible recombinant protein expression in E. coli was used to show incorporation of the 3′ operator (second operator) still supports inducible expression in E. coli. These findings demonstrate that this approach can be used to stabilize genes for cloning DNA and for recombinant protein expression.

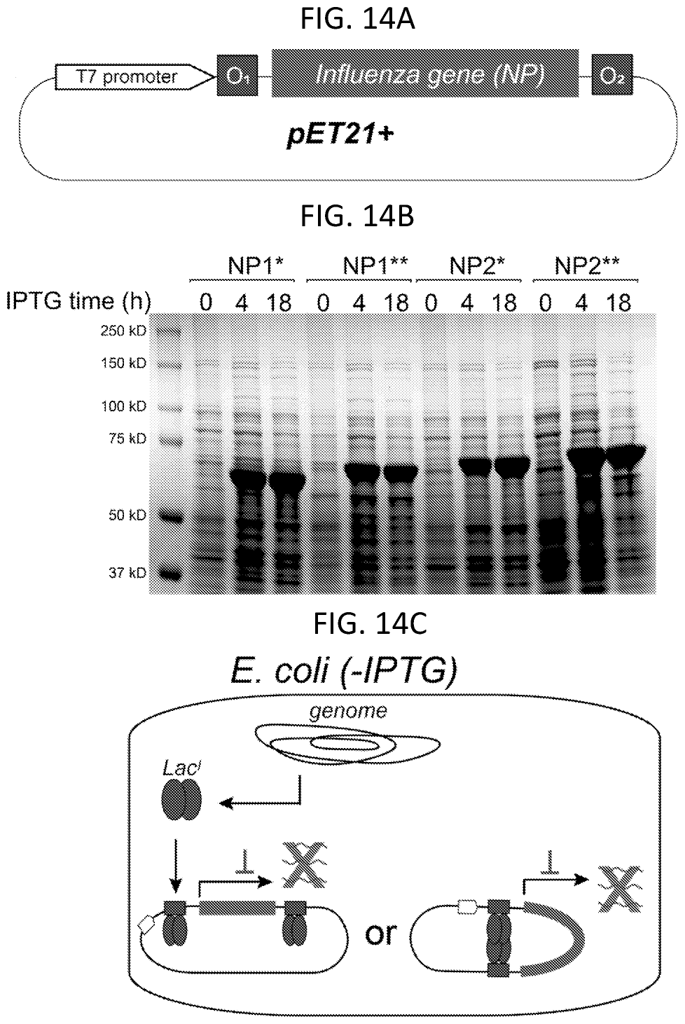

FIG. 14A shows a diagram of the bacterial expression plasmid with the nucleoprotein (NP) influenza gene inserted between two operator sequences (O₁ and O₂). Expression of four NP variants following the addition of 0.4 mM IPTG was shown using a Coomassie stained gel (FIG. 14B). Included were four NP variants with two different N-terminal (*) and C-terminal (**) fusions. Equal volumes of E. coli were sedimented and lysed by sonication, and sample amounts were adjusted for biomass as follows: 15 μl, 10 μl and 4 μl were loaded for each 0-, 4-, and 18-hour sample, respectively. FIG. 14C shows a schematic illustrating two potential mechanisms by which the use of 5′ and 3′ flanking operators can silence gene expression in E. coli through LacI binding, which differs from commercial vectors that only use operators upstream of the 5′ region of the gene. Upon IPTG addition, LacI is released, enabling transcription and translation to occur.

It will be apparent that the precise details of the methods or compositions described may be varied or modified without departing from the spirit of the described aspects of the disclosure. We claim all such modifications and variations that fall within the scope and spirit of the claims below.

1. A recombinant nucleic acid molecule, comprising in the 5′ to 3′ direction:
a first lac operator sequence;
a heterologous DNA sequence, or a multiple cloning site for the insertion of a heterologous DNA sequence; and
a second lac operator sequence.

2. (canceled)

3. The recombinant nucleic acid molecule of claim 1, further comprising a first promoter located 5′ of the first lac operator sequence or located 3′ of the second lac operator sequence.

4. The recombinant nucleic acid molecule of claim 3, comprising a first promoter located 5′ of the first lac operator sequence and a second promoter located 3′ of the second lac operator sequence.

5. The recombinant nucleic acid molecule of claim 3, wherein the first promoter is a bacterial promoter or a mammalian promoter.

6. The recombinant nucleic acid molecule of claim 3, wherein the second promoter is a bacterial promoter or a mammalian promoter.

7. The recombinant nucleic acid molecule of claim 5, wherein:
the bacterial promoter is an E. coli RNA polymerase promoter, T7 promoter or T4 promoter; and/or
the mammalian promoter is an RNA polymerase I promoter, RNA polymerase II promoter or RNA polymerase III promoter.

8. The recombinant nucleic acid molecule of claim 3, further comprising a third lac operator sequence located 5′ of the first promoter or located 3′ of the second promoter.

9. The recombinant nucleic acid molecule of claim 8, comprising in the 5′ to 3′ direction:
the third lac operator sequence, the first promoter, the first lac operator sequence, the heterologous DNA sequence, the second lac operator sequence, the second promoter and a fourth lac operator sequence; or
the third lac operator sequence, the first promoter, the first lac operator sequence, the multiple cloning site for the insertion of the heterologous DNA sequence, the second lac operator sequence, the second promoter and a fourth lac operator sequence.

10. The recombinant nucleic acid molecule of claim 9, wherein the first, second, third and/or fourth lac operator sequence is capable of binding the Escherichia coli Lac repressor protein or a variant thereof having at least 90% sequence identity to SEQ ID NO: 5.

11. The recombinant nucleic acid molecule of claim 10, wherein the Lac repressor protein comprises the amino acid sequence of SEQ ID NO: 5.

12. The recombinant nucleic acid molecule of claim 8, wherein the first lac operator sequence, the second lac operator sequence and the third lac operator sequence are each individually selected from SEQ ID NO: 1, SEQ ID NO: 2, and SEQ ID NO: 3.

13. The recombinant nucleic acid molecule of claim 8, wherein the first lac operator sequence comprises SEQ ID NO: 1, the second lac operator sequence comprises SEQ ID NO: 2 and/or the third lac operator sequence comprises SEQ ID NO: 3.

14. The recombinant nucleic acid molecule of claim 1, further comprising a sequence encoding an Escherichia coli Lac repressor protein or a variant thereof.

15. The recombinant nucleic acid molecule of claim 1, further comprising a terminator sequence located between the first lac operator sequence and the heterologous DNA sequence, or located 3′ of the second lac operator sequence.

16. The recombinant nucleic acid molecule of claim 15, wherein the terminator sequence comprises SEQ ID NO: 4.

17. The recombinant nucleic acid molecule of claim 1, wherein the heterologous DNA sequence encodes a gene or transcript that is toxic to E. coli.

18. The recombinant nucleic acid molecule of claim 1, wherein the heterologous DNA sequence encodes a viral protein.

19. The recombinant nucleic acid molecule of claim 18, wherein the viral protein is an influenza virus protein.

20. The recombinant nucleic acid molecule of claim 19, wherein the influenza virus protein is hemagglutinin or neuraminidase.

21. A plasmid comprising the recombinant nucleic acid molecule of claim 1.

22. The plasmid of claim 21, wherein the plasmid further comprises an origin of replication, a selectable marker gene, a ribosome binding site, a gene termination signal, or any combination thereof.

23. The plasmid of claim 21, wherein the nucleotide sequence of the plasmid comprises or consists of SEQ ID NO: 8, SEQ ID NO: 9, SEQ ID NO: 10, SEQ ID NO: 11, SEQ ID NO: 12 or SEQ ID NO: 13.

24. A plasmid, comprising in the 5′ to 3′ direction:
a lac operator sequence comprising SEQ ID NO: 3;
a promoter;
a lac operator sequence comprising SEQ ID NO: 1;
an influenza virus hemagglutinin or neuraminidase gene; and
a lac operator sequence comprising SEQ ID NO: 2.

25. A plasmid, comprising in the 5′ to 3′ direction:
a lac operator sequence comprising SEQ ID NO: 3;
a promoter;
a lac operator sequence comprising SEQ ID NO: 1;
a terminator sequence comprising SEQ ID NO: 4;
an influenza virus hemagglutinin or neuraminidase gene; and
a lac operator sequence comprising SEQ ID NO: 2.

26. A method of propagating a plasmid in E. coli, wherein the plasmid comprises a heterologous DNA sequence that is toxic to E. coli, comprising:
transforming E. coli with the plasmid of claim 21 under conditions sufficient to allow replication of the plasmid, thereby propagating the plasmid in E. coli.

27. The method of claim 26, wherein the heterologous DNA sequence toxic to E. coli is an influenza virus gene.

28. The method of claim 27, wherein the influenza virus gene is the hemagglutinin gene.

29. The method of claim 27, wherein the influenza virus gene is the neuraminidase gene.

30. A kit, comprising:
the recombinant nucleic acid molecule of claim 1; and
one or more restriction endonucleases, buffer, culture media, one or more ligases, primers, reverse transcriptase, deoxyribonucleotide triphosphates (dNTPs), one or more antibiotics, cells, an inducer, or a combination thereof.

31. The kit of claim 30, wherein the cells are prokaryotic cells.

32. The kit of claim 31, wherein the prokaryotic cells are Escherichia coli cells

33. The kit of claim 30, wherein the cells are eukaryotic cells.

34. The kit of claim 30, wherein the inducer is isopropyl β-D-1-thiogalactopyranoside (IPTG).

35. An isolated cell, comprising the recombinant nucleic acid molecule of claim 1, wherein the recombinant nucleic acid molecule is in a complex with an Escherichia coli Lac repressor protein or a variant thereof having at least 90% sequence identity to SEQ ID NO: 5.

36. The isolated cell of claim 35, wherein the Lac repressor protein comprises the amino acid sequence of SEQ ID NO: 5.
